(19) United States
(12) Patent Application Publication
(10) Pub. No.: US 2024/0391972 A1
(43) Pub. Date: Nov. 28, 2024

(54) IL-12 AND IL-15 POLYPEPTIDES

(71) Applicant: SAGITTARIUS BIO, INC, Thousand Oaks, CA (US)

(72) Inventors: Kayvan NIAZI, Thousand Oaks, CA (US); Shahrooz RABIZADEH, Thousand Oaks, CA (US)

(21) Appl. No.: 18/694,880

(22) PCT Filed: Sep. 23, 2022
(86) PCT No.: PCT/US2022/076921

§ 371 Date: Mar. 22, 2024

Related U.S. Application Data
(60) Provisional application No. 63/362,312, filed on Mar. 31, 2022.
(60) Provisional application No. 63/261,560, filed on Sep. 23, 2021.

(51) Int. Cl. C07K 14/54; A61K 38/00; A61K 39/00; C07K 14/715

(52) U.S. Cl. CPC C07K 14/5434; A61K 39/4611; A61K 39/4635; C07K 14/5443; C07K 14/7155; A61K 38/00; C07K 2319/00

(57) ABSTRACT

The present application provides for polypeptides comprising an IL-12 polypeptide, and/or a IL-15 polypeptide, and/or a IL-15 receptor polypeptide, compositions comprising the same, and methods of using the same.

### PRIORITY

The present application claims priority to U.S. Provisional Application 63/261,560, filed on Sep. 23, 2021, and U.S. Provisional Application 63/362,312, filed Mar. 31, 2022, each of which are hereby incorporated by reference in their entireties.

### SEQUENCE LISTING

The present application contains a Sequence Listing which has been submitted electronically is XML format and is hereby incorporated by reference in its entirety. The Sequence Listing, created on Sep. 19, 2022, is called “260034.000302 sequence listing XML” and is 87,413 bytes in size.

### FIELD

Embodiments provided herein relate to polypeptides comprising a IL-15 polypeptide and a IL-12 polypeptide, compositions comprising the same, and methods of using the same.

### BACKGROUND

IL-12 and IL-15 are cytokines that modulate and activate an immune response to treat conditions, such as cancer and infections. However, there is a need for improved polypeptides that can be used to stimulate the immune system in a localized or systemic manner. The present embodiments fulfill these needs as well as others.

### SUMMARY

In some embodiments, polypeptides are provided that comprises polypeptides comprising one or more of a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Ra) polypeptide.

In some embodiments, polypeptide are provided that comprise the formula of: X₁-L₁-X₂-L₂-X₃-L₃-X₄, wherein: X₁ is a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Ra) polypeptide; X₂ is a IL-15 polypeptide, IL-12 p40 polypeptide, IL-12 p35 polypeptide, or IL-15Ra polypeptide; X₃ is a IL-15 polypeptide, IL-12 p40 polypeptide, IL-12 p35 polypeptide, or IL-15Ra polypeptide; X₄ is a IL-15 polypeptide, IL-12 p40 polypeptide, IL-12 p35 polypeptide, or IL-15Ra polypeptide; L₁, L₂, and L₃ are each, independently, a polypeptide linker that comprise the same or different polypeptide sequences, provided each of X₁, X₂, X₃, and X₄ are different.

In some embodiments, polypeptides are provided that comprise the formula of: X₁-L₁-X₂-L₂-X₃-L₃-X₄-L₄-X₅, wherein: X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Ra) polypeptide, a first fragment of IL-15Ra polypeptide, or a second fragment of IL-15Ra polypeptide; X₂ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide; X₃ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide; X₄ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide; X₅ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide; L₁, L₂, L₃, and L₄ are each, independently, a polypeptide linker that comprise the same or different polypeptide sequences, provided each of X₁, X₂, X₃, X₄, and X₅ are different.

In some embodiments, polypeptides are provided that comprise the formula of: X₁-L₁-X₂, wherein: X₁ is a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Ra) polypeptide; X₂ is a IL-15 polypeptide, IL-12 p40 polypeptide, IL-12 p35 polypeptide, or IL-15Ra polypeptide; L₁ is a polypeptide linker, provided X₁ and X₂ are different.

In some embodiments, polypeptides are provided that comprise the formula of: X₁-L₁-X₂-L₂-X₃, wherein: X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Ra) polypeptide, a first fragment of IL-15Ra polypeptide, or a second fragment of IL-15Ra polypeptide; X₂ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide; X₃ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide; L₁ and L₂ are each, independently, a polypeptide linker that comprise the same or different polypeptide sequences, provided each of X₁, X₂, and X₃ are different.

In some embodiments, pharmaceutical compositions comprising any of the polypeptides described herein are provided. In some embodiments, nucleic acid molecules encoding any of the polypeptides described herein are provided. In some embodiments, vectors comprising any of the nucleic acid molecules described herein are provided. In some embodiments, plasmids comprising any of the nucleic acid molecules described herein are provided. In some embodiments, recombinant viruses comprising any of the nucleic acid molecules described herein are provided. In some embodiments, cells comprising any of the polypeptides described herein are provided.

In some embodiments, a method for producing any cell described herein is provided, the method comprising contacting the cell with any pharmaceutical composition, vector, plasmid, or virus described herein. In some embodiments, a method of producing a cell comprising any polypeptide described herein is provided, the method comprising administering to a subject any vector, plasmid, or virus described herein, wherein the vector, plasmid, or virus transduces or transfects a cell in vivo. In some embodiments, a method for modifying an immune response in a patient is provided, the method comprising administering to the patient any pharmaceutical composition, vector, plasmid, or virus described herein. In some embodiments, a method of treating a cancer is a subject is provided, the method comprising administering to the subject any pharmaceutical composition, vector, plasmid, or virus described herein. In some embodiments, a method of treating a disease or disorder, such as a viral infection, bacterial infection or a fungal infection, such as those provided herein, is provided, the method comprising administering to the subject any pharmaceutical composition, vector, plasmid, or virus described herein.

### DETAILED DESCRIPTION

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “individual” or “subject,” or “patient” used interchangeably, means any animal, including mammals, such as mice, rats, other rodents, rabbits, dogs, cats, swine, cattle, sheep, horses, or primates, such as humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

As used herein, the term “contacting” means bringing together of two elements in an in vitro system or an in vivo system. For example, “contacting” a vector with a cell or with an individual or patient or cell includes the administration of the vector to an individual or patient, such as a human, as well as, for example, introducing a compound into a sample containing a cellular or purified preparation containing the cell.

As used herein, the term “fused” or “linked” when used in reference to a protein having different domains or heterologous sequences means that the protein domains are part of the same peptide chain that are connected to one another with either peptide bonds or other covalent bonding. The domains or fragments can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as the glycine/serine sequences described herein link the two domains together. These are also provided for herein with non-limiting examples.

The terms “substituting,” “substituted,” “mutating,” or “mutated” as used herein refer to altering, deleting, or inserting one or more amino acids or nucleotides in a polypeptide or polynucleotide sequence to generate a variant of that sequence.

The term “variant” as used herein refers to a polypeptide or polynucleotide that differs from a reference polypeptide or a reference polynucleotide by one or more modifications, including substitutions, insertions, or deletions.

The term “vector” means a composition of matter that can be used to deliver a cargo to a target, such as a cell, tissue, organ, and the like. In some embodiments, the vector is capable of being duplicated, which can be referred to as a “replicating vector.” In some embodiments, the vector is a non-replicating vector. In some embodiments, the vector is produced in a packaging cell line. In some embodiments, the vector is a viral vector, such as, but not limited to an adenoviral vector. Adenoviral vectors (“adenoviruses”) can be produced to be non-replicating by deleting genes necessary from replication from the adenoviral genome. For example, the E1, E2, E3, or E4 genes can be deleted, either singularly or in combination with one another. To produce the adenoviral particle, the polypeptides produced by these genes are provided by a packaging cell line. Methods of producing adenoviral particles are well known in the art. In some embodiments, the vector can contain elements, such as origins of replication, polyadenylation signal or selection markers that function to facilitate the duplication or maintenance of these polynucleotides in a biological system.

The term “expression vector” means a vector that can be utilized in a biological system, such as, but not limited to, a cell, tissue, or organ, or in a reconstituted biological system to direct the translation of a polypeptide encoded by a polynucleotides sequence present in the expression vector.

The terms “polynucleotide” or “nucleic acid molecule” means a molecule comprising a chain of nucleotides covalently linked by a sugar-phosphate backbone or other equivalent covalent chemistry. Double and single-stranded DNAs and RNAs are typical example of polynucleotides.

The term “polypeptide” or “protein” means a molecule that comprises at least two amino acid resides linked by a peptide bond to form a polypeptide. In some embodiments, the term “peptide” can also be used.

### Compositions

Described herein are polypeptides comprising an IL-12 polypeptide and a IL-15 polypeptide. In some embodiments, the polypeptide comprises a IL-12 p40 polypeptide, an IL-p35 polypeptide, an IL-15 polypeptide, a IL-15Rα polypeptide, and/or a first or second fragment of a IL-15Rα polypeptide. In some embodiments, the IL-12 p40 polypeptide is from a human. In some embodiments, the IL-12 p40 polypeptide is from a mouse. In some embodiments, the IL-12 p35 polypeptide is from a human. In some embodiments, the IL-12 p35 polypeptide is from a mouse. In some embodiments, the polypeptide can be used to stimulate an immune response. In some embodiments, the polypeptides can be used to activate by NK cells or CD8+ T cells. In some embodiments, the polypeptides can be used to treat cancer, such as those provided for herein, viral infections, bacterial infections, such as, but not limited to tuberculosis, listeriosis, and the like, and fungal infections.

In some embodiments, the IL-12 polypeptide refers to a polypeptide that comprises a functional p40 polypeptide and a p35 polypeptide. These polypeptides can be expressed together to form the IL-12 polypeptide or can be linked through the use of a linker to link the p35 and p40 polypeptides together. In some embodiments, the linker is a polypeptide linker, such as those provided herein.

In some embodiments, the IL-12 p40 polypeptide comprises an amino acid sequence of: MCHQQLVISWFSLVFLASPLVAIWELKKDVYVVELDWYPDAPGEMVVLTCDTPEEDGI TWTLDQSSEVLGSGKTLTIQVKEFGDAGQYTCHKGGEVLSHSLLLLHKKEDGIWSTDIL KDQKEPKNKTFLRCEAKNYSGRFTCWWLTTISTDLTFSVKSSRGSSDPQGVTCGAATLS AERVRGDNKEYEYSVECQEDSACPAAEESLPIEVMVDAVHKLKYENYTSSFFIRDIIKPD PPKNLQLKPLKNSRQVEVSWEYPDTWSTPHSYFSLTFCVQVQGKSKREKKDRVFTDKT SATVICRKNASISVRAQDRYYSSSWSEWASVPCS (SEQ ID NO: 1), or an active fragment thereof. The IL-12 p40 polypeptide can be processed to produce a mature polypeptide, which can be referred to as the active portion of the polypeptide. In some embodiments, the IL-12 p40 polypeptide, or active fragment thereof, comprises an amino acid sequence of: IWELKKDVYVVELDWYPDAPGEMVVLTCDTPEEDGITWTLDQSSEVLGSGKTLTIQVK EFGDAGQYTCHKGGEVLSHSLLLLHKKEDGIWSTDILKDQKEPKNKTFLRCEAKNYSGR FTCWWLTTISTDLTFSVKSSRGSSDPQGVTCGAATLSAERVRGDNKEYEYSVECQEDSA CPAAEESLPIEVMVDAVHKLKYENYTSSFFIRDIIKPDPPKNLQLKPLKNSRQVEVSWEY PDTWSTPHSYFSLTFCVQVQGKSKREKKDRVFTDKTSATVICRKNASISVRAQDRYYSS SWSEWASVPCS (SEQ ID NO: 2). In some embodiments, 1, 2, 3, 4, or 5 amino acid residues are deleted from the N-terminus of SEQ ID NO: 2 to be the IL-12 p40 polypeptide. In some embodiments, 1, 2, 3, 4, or 5 amino acid residues are deleted from the C-terminus of SEQ ID NO: 2 to be the IL-12 p40 polypeptide. In some embodiments, 1, 2, 3, 4, or 5 amino acid residues are deleted from the N-terminus and/or the C-terminus of SEQ ID NO: 2 to be the IL-12 p40 polypeptide. In some embodiments, SEQ ID NOs: 1 and 2 are or are derived from human IL-12 p40.

In some embodiments, the IL-12 p40 polypeptide comprises an amino acid sequence of: MCPQKLTISWFAIVLLVSPLMAMWELEKDVYVVEVDWTPDAPGETVNLTCDTPEEDDI TWTSDQRHGVIGSGKTLTITVKEFLDAGQYTCHKGGETLSHSHLLLHKKENGIWSTEILK NFKNKTFLKCEAPNYSGRFTCSWLVQRNMDLKFNIKSSSSSPDSRAVTCGMASLSAEKV TLDQRDYEKYSVSCQEDVTCPTAEETLPIELALEARQQNKYENYSTSFFIRDIIKPDPPKN LQMKPLKNSQVEVSWEYPDSWSTPHSYFSLKFFVRIQRKKEKMKETEEGCNQKGAFLV EKTSTEVQCKGGNVCVQAQDRYYNSSCSKWACVPCRVRS (SEQ ID NO: 3), or an active fragment thereof. The IL-12 p40 polypeptide can be processed to produce a mature polypeptide, which can be referred to as the active portion of the polypeptide. In some embodiments, the IL-12 p40 polypeptide, or active fragment thereof, comprises an amino acid sequence of: MWELEKDVYVVEVDWTPDAPGETVNLTCDTPEEDDITWTSDQRHGVIGSGKTLTITVK EFLDAGQYTCHKGGETLSHSHLLLHKKENGIWSTEILKNFKNKTFLKCEAPNYSGRFTCS WLVQRNMDLKFNIKSSSSSPDSRAVTCGMASLSAEKVTLDQRDYEKYSVSCQEDVTCP TAEETLPIELALEARQQNKYENYSTSFFIRDIIKPDPPKNLQMKPLKNSQVEVSWEYPDS WSTPHSYFSLKFFVRIQRKKEKMKETEEGCNQKGAFLVEKTSTEVQCKGGNVCVQAQD RYYNSSCSKWACVPCRVRS (SEQ ID NO: 4). In some embodiments, 1, 2, 3, 4, or 5 amino acid residues are deleted from the N-terminus of SEQ ID NO: 4 to be the IL-12 p40 polypeptide. In some embodiments, 1, 2, 3, 4, or 5 amino acid residues are deleted from the C-terminus of SEQ ID NO: 4 to be the IL-12 p40 polypeptide. In some embodiments, 1, 2, 3, 4, or 5 amino acid residues are deleted from the N-terminus and/or the C-terminus of SEQ ID NO: 4 to be the IL-12 p40 polypeptide. In some embodiments, SEQ ID NOs: 3 and 4 are or are derived from mouse IL-12 p40.

In some embodiments, the IL-12 p35 polypeptide comprises an amino acid sequence of: MCPARSLLLVATLVLLDHLSLARNLPVATPDPGMFPCLHHSQNLLRAVSNMLQKARQT LEFYPCTSEEIDHEDITKDKTSTVEACLPLELTKNESCLNSRETSFITNGSCLASRKTSFM MALCLSSIYEDLKMYQVEFKTMNAKLLMDPKRQIFLDQNMLAVIDELMQALNFNSETV PQKSSLEEPDFYKTKIKLCILLHAFRIRAVTIDRVMSYLNAS (SEQ ID NO: 5), or an active fragment thereof. The IL-12 p35 polypeptide can be processed to produce a mature polypeptide, which can be referred to as the active portion of the polypeptide. In some embodiments, the IL-12 p35 polypeptide, or active fragment thereof, comprises an amino acid sequence of: RNLPVATPDPGMFPCLHHSQNLLRAVSNMLQKARQTLEFYPCTSEEIDHEDITKDKTST VEACLPLELTKNESCLNSRETSFITNGSCLASRKTSFMMALCLSSIYEDLKMYQVEFKTM NAKLLMDPKRQIFLDQNMLAVIDELMQALNFNSETVPQKSSLEEPDFYKTKIKLCILLHA FRIRAVTIDRVMSYLNAS (SEQ ID NO: 6). In some embodiments, 1, 2, 3, 4, or 5 amino acid residues are deleted from the N-terminus of SEQ ID NO: 6 to be the IL-12 p35 polypeptide. In some embodiments, 1, 2, 3, 4, or 5 amino acid residues are deleted from the C-terminus of SEQ ID NO: 6 to be the IL-12 p35 polypeptide. In some embodiments, 1, 2, 3, 4, or 5 amino acid residues are deleted from the N-terminus and/or the C-terminus of SEQ ID NO: 6 to be the IL-12 p35 polypeptide. In some embodiments, SEQ ID NOs: 5 and 6 are or are derived from human IL-12 p35.

In some embodiments, the IL-12 p35 polypeptide comprises an amino acid sequence of: MCQSRYLLFLATLALLNHLSLARVIPVSGPARCLSQSRNLLKTTDDMVKTAREKLKHYS CTAEDIDHEDITRDQTSTLKTCLPLELHKNESCLATRETSSTTRGSCLPPQKTSLMMTLCL GSIYEDLKMYQTEFQAINAALQNHNHQQIILDKGMLVAIDELMQSLNHNGETLRQKPPV GEADPYRVKMKLCILLHAFSTRVVTINRVMGYLSSA (SEQ ID NO: 7), or an active fragment thereof. The IL-12 p35 polypeptide can be processed to produce a mature polypeptide, which can be referred to as the active portion of the polypeptide. In some embodiments, the IL-12 p35 polypeptide, or active fragment thereof, comprises an amino acid sequence of: RVIPVSGPARCLSQSRNLLKTTDDMVKTAREKLKHYSCTAEDIDHEDITRDQTSTLKTCL PLELHKNESCLATRETSSTTRGSCLPPQKTSLMMTLCLGSIYEDLKMYQTEFQAINAALQ NHNHQQIILDKGMLVAIDELMQSLNHNGETLRQKPPVGEADPYRVKMKLCILLHAFSTR VVTINRVMGYLSSA (SEQ ID NO: 8). In some embodiments, 1, 2, 3, 4, or 5 amino acid residues are deleted from the N-terminus of SEQ ID NO: 8 to be the IL-12 p35 polypeptide. In some embodiments, 1, 2, 3, 4, or 5 amino acid residues are deleted from the C-terminus of SEQ ID NO: 8 to be the IL-12 p35 polypeptide. In some embodiments, 1, 2, 3, 4, or 5 amino acid residues are deleted from the N-terminus and/or the C-terminus of SEQ ID NO: 8 to be the IL-12 p35 polypeptide. In some embodiments, SEQ ID NOs: 7 and 8 are or are derived from mouse IL-12 p35.

In some embodiments, the IL-15a polypeptide comprises an amino acid sequence of: MRISKPHLRSISIQCYLCLLLNSHFLTEAGIHVFILGCFSAGLPKTEANWVNVISDLKKIED LIQSMHIDATLYTESDVHPSCKVTAMKCFLLELQVISLESGDASIHDTVENLIILANNSLSS NGNVTESGCKECEELEEKNIKEFLQSFVHIVQMFINTS (SEQ ID NO: 9), or an active fragment thereof. The IL-15 polypeptide can be processed to produce a mature polypeptide, which can be referred to as the active portion of the polypeptide. In some embodiments, the IL-15 polypeptide, or active fragment thereof, comprises an amino acid sequence of: GIHVFILGCFSAGLPKTEANWVNVISDLKKIEDLIQSMHIDATLYTESDVHPSCKVTAMK CFLLELQVISLESGDASIHDTVENLIILANNSLSSNGNVTESGCKECEELEEKNIKEFLQSF VHIVQMFINTS (SEQ ID NO: 10). In some embodiments, 1, 2, 3, 4, or 5 amino acid residues are deleted from the N-terminus of SEQ ID NO: 10 to be the IL-15 polypeptide. In some embodiments, 1, 2, 3, 4, or 5 amino acid residues are deleted from the C-terminus of SEQ ID NO: 10 to be the IL-15 polypeptide. In some embodiments, 1, 2, 3, 4, or 5 amino acid residues are deleted from the N-terminus and/or the C-terminus of SEQ ID NO: 6 to be the IL-15 polypeptide.

In some embodiments, the IL-15Rα polypeptide comprises an amino acid sequence of: LQITCPPPMSVEHADIWVKSYSLYSRERYICNSGFKRKAGTSSLTECVLNKATNVAHWT TPSLKCIRDPALVHQRPAPPSTVTTAGVTPQPESLSPSGKEPAASSPSSNNTAATTAAIVP GSQLMPSKSPSTGTTEISSHESSHGTPSQTTAKNWELTASASHQPPGVYPQGHSDTTVAIS TSTVLLCGLSAVSLLACYLKSRQTPPLASVEMEAMEALPVTWGTSSRDEDLENCSHHL (SEQ ID NO: 11), or an active fragment thereof. In some embodiments, the IL-15Rα polypeptide, or active fragment thereof, comprises an amino acid sequence of: ITCPPPMSVEHADIWVKSYSLYSRERYICNSGFKRKAGTSSLTECVLNKATNVAHWTTPS LKCIRDPALVHQRPAPPSTVTTAGVTPQPESLSPSGKEPAASSPSSNNTAATTAAIVPGSQ LMPSKSPSTGTTEISSHESSHGTPSQTTAKNWELTASASHQPPGVYPQGHSDTTVAISTST VLLCGLSAVSLLACYLKSRQTPPLASVEMEAMEALPVTWGTSSRDEDLENCSHHL (SEQ ID NO: 12). In some embodiments, the first fragment of the IL-15Rα polypeptide comprises an amino acid sequence of: ITCPPPMSVEHADIWVKSYSLYSRERYICNSGFKRKAGTSSLTECVLNKATNVAHWTTPS LKCIRDPALVHQR (SEQ ID NO:13). In some embodiments, the second fragment of the IL-15Rα polypeptide comprises an amino acid sequence of: PAPPSTVTTAGVTPQPESLSPSGKEPAASSPSSNNTAATTAAIVPGSQLMPSKSPSTGTTEI SSHESSHGTPSQTTAKNWELTASASHQPPGVYPQGHSDTTVAISTSTVLLCGLSAVSLLA CYLKSRQTPPLASVEMEAMEALPVTWGTSSRDEDLENCSHHL (SEQ ID NO: 14). In some embodiments, 1, 2, 3, 4, or 5 amino acid residues are deleted from the N-terminus of SEQ ID NO: 11, SEQ ID NO: 12, SEQ ID NO: 13, or SEQ ID NO: 14 to be the IL-15Rα polypeptide. In some embodiments, 1, 2, 3, 4, or 5 amino acid residues are deleted from the C-terminus of SEQ ID NO: 11, SEQ ID NO: 12, SEQ ID NO: 13, or SEQ ID NO: 14 to be the IL-15Ra. In some embodiments, 1, 2, 3, 4, or 5 amino acid residues are deleted from the N-terminus and/or the C-terminus of SEQ ID NO: 11, SEQ ID NO: 12, SEQ ID NO: 13, or SEQ ID NO: 14 to be the IL-15Rα.

There are other isoforms of IL-15Rα that can be modified to produce an active polypeptide. The sequences of such isoforms can be found, for example at GenBank Accession Nos.: NP_002180.1, AEP26933.1, AAP69528.1, AAH74726.1, NP_001243694.1, XP_016871684.1, XP_011517767.1, NP_001230468.1, and AAI07778.1, each of which is hereby incorporated by reference in its entirety.

Although the sequences provided herein are human and mouse sequences, other orthologs can also be used because certain orthologs of the same proteins have the same or similar activity in a different species. For example, mouse IL-12 is active on human cells, and, therefore, the p40 and/or p35 human subunits of IL-12 can be replaced with the mouse orthologs provided herein. Similarly, the IL-15 human form can be used in other animals because of the high degree of similarity (identity).

Accordingly, in some embodiments, polypeptides having the formula of X₁-L₁-X₂-L₂-X₃-L₃-X₄, are provided, wherein:
X1 is an interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Rα) polypeptide;
X2 is a IL-15 polypeptide, IL-12 p40 polypeptide, IL-12 p35 polypeptide, or IL-15Rα polypeptide;
X3 is a IL-15 polypeptide, IL-12 p40 polypeptide, IL-12 p35 polypeptide, or IL-15Rα polypeptide;
X4 is a IL-15 polypeptide, IL-12 p40 polypeptide, IL-12 p35 polypeptide, or IL-15Rα polypeptide;
L1, L2 and L3 are each, independent, a polypeptide linker that comprise the same or different polypeptide sequences,
provided each of X1, X2, X3, and X4 are different.

In some embodiments, X1 is a IL-12 p40 polypeptide; X2 is a IL-12 p35 polypeptide; X3 is an IL-15 polypeptide; and X4 is a IL-15Rα polypeptide. In some embodiments, X1 is a IL-12 p35 polypeptide; X2 is a IL-12 p40 polypeptide; X3 is an IL-15 polypeptide; and X4 is a IL-15Rα polypeptide. In some embodiments, X1 is an IL-15 polypeptide; X2 is a IL-12 p40 polypeptide; X3 is a IL-12 p35 polypeptide; and X4 is a IL-15Rα polypeptide. In some embodiments, X1 is an IL-15 polypeptide; X2 is a IL-12 p35 polypeptide; X3 is a IL-12 p40 polypeptide; and X4 is a IL-15Rα polypeptide. In some embodiments, X1 is a IL-12 p35 polypeptide; X2 is an IL-15 polypeptide; X3 is a IL-12 p40 polypeptide; and X4 is a IL-15Rα polypeptide. In some embodiments, X1 is a IL-12 p40 polypeptide; X2 is an IL-15 polypeptide; X3 is a IL-12 p35 polypeptide; and X4 is a IL-15Rα polypeptide.

In some embodiments, polypeptides comprising the formula of: X₁-L₁-X₂-L₂-X₃-L₃-X₄-L₄-X₅ are provided, wherein:
X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Rα) polypeptide, a first fragment of IL-15Rα polypeptide, or a second fragment of IL-15Rα polypeptide;
X₂ is a IL-15 polypeptide, IL-12 p40 polypeptide, IL-12 p35 polypeptide, IL-15Rα polypeptide, a first fragment of IL-15Rα polypeptide, or a second fragment of IL-15Rα polypeptide;
X₃ is a IL-15 polypeptide, IL-12 p40 polypeptide, IL-12 p35 polypeptide, IL-15Rα polypeptide, a first fragment of IL-15Rα polypeptide, or a second fragment of IL-15Rα polypeptide;
X₄ is a IL-15 polypeptide, IL-12 p40 polypeptide, IL-12 p35 polypeptide, IL-15Rα polypeptide, a first fragment of IL-15Rα polypeptide, or a second fragment of IL-15Rα polypeptide;
X₅ is a IL-15 polypeptide, IL-12 p40 polypeptide, IL-12 p35 polypeptide, IL-15Rα polypeptide, a first fragment of IL-15Rα polypeptide, or a second fragment of IL-15Rα polypeptide;
L₁, L₂, L₃, and L₄ are each, independently, a polypeptide linker that comprise the same or different polypeptide sequences,
provided each of X₁, X₂, X₃, X₄, and X₅ are different.

In some embodiments, X₁ is a IL-15 polypeptide; X₂ is a first fragment of IL-15Rα polypeptide; X₃ is a IL-12 p40 polypeptide; X₄ is a IL-12 p35 polypeptide; and X₅ is a second fragment of IL-15Rα polypeptide. In some embodiments, X₁ is a IL-15 polypeptide; X₂ is a first fragment of IL-15Rα polypeptide; X₃ is a IL-12 p35 polypeptide; X₄ is a IL-12 p40 polypeptide; and X₅ is a second fragment of IL-15Rα polypeptide. In some embodiments, X₁ is a IL-15 polypeptide; X₂ is a IL-12 p40 polypeptide; X₃ is a first fragment of IL-15Rα polypeptide; X₄ is a IL-12 p35 polypeptide; and X₅ is a second fragment of IL-15Rα polypeptide. In some embodiments, X₁ is a IL-15 polypeptide; X₂ is a IL-12 p40 polypeptide; X₃ is a IL-12 p35 polypeptide; X₄ is a first fragment of IL-15Rα polypeptide; and X₅ is a second fragment of IL-15Rα polypeptide. In some embodiments, X₁ is a IL-15 polypeptide; X₂ is a IL-12 p35 polypeptide; X₃ is a IL-12 p40 polypeptide; X₄ is a first fragment of IL-15Rα polypeptide; and X₅ is a second fragment of IL-15Rα polypeptide. In some embodiments, X₁ is a IL-15 polypeptide; X₂ is a IL-12 p35 polypeptide; X₃ is a first fragment of IL-15Rα polypeptide; X₄ is a IL-12 p40 polypeptide; and X₅ is a second fragment of IL-15Rα polypeptide. In some embodiments, X₁ is a first fragment of IL-15Rα polypeptide; X₂ is a IL-15 polypeptide; X₃ is a IL-12 p40 polypeptide; X₄ is a IL-12 p35 polypeptide; and X₅ is a second fragment of IL-15Rα polypeptide. In some embodiments, X₁ is a first fragment of IL-15Rα polypeptide; X₂ is a IL-15 polypeptide; X₃ is a IL-12 p35 polypeptide; X₄ is a IL-12 p40 polypeptide; and X₅ is a second fragment of IL-15Rα polypeptide. In some embodiments, X₁ is a first fragment of IL-15Rα polypeptide; X₂ is a IL-12 p40 polypeptide; X₃ is a IL-15 polypeptide; X₄ is a IL-12 p35 polypeptide; and X₅ is a second fragment of IL-15Rα polypeptide. In some embodiments, X₁ is a first fragment of IL-15Rα polypeptide; X₂ is a IL-12 p40 polypeptide; X₃ is a IL-12 p35 polypeptide; X₄ is a IL-15; and X₅ is a second fragment of IL-15Rα polypeptide. In some embodiments, X₁ is a first fragment of IL-15Rα polypeptide; X₂ is a IL-12 p35 polypeptide; X₃ is a IL-12 p40 polypeptide; X₄ is a IL-15; and X₅ is a second fragment of IL-15Rα polypeptide. In some embodiments, X₁ is a first fragment of IL-15Rα polypeptide; X₂ is a IL-12 p35 polypeptide; X₃ is a IL-15; X₄ is a IL-12 p40 polypeptide; and X₅ is a second fragment of IL-15Rα polypeptide. In some embodiments, X₁ is a IL-12 p40 polypeptide; X₂ is a IL-15 polypeptide; X₃ is a first fragment of IL-15Rα polypeptide; X₄ is a IL-12 p35 polypeptide; and X₅ is a second fragment of IL-15Rα polypeptide. In some embodiments, X₁ is a IL-12 p40 polypeptide; X₂ is a IL-15 polypeptide; X₃ is a IL-12 p35 polypeptide; X₄ is a first fragment of IL-15Rα polypeptide; and X₅ is a second fragment of IL-15Rα polypeptide. In some embodiments, X₁ is a IL-12 p40 polypeptide; X₂ is a first fragment of IL-15Rα polypeptide; X₃ is a IL-15 polypeptide; X₄ is a IL-12 p35 polypeptide; and X₅ is a second fragment of IL-15Rα polypeptide. In some embodiments, X₁ is a IL-12 p40 polypeptide; X₂ is a first fragment of IL-15Rα polypeptide; X₃ is a IL-12 p35 polypeptide; X₄ is a IL-15; and X₅ is a second fragment of IL-15Rα polypeptide. In some embodiments, X₁ is a IL-12 p40 polypeptide; X₂ is a IL-12 p35 polypeptide; X₃ is a first fragment of IL-15Rα polypeptide; X₄ is a IL-15; and X₅ is a second fragment of IL-15Rα polypeptide. In some embodiments, X₁ is a IL-12 p40 polypeptide; X₂ is a IL-12 p35 polypeptide; X₃ is a IL-15; X₄ is a first fragment of IL-15Rα polypeptide; and X₅ is a second fragment of IL-15Rα polypeptide. In some embodiments, X₁ is a IL-12 p35 polypeptide; X₂ is a IL-15 polypeptide; X₃ is a first fragment of IL-15Rα polypeptide; X₄ is a IL-12 p40 polypeptide; and X₅ is a second fragment of IL-15Rα polypeptide. In some embodiments, X₁ is a IL-12 p35 polypeptide; X₂ is a IL-15 polypeptide; X₃ is a IL-12 p40 polypeptide; X₄ is a first fragment of IL-15Rα polypeptide; and X₅ is a second fragment of IL-15Rα polypeptide. In some embodiments, X₁ is a IL-12 p35 polypeptide; X₂ is a first fragment of IL-15Rα polypeptide; X₃ is a IL-15 polypeptide; X₄ is a IL-12 p40 polypeptide; and X₅ is a second fragment of IL-15Rα polypeptide. In some embodiments, X₁ is a IL-12 p35 polypeptide; X₂ is a first fragment of IL-15Rα polypeptide; X₃ is a IL-12 p40 polypeptide; X₄ is a IL-15; and X₅ is a second fragment of IL-15Rα polypeptide. In some embodiments, X₁ is a IL-12 p35 polypeptide; X₂ is a IL-12 p40 polypeptide; X₃ is a first fragment of IL-15Rα polypeptide; X₄ is a IL-15; and X₅ is a second fragment of IL-15Rα polypeptide. In some embodiments, X₁ is a IL-12 p35 polypeptide; X₂ is a IL-12 p40 polypeptide; X₃ is a IL-15; X₄ is a first fragment of IL-15Rα polypeptide; and X₅ is a second fragment of IL-15Rα polypeptide.

In some embodiments, polypeptides comprising the formula of: X₁-L₁-X₂ are provided, wherein:
X₁ is a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Rα) polypeptide;
X₂ is a IL-15 polypeptide, IL-12 p40 polypeptide, IL-12 p35 polypeptide, or IL-15Rα polypeptide;
L₁ is a polypeptide linker,
provided X₁ and X₂ are different.

In some embodiments, X₁ is a IL-15 polypeptide; and X₂ is a IL-15Rα polypeptide.

In some embodiments, polypeptides comprising the formula of: X₁-L₁-X₂-L₂-X₃ are provided, wherein:
X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Rα) polypeptide, a first fragment of IL-15Rα polypeptide, or a second fragment of IL-15Rα polypeptide;
X₂ is a IL-15 polypeptide, IL-12 p40 polypeptide, IL-12 p35 polypeptide, IL-15Rα polypeptide, a first fragment of IL-15Rα polypeptide, or a second fragment of IL-15Rα polypeptide;
X₃ is a IL-15 polypeptide, IL-12 p40 polypeptide, IL-12 p35 polypeptide, IL-15Rα polypeptide, a first fragment of IL-15Rα polypeptide, or a second fragment of IL-15Rα polypeptide;
L₁ and L₂ are each, independently, a polypeptide linker that comprise the same or different polypeptide sequences,
provided each of X₁, X₂, and X₃ are different.

In some embodiments, X₁ is a IL-12 p40 polypeptide; X₂ is a IL-12 p35 polypeptide; and X₃ is a second fragment of IL-15Rα polypeptide. In some embodiments, X₁ is a IL-12 p35 polypeptide; X₂ is a IL-12 p40 polypeptide; and X₃ is a second fragment of IL-15Rα polypeptide. In some embodiments, X₁ is a IL-15 polypeptide; X₂ is a IL-12 p35 polypeptide; and X₃ is a second fragment of IL-15Rα polypeptide. In some embodiments, X₁ is a IL-15 polypeptide; X₂ is a IL-12 p40 polypeptide; and X₃ is a second fragment of IL-15Rα polypeptide.

In some embodiments, the IL-12 p40 polypeptide comprises an amino acid sequence having of at least, or about, 80%, 85%, 90%, 91%, 92%, 93%, 94%, 95%, 96%, 97%, 98%, or 99% identity to SEQ ID NOs: 1, 2, 3, or 4, or to a IL-12 p40 polypeptide as provided for herein.

In some embodiments, the IL-12 p40 polypeptide comprises an amino acid sequence of SEQ ID NO: 1, 2, 3, or 4, or a IL-12 p40 polypeptide as provided for herein.

In some embodiments, the IL-12 p35 polypeptide comprises an amino acid having of at least, or about, 80%, 85%, 90%, 91%, 92%, 93%, 94%, 95%, 96%, 97%, 98%, or 99% identity to SEQ ID NO: 5, 6, 7, or 8, or to a IL-12 p35 polypeptide as provided for herein. In some embodiments, the IL-12 p35 polypeptide comprises an amino acid sequence of SEQ ID NO: 5, 6, 7, or 8, or a IL-12 p35 polypeptide as provided for herein.

In some embodiments, the IL-15 polypeptide comprises an amino acid sequence having at least, or about, 80%, 85%, 90%, 91%, 92%, 93%, 94%, 95%, 96%, 97%, 98%, or 99% identity to SEQ ID NO: 9 or 10, or to an IL-15 polypeptide as provided for herein. In some embodiments, the IL-15 polypeptide comprises an amino acid sequence of SEQ ID NO: 9 or 10, or an IL-15 polypeptide as provided for herein.

In some embodiments, the IL-15Rα polypeptide comprises an amino acid sequence having of at least, or about, 80%, 85%, 90%, 91%, 92%, 93%, 94%, 95%, 96%, 97%, 98%, or 99% identity to SEQ ID NO: 11, SEQ ID NO: 12, SEQ ID NO: 13, or SEQ ID NO: 14 or to an IL-15Rα polypeptide as provided for herein. In some embodiments, the IL-15Rα polypeptide comprises an amino acid sequence of SEQ ID NO: 11 SEQ ID NO: 12, SEQ ID NO: 13, or SEQ ID NO: 14 or an IL-15Rα polypeptide as provided for herein.

The linkers that are referenced as L1, L2, or L3 are linkers that can be used to link the different polypeptides provided for herein. Although the linkers of L1, L2, and L3 can be the same they can also be different. The structure (e.g., sequence) of each linker can be independent of the structure of the other linkers. Thus, in some embodiments, L1, L2, and L3 are the same or each is different. In some embodiments, L1 and L2 are the same and L3 is different as compared to L1 and L2. In some embodiments, L2 and L3 are the same and L1 is different as compared to L1 and L2. In some embodiments, L1 and L3 are the same and L2 is different as compared to L1 and L3. In some embodiments, they are peptide linkers.

In some embodiments, the peptide linker is a cleavable linker. Without being bound by theory, cleavable linkers comprise at least one cleavage site capable of being recognized and cleaved by an enzyme, which can be referred to a protease. For example, in some embodiments, the enzyme furin recognizes the cleavage site with the general amino acid sequence of RXXR (SEQ ID NO: 69), where X is any amino acid. In some embodiments, the furin cleavage site is RAKR (SEQ ID NO: 70). Other cleavable linkers are known in the art and can also be used in the place of a furin cleavage site. For example, in some embodiments, the linker is a Val-Cit linker, a Val-Gly linker, a Gly-Gly linker, an Ala-Ala-Asn linker, or a linker comprising polyethylene glycol (PEG). Non-limiting examples of cleavable linkers are provided for herein and below.

Accordingly, in some embodiments, one or more of L1, L2, and L3, are each independently, a cleavable linker. In some embodiments at least one of L1, L2, or L3 are a cleavable linker. In some embodiments, L1 is a cleavable linker and L2 and L3 are non-cleavable linkers or not known to be cleavable. In some embodiments, L2 is a cleavable linker and L1 and L3 are non-cleavable linkers or not known to be cleavable. In some embodiments, L2 is a cleavable linker and L1 and L2 are non-cleavable linkers or not known to be cleavable. In some embodiments, the cleavable linker is a furin cleavable linker. In some embodiments, the cleavable linker is a Val-Cit linker, a Val-Gly linker, a Gly-Gly linker, or an Ala-Ala-Asn linker. In some embodiments, the cleavable linker is as illustrated in the table below and can be chosen individually from such table. In some embodiments, one or more of L1, L2, and L3 comprise a sequence of (GSGSGG)n (SEQ ID NO: 16), (GGGGS)n (SEQ ID NO: 17), (GGGGA)n (SEQ ID NO: 18), (GGGSE)n (SEQ ID NO: 19), (GGGSK)n (SEQ ID NO: 20), (AEEEK)n (SEQ ID NO: 21), wherein each n is, independently, from 1 and 5, or a combination thereof. In some embodiments, n is 1. In some embodiments, n is 2. In some embodiments, n is 3. In some embodiments, n is 4. In some embodiments, n is 5. In some embodiments, one or more of L1, L2, and L3 comprises a sequence of GSGSGGGSGSGGGSGSGG (SEQ ID NO: 22). In some embodiments, each of L1, L2, and L3 comprises a sequence of GSGSGGGSGSGGGSGSGG (SEQ ID NO: 22).

Other non-limiting examples of linkers include a glycine/serine linker can be, or comprise, a sequence ofGGGGSGGGGSGGGGSGGGGS (SEQ ID NO: 23) or be, or comprise a sequence of GGGGSGGGGSGGGGS (SEQ ID NO: 24). This is simply a non-limiting example and the linker can have varying number of GGGGS (SEQ ID NO: 25) repeats as provided for herein. In some embodiments, the linker comprises 1, 2, 3, 4, 5, 6, 7, 8, 9, or 10 of the GGGGS (SEQ ID NO: 25) repeats.

In some embodiments, the linker is a flexible linker. In some embodiments, the linker is a rigid linker. In some embodiments, the linker can be as described herein or as illustrated in the following table:

| Type | Sequence | SEQ ID |
|---|---|---|
| flexible | GGGGS | SEQ ID NO: 25 |
| flexible | (GGGGS)₃ | SEQ ID NO: 26 |
| flexible | (GGGGS)ₙ (n = 1, 2, 3, 4) | SEQ ID NO: 27 |
| flexible | GGGGGGGG | SEQ ID NO: 28 |
| flexible | GGGGGG | SEQ ID NO: 29 |
| rigid | (EAAAK)3 | SEQ ID NO: 30 |
| rigid | (EAAK)ₙ (n = 1-3) | SEQ ID NO: 31 |
| rigid | A(EAAAK)₄ALEA(EAAAK)₄A | SEQ ID NO: 32 |
| rigid | AEAAAKEAAAKA | SEQ ID NO: 33 |
| rigid | PAPAP | SEQ ID NO: 34 |
| rigid | (AP)ₙ (10-34 aa) | SEQ ID NO: 35 |
| cleavable | disulfide | n/a |
| cleavable | VSQTSKLTRAETVFPDV | SEQ ID NO: 36 |
| cleavable | PLGLWA | SEQ ID NO: 37 |
| cleavable | RVLAEA | SEQ ID NO: 38 |
| cleavable | EDVVCCSMSY | SEQ ID NO: 39 |
| cleavable | GGIEGRGS | SEQ ID NO: 40 |
| cleavable | TRHRQPRGWE | SEQ ID NO: 41 |
| cleavable | AGNR VRRSVG | SEQ ID NO: 42 |
| cleavable | RRRRRRRRR | SEQ ID NO: 43 |
| cleavable | GFLG | SEQ ID NO: 44 |
| dipeptide | LE | n/a |
| flexible | SGGGSGGGGSGGGGSGGGSGGGSLQ | SEQ ID NO: 45 |
| cleavable | SGGGPGGGGRAKRGGGGPSGGGSLQ | SEQ ID NO: 46 |
| cleavable | SGGGPGGGGRARRGGGGPSGGGSLQ | SEQ ID NO: 47 |
| cleavable | SGGGPGGGGSAGSGGGGPSGGGSLQ | SEQ ID NO: 48 |
| flexible | GSGGSGSGGSGSGGSGSGGS | SEQ ID NO: 49 |
| flexible | SGGGG | SEQ ID NO: 50 |

In some embodiments, the polypeptide can comprise a leader peptide on the N-terminus of the polypeptide. The leader peptide can be used, without being bound to any particular theory, to facilitate in the expression and trafficking of the polypeptide as it is generated by the cell so that, for example, it can be expressed on the surface of the cell. In some embodiments, the leader peptide comprises a sequence of vesicular stomatitis virus G protein (VSV-G). In some embodiments, the leader peptide comprises, consists, or consists essentially of a sequence of MRISKPHLRSISIQCYLCLLLNSHFLTEA (SEQ ID NO: 15).

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 51)
MRISKPHLRSISIQCYLCLLLNSHFLTEAGIHVFILGCF
SAGLPKTEANWVNVISDLKKIEDLIQSMHIDATLYTES
DVHPSCKVTAMKCFLLELQVISLESGDASIHDTVENLII
LANNSLSSNGNVTESGCKECEELEEKNIKEFLQSFVHI
VQMFINTSSGGGPGGGGRAKRGGGGPSGGGSLQIWELKK
DVYVVELDWYPDAPGEMVVLTCDTPEEDGITWTLDQSS
EVLGSGKTLTIQVKEFGDAGQYTCHKGGEVLSHSLLLLH
KKEDGIWSTDILKDQKEPKNKTFLRCEAKNYSGRFTCW
WLTTISTDLTFSVKSSRGSSDPQGVTCGAATLSAERVRG
DNKEYEYSVECQEDSACPAAEESLPIEVMVDAVHKLKY
ENYTSSFFIRDIIKPDPPKNLQLKPLKNSRQVEVSWEYP
DTWSTPHSYFSLTFCVQVQGKSKREKKDRVFTDKTSAT
VICRKNASISVRAQDRYYSSSWSEWASVPCSGSGSGGGS
GSGGGSGSGSRNLPVATPDPGMFPCLHHSQNLLRAVSN
MLQKARQTLEFYPCTSEEIDHEDITKDKTSTVEACLPLE
LTKNESCLNSRETSFITNGSCLASRKTSFMMALCLSSI
YEDLKMYQVEFKTMNAKLLMDPKRQIFLDQNMLAVIDEL
MQALNFNSETVPQKSSLEEPDFYKTKIKLCILLHAFRI
RAVTIDRVMSYLNASGSGGSGSGGSGSGGSITCPPPMSV
EHADIWVKSYSLYSRERYICNSGFKRKAGTSSLTECVL
NKATNVAHWTTPSLKCIRDPALVHQRPAPPSTVTTAGVT
PQPESLSPSGKEPAASSPSSNNTAATTAAIVPGSQLMP
SKSPSTGTTEISSHESSHGTPSQTTAKNWELTASASHQP
PGVYPQGHSDTTVAISTSTVLLCGLSAVSLLACYLKSR
QTPPLASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 52)
MRISKPHLRSISIQCYLCLLLNSHFLTEAGIHVFILGCF
SAGLPKTEANWVNVISDLKKIEDLIQSMHIDATLYTES
DVHPSCKVTAMKCFLLELQVISLESGDASIHDTVENLII
LANNSLSSNGNVTESGCKECEELEEKNIKEFLQSFVHI
VQMFINTSSGGGPGGGGRAKRGGGGPSGGGSLQMWELEK
DVYVVEVDWTPDAPGETVNLTCDTPEEDDITWTSDQRH
GVIGSGKTLTITVKEFLDAGQYTCHKGGETLSHSHLLLH
KKENGIWSTEILKNFKNKTFLKCEAPNYSGRFTCSWLV
QRNMDLKFNIKSSSSSPDSRAVTCGMASLSAEKVTLDQR
DYEKYSVSCQEDVTCPTAEETLPIELALEARQQNKYEN
YSTSFFIRDIIKPDPPKNLQMKPLKNSQVEVSWEYPDSW
STPHSYFSLKFFVRIQRKKEKMKETEEGCNQKGAFLVE
KTSTEVQCKGGNVCVQAQDRYYNSSCSKWACVPCRVRSG
SGSGGGSGSGGGSGSGSRVIPVSGPARCLSQSRNLLKT
TDDMVKTAREKLKHYSCTAEDIDHEDITRDQTSTLKTCL
PLELHKNESCLATRETSSTTRGSCLPPQKTSLMMTLCL
GSIYEDLKMYQTEFQAINAALQNHNHQQIILDKGMLVAI
DELMQSLNHNGETLRQKPPVGEADPYRVKMKLCILLHA
FSTRVVTINRVMGYLSSAGSGGSGSGGSGSGGSITCPPP
MSVEHADIWVKSYSLYSRERYICNSGFKRKAGTSSLTE
CVLNKATNVAHWTTPSLKCIRDPALVHQRPAPPSTVTTA
GVTPQPESLSPSGKEPAASSPSSNNTAATTAAIVPGSQ
LMPSKSPSTGTTEISSHESSHGTPSQTTAKNWELTASAS
HQPPGVYPQGHSDTTVAISTSTVLLCGLSAVSLLACYL
KSRQTPPLASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 53)
MKCLLYLAFLFIGVNCIWELKKDVYVVELDWYPDAPGEM
VVLTCDTPEEDGITWTLDQSSEVLGSGKTLTIQVKEFG
DAGQYTCHKGGEVLSHSLLLLHKKEDGIWSTDILKDQKE
PKNKTFLRCEAKNYSGRFTCWWLTTISTDLTFSVKSSR
GSSDPQGVTCGAATLSAERVRGDNKEYEYSVECQEDSAC
PAAEESLPIEVMVDAVHKLKYENYTSSFFIRDIIKPDP
PKNLQLKPLKNSRQVEVSWEYPDTWSTPHSYFSLTFCVQ
VQGKSKREKKDRVFTDKTSATVICRKNASISVRAQDRY
YSSSWSEWASVPCSGSGSGGGSGSGGGSGSGRNLPVATP
DPGMFPCLHHSQNLLRAVSNMLQKARQTLEFYPCTSEE
IDHEDITKDKTSTVEACLPLELTKNESCLNSRETSFITN
GSCLASRKTSFMMALCLSSIYEDLKMYQVEFKTMNAKL
LMDPKRQIFLDQNMLAVIDELMQALNFNSETVPQKSSLE
EPDFYKTKIKLCILLHAFRIRAVTIDRVMSYLNASGSG
GSGSGGSGSGGSGSGGSGIHVFILGCFSAGLPKTEANWV
NVISDLKKIEDLIQSMHIDATLYTESDVHPSCKVTAMK
CFLLELQVISLESGDASIHDTVENLIILANNSLSSNGNV
TESGCKECEELEEKNIKEFLQSFVHIVQMFINTSSGGG
SGGGGSGGGGSGGGSGGGSLQITCPPPMSVEHADIWVKS
YSLYSRERYICNSGFKRKAGTSSLTECVLNKATNVAHW
TTPSLKCIRDPALVHQRPAPPSTVTTAGVTPQPESLSPS
GKEPAASSPSSNNTAATTAAIVPGSQLMPSKSPSTGTT
EISSHESSHGTPSQTTAKNWELTASASHQPPGVYPQGHS
DTTVAISTSTVLLCGLSAVSLLACYLKSRQTPPLASVE
MEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 54)
MKCLLYLAFLFIGVNCMWELEKDVYVVEVDWTPDAPGETVNLTCDTPEEDDITWTSDQRHGVIGSGKTLTITVKEFL
DAGQYTCHKGGETLSHSHLLLHKKENGIWSTEILKNEKNKTFLKCEAPNYSGRFTCSWLVQRNMDLKFNIKSSSSSP
DSRAVTCGMASLSAEKVTLDQRDYEKYSVSCQEDVTCPTAEETLPIELALEARQQNKYENYSTSFFIRDIIKPDPPK
NLQMKPLKNSQVEVSWEYPDSWSTPHSYFSLKFFVRIQRKKEKMKETEEGCNQKGAFLVEKTSTEVQCKGGNVCVQA
QDRYYNSSCSKWACVPCRVRSGSGSGGGSGSGGGSGSGSRVIPVSGPARCLSQSRNLLKTTDDMVKTAREKLKHYSC
TAEDIDHEDITRDQTSTLKTCLPLELHKNESCLATRETSSTTRGSCLPPQKTSLMMTLCLGSIYEDLKMYQTEFQAI
NAALQNHNHQQIILDKGMLVAIDELMQSLNHNGETLRQKPPVGEADPYRVKMKLCILLHAFSTRVVTINRVMGYLSS
AGSGGSGSGGSGSGGSGSGGSGIHVFILGCFSAGLPKTEANWVNVISDLKKIEDLIQSMHIDATLYTESDVHPSCKV
TAMKCFLLELQVISLESGDASIHDTVENLIILANNSLSSNGNVTESGCKECEELEEKNIKEFLQSFVHIVQMFINTS
SGGGSGGGGSGGGGSGGGSGGGSLQITCPPPMSVEHADIWVKSYSLYSRERYICNSGFKRKAGTSSLTECVLNKATN
VAHWTTPSLKCIRDPALVHQRPAPPSTVTTAGVTPQPESLSPSGKEPAASSPSSNNTAATTAAIVPGSQLMPSKSPS
TGTTEISSHESSHGTPSQTTAKNWELTASASHQPPGVYPQGHSDTTVAISTSTVLLCGLSAVSLLACYLKSRQTPPL
ASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 55)
IWELKKDVYVVELDWYPDAPGEMVVLTCDTPEEDGITWTLDQSSEVLGSGKTLTIQVKEFGDAGQYTCHKGGEVLSH
SLLLLHKKEDGIWSTDILKDQKEPKNKTFLRCEAKNYSGRFTCWWLTTISTDLTFSVKSSRGSSDPQGVTCGAATLS
AERVRGDNKEYEYSVECQEDSACPAAEESLPIEVMVDAVHKLKYENYTSSFFIRDIIKPDPPKNLQLKPLKNSRQVE
VSWEYPDTWSTPHSYFSLTFCVQVQGKSKREKKDRVFTDKTSATVICRKNASISVRAQDRYYSSSWSEWASVPCSGS
GSGGGSGSGGGSGSGRNLPVATPDPGMFPCLHHSQNLLRAVSNMLQKARQTLEFYPCTSEEIDHEDITKDKTSTVEA
CLPLELTKNESCLNSRETSFITNGSCLASRKTSFMMALCLSSIYEDLKMYQVEFKTMNAKLLMDPKRQIFLDQNMLA
VIDELMQALNENSETVPQKSSLEEPDFYKTKIKLCILLHAFRIRAVTIDRVMSYLNASGSGGSGSGGSGSGGSGSGG
SGIHVFILGCFSAGLPKTEANWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTSSGGGSGGGGSGGGGSGGGSG
GGSLQITCPPPMSVEHADIWVKSYSLYSRERYICNSGFKRKAGTSSLTECVLNKATNVAHWTTPSLKCIRDPALVHQ
RPAPPSTVTTAGVTPQPESLSPSGKEPAASSPSSNNTAATTAAIVPGSQLMPSKSPSTGTTEISSHESSHGTPSQTT
AKNWELTASASHQPPGVYPQGHSDTTVAISTSTVLLCGLSAVSLLACYLKSRQTPPLASVEMEAMEALPVTWGTSSR
DEDLENCSHHL

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 56)
MRISKPHLRSISIQCYLCLLLNSHFLTEAGIHVFILGCFSAGLPKTEANWVNVISDLKKIEDLIQSMHIDATLYTES
DVHPSCKVTAMKCFLLELQVISLESGDASIHDTVENLIILANNSLSSNGNVTESGCKECEELEEKNIKEFLQSFVHI
VQMFINTSSGGGSGGGGSGGGGSGGGSGGGSLQITCPPPMSVEHADIWVKSYSLYSRERYICNSGFKRKAGTSSLTE
CVLNKATNVAHWTTPSLKCIRDPALVHQRGSGGSGSGGSGSGGSGSGGSMWELEKDVYVVEVDWTPDAPGETVNLTC
DTPEEDDITWTSDQRHGVIGSGKTLTITVKEFLDAGQYTCHKGGETLSHSHLLLHKKENGIWSTEILKNFKNKTFLK
CEAPNYSGRFTCSWLVQRNMDLKFNIKSSSSSPDSRAVTCGMASLSAEKVTLDQRDYEKYSVSCQEDVTCPTAEETL
PIELALEARQQNKYENYSTSFFIRDIIKPDPPKNLQMKPLKNSQVEVSWEYPDSWSTPHSYFSLKFFVRIQRKKEKM
KETEEGCNQKGAFLVEKTSTEVQCKGGNVCVQAQDRYYNSSCSKWACVPCRVRSGSGSGGGSGSGGGSGSGGRVIPV
SGPARCLSQSRNLLKTTDDMVKTAREKLKHYSCTAEDIDHEDITRDQTSTLKTCLPLELHKNESCLATRETSSTTRG
SCLPPQKTSLMMTLCLGSIYEDLKMYQTEFQAINAALQNHNHQQIILDKGMLVAIDELMQSLNHNGETLRQKPPVGE
ADPYRVKMKLCILLHAFSTRVVTINRVMGYLSSASGGGGPAPPSTVTTAGVTPQPESLSPSGKEPAASSPSSNNTAA
TTAAIVPGSQLMPSKSPSTGTTEISSHESSHGTPSQTTAKNWELTASASHQPPGVYPQGHSDTTVAISTSTVLLCGL
SAVSLLACYLKSRQTPPLASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 57)
MRISKPHLRSISIQCYLCLLLNSHFLTEAGIHVFILGCFSAGLPKTEANWVNVISDLKKIEDLIQSMHIDATLYTES
DVHPSCKVTAMKCFLLELQVISLESGDASIHDTVENLIILANNSLSSNGNVTESGCKECEELEEKNIKEFLQSFVHI
VQMFINTSSGGGSGGGGSGGGGSGGGSGGGSLQITCPPPMSVEHADIWVKSYSLYSRERYICNSGFKRKAGTSSLTE
CVLNKATNVAHWTTPSLKCIRDPALVHQRGSGGSGSGGSGSGGSGSGGSIWELKKDVYVVELDWYPDAPGEMVVLTC
DTPEEDGITWTLDQSSEVLGSGKTLTIQVKEFGDAGQYTCHKGGEVLSHSLLLLHKKEDGIWSTDILKDQKEPKNKT
FLRCEAKNYSGRFTCWWLTTISTDLTFSVKSSRGSSDPQGVTCGAATLSAERVRGDNKEYEYSVECQEDSACPAAEE
SLPIEVMVDAVHKLKYENYTSSFFIRDIIKPDPPKNLQLKPLKNSRQVEVSWEYPDTWSTPHSYFSLTFCVQVQGKS
KREKKDRVFTDKTSATVICRKNASISVRAQDRYYSSSWSEWASVPCSGSGSGGGSGSGGGSGSGGRNLPVATPDPGM
FPCLHHSQNLLRAVSNMLQKARQTLEFYPCTSEEIDHEDITKDKTSTVEACLPLELTKNESCLNSRETSFITNGSCL
ASRKTSFMMALCLSSIYEDLKMYQVEFKTMNAKLLMDPKRQIFLDQNMLAVIDELMQALNENSETVPQKSSLEEPDE
YKTKIKLCILLHAFRIRAVTIDRVMSYLNASSGGGGPAPPSTVTTAGVTPQPESLSPSGKEPAASSPSSNNTAATTA
AIVPGSQLMPSKSPSTGTTEISSHESSHGTPSQTTAKNWELTASASHQPPGVYPQGHSDTTVAISTSTVLLCGLSAV
SLLACYLKSRQTPPLASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 58)
MRISKPHLRSISIQCYLCLLLNSHFLTEAGIHVFILGCFSAGLPKTEANWVNVISDLKKIEDLIQSMHIDATLYTES
DVHPSCKVTAMKCFLLELQVISLESGDASIHDTVENLIILANNSLSSNGNVTESGCKECEELEEKNIKEFLQSFVHI
VQMFINTSSGGGPGGGGSAGSGGGGPSGGGSLQMWELEKDVYVVEVDWTPDAPGETVNLTCDTPEEDDITWTSDQRH
GVIGSGKTLTITVKEFLDAGQYTCHKGGETLSHSHLLLHKKENGIWSTEILKNFKNKTFLKCEAPNYSGRFTCSWLV
QRNMDLKFNIKSSSSSPDSRAVTCGMASLSAEKVTLDQRDYEKYSVSCQEDVTCPTAEETLPIELALEARQQNKYEN
YSTSFFIRDIIKPDPPKNLQMKPLKNSQVEVSWEYPDSWSTPHSYFSLKFFVRIQRKKEKMKETEEGCNQKGAFLVE
KTSTEVQCKGGNVCVQAQDRYYNSSCSKWACVPCRVRSGSGSGGGSGSGGGSGSGSRVIPVSGPARCLSQSRNLLKT
TDDMVKTAREKLKHYSCTAEDIDHEDITRDQTSTLKTCLPLELHKNESCLATRETSSTTRGSCLPPQKTSLMMTLCL
GSIYEDLKMYQTEFQAINAALQNHNHQQIILDKGMLVAIDELMQSLNHNGETLRQKPPVGEADPYRVKMKLCILLHA
FSTRVVTINRVMGYLSSAGSGGSGSGGSGSGGSITCPPPMSVEHADIWVKSYSLYSRERYICNSGFKRKAGTSSLTE
CVLNKATNVAHWTTPSLKCIRDPALVHQRPAPPSTVTTAGVTPQPESLSPSGKEPAASSPSSNNTAATTAAIVPGSQ
LMPSKSPSTGTTEISSHESSHGTPSQTTAKNWELTASASHQPPGVYPQGHSDTTVAISTSTVLLCGLSAVSLLACYL
KSRQTPPLASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 59)
MRISKPHLRSISIQCYLCLLLNSHFLTEAGIHVFILGCFSAGLPKTEANWVNVISDLKKIEDLIQSMHIDATLYTES
DVHPSCKVTAMKCFLLELQVISLESGDASIHDTVENLIILANNSLSSNGNVTESGCKECEELEEKNIKEFLQSFVHI
VQMFINTSSGGGSGGGGSGGGGSGGGSGGGSLQITCPPPMSVEHADIWVKSYSLYSRERYICNSGFKRKAGTSSLTE
CVLNKATNVAHWTTPSLKCIRDPALVHQRPAPPSTVTTAGVTPQPESLSPSGKEPAASSPSSNNTAATTAAIVPGSQ
LMPSKSPSTGTTEISSHESSHGTPSQTTAKNWELTASASHQPPGVYPQGHSDTTVAISTSTVLLCGLSAVSLLACYL
KSRQTPPLASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 60)
MRISKPHLRSISIQCYLCLLLNSHELTEAGIHVFILGCFSAGLPKTEANWVNVISDLKKIEDLIQSMHIDATLYTES
DVHPSCKVTAMKCFLLELQVISLESGDASIHDTVENLIILANNSLSSNGNVTESGCKECEELEEKNIKEFLQSFVHI
VQMFINTSSGGGPGGGGEADEGGGGPSGGGSLQITCPPPMSVEHADIWVKSYSLYSRERYICNSGFKRKAGTSSLTE
CVLNKATNVAHWTTPSLKCIRDPALVHQRPAPPSTVTTAGVTPQPESLSPSGKEPAASSPSSNNTAATTAAIVPGSQ
LMPSKSPSTGTTEISSHESSHGTPSQTTAKNWELTASASHQPPGVYPQGHSDTTVAISTSTVLLCGLSAVSLLACYL
KSRQTPPLASVEMEAMEALPVTWGTSSRDEDLENCSHHL.

In some embodiments, the polypeptide comprises a sequence that is at least 80, 81, 82, 83, 84, 85, 86, 87, 88, 89, 90, 91, 92, 93, 94, 95, 96, 97, 98, 99% identical or is identical to a polypeptide comprising the sequence of:

(SEQ ID NO: 61)
MRISKPHLRSISIQCYLCLLLNSHFLTEAMWELEKDVYVVEVDWTPDAPGETVNLTCDTPEEDDITWTSDQRHGVIG
SGKTLTITVKEFLDAGQYTCHKGGETLSHSHLLLHKKENGIWSTEILKNFKNKTFLKCEAPNYSGRFTCSWLVQRNM
DLKFNIKSSSSSPDSRAVTCGMASLSAEKVTLDQRDYEKYSVSCQEDVTCPTAEETLPIELALEARQQNKYENYSTS
FFIRDIIKPDPPKNLQMKPLKNSQVEVSWEYPDSWSTPHSYFSLKFFVRIQRKKEKMKETEEGCNQKGAFLVEKTST
EVQCKGGNVCVQAQDRYYNSSCSKWACVPCRVRSGSGSGGGSGSGGGSGSGGRVIPVSGPARCLSQSRNLLKTTDDM
VKTAREKLKHYSCTAEDIDHEDITRDQTSTLKTCLPLELHKNESCLATRETSSTTRGSCLPPQKTSLMMTLCLGSIY
EDLKMYQTEFQAINAALQNHNHQQIILDKGMLVAIDELMQSLNHNGETLRQKPPVGEADPYRVKMKLCILLHAFSTR
VVTINRVMGYLSSASGGGGPAPPSTVTTAGVTPQPESLSPSGKEPAASSPSSNNTAATTAAIVPGSQLMPSKSPSTG
TTEISSHESSHGTPSQTTAKNWELTASASHQPPGVYPQGHSDTTVAISTSTVLLCGLSAVSLLACYLKSRQTPPLAS
VEMEAMEALPVTWGTSSRDEDLENCSHHL.

Although the sequences of SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61 are illustrated with specific peptide linkers, these linkers can be modified or replaced with other peptide linkers, such as, but not limited to as those provided for herein.

The polypeptides described herein also encompass variants of the peptides provided for herein. In some embodiments, the polypeptides comprise a sequence of amino acids at least 70%, at least 75%, at least 80%, at least 85%, at least 90%, at least 91%, at least 92%, at least 93% at least 94%, at least 95%, at least 96%, at least 97%, at least 98%, at least 99% substantially similar or identical to the sequences provided for herein. These variants include those that are described herein with the various substitutions described herein and above. In some embodiments, the variant has 1, 2, 3, 4, or 5 additional substitutions. In some embodiments, the substitution is a conservative substitution. In some embodiments, the conservative substitution is selected based upon the following tables:

| | |
|---|---|
| Basic | arginine |
| (positively charged R-groups): | lysine |
| | histidine |
| Acidic | glutamic acid |
| (negatively charged R-groups): | aspartic acid |
| Polar | glutamine |
| (Uncharged R-groups): | asparagine |
| | serine |
| | threonine |
| | cysteine |
| | proline |
| Non-Polar | glycine |
| (aliphatic R-groups): | alanine |
| | valine |
| | methionine |
| | leucine |
| | isoleucine |
| Non-Polar | phenylalanine |
| (aromatic R-groups): | tryptophan |
| | tyrosine |

| Original Residue | Substitutions |
|---|---|
| Ala | Gly; Ser; Thr |
| Arg | Lys; Gln |
| Asn | Gln; His; Ser |
| Asp | Glu; Asn |
| Cys | Ser, Sec |
| Gln | Asn; Ser; Asp; Glu |
| Glu | Asp; Gln; Lys |
| Gly | Ala; Pro; Asn |
| His | Asn; Gln; Tyr; Phe |
| Ile | Leu; Val; Met; Phe |
| Leu | Ile; Val; Met; Phe |
| Lys | Arg; Gln; |
| Met | Leu; Tyr; Ile; norleucine; |
| | Val; Phe |
| Pro | Beta homo proline; Ser; |
| | Thr; Ala; Gly; alpha |
| | homoproline |
| Phe | Met; Leu; Tyr; Trp |
| Ser | Thr; Gly; Asn; Asp |
| Thr | Ser; Asn |
| Trp | Tyr; Phe,; |
| Tyr | Trp; Phe; |
| Val | Ile; Leu; Met; Phe |

The percent identity of two amino acid or two nucleic acid sequences can be determined by visual inspection and mathematical calculation, or for example, the comparison is done by comparing sequence information using a computer program. An exemplary computer program is the Genetics Computer Group (GCG; Madison, Wis.) Wisconsin package version 10.0 program, GAP (Devereux et al. (1984), Nucleic Acids Res. 12: 387-95). The preferred default parameters for the GAP program includes: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14: 6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used.

In some embodiments, a nucleic acid molecule (e.g. DNA or RNA) encoding the a polypeptide provided for herein are provided. In some embodiments, the nucleic acid molecule comprises of a nucleic acid sequence having at least 80%, 85%, 90%, 95%, 96%, 975, 98, 99% or 100% identity to

(SEQ ID NO: 62)
ATGAGGATATCTAAACCTCACCTTCGGAGTATTAGTATCCAATGCTATCTCTGCCTTCTCCTCAATTCACATTTCCT
CACAGAAGCTGGCATACACGTCTTTATTCTTGGATGTTTTTCTGCGGGGTTGCCTAAGACAGAAGCAAATTGGGTAA
ACGTCATCAGTGATTTGAAAAAGATCGAGGATCTCATTCAGAGCATGCACATTGACGCTACGTTGTATACTGAATCC
GACGTTCACCCGTCATGTAAAGTCACTGCCATGAAATGTTTCCTCTTGGAGCTCCAGGTCATCTCCCTGGAAAGCGG
CGACGCTAGTATTCATGATACAGTTGAAAACCTGATCATTCTTGCAAACAATAGCCTCAGTAGTAACGGCAACGTGA
CCGAGTCCGGCTGTAAGGAGTGTGAAGAACTCGAGGAGAAGAACATCAAAGAATTCTTGCAGAGTTTCGTTCATATT
GTGCAAATGTTCATCAACACTTCCTCAGGAGGCGGGCCAGGTGGTGGGGGTAGGGCTAAGAGGGGTGGTGGGGGCCC
AAGTGGGGGCGGCTCCTTGCAGATGTGGGAATTGGAGAAAGACGTTTATGTTGTGGAGGTGGACTGGACTCCAGACG
CGCCCGGTGAGACAGTAAACCTGACTTGCGACACCCCTGAGGAGGACGACATTACATGGACATCAGACCAGAGGCAC
GGGGTTATAGGATCCGGTAAGACCCTCACTATTACCGTGAAAGAGTTCCTTGATGCAGGTCAGTACACTTGTCATAA
GGGAGGGGAAACCCTTTCCCACAGTCACCTTCTGCTGCACAAGAAAGAGAATGGTATATGGTCCACTGAAATCCTTA
AAAATTTTAAGAATAAAACGTTTCTCAAATGTGAGGCCCCAAACTATAGCGGGAGGTTCACGTGCTCTTGGTTGGTC
CAAAGAAATATGGATTTGAAATTCAACATTAAATCATCTAGTAGCTCACCAGACAGTAGGGCTGTGACTTGCGGCAT
GGCATCACTGTCCGCTGAAAAAGTGACCCTTGATCAACGAGACTACGAAAAATATTCTGTCAGCTGCCAGGAGGATG
TGACCTGCCCGACTGCTGAAGAGACGCTGCCCATCGAACTGGCGCTGGAGGCGAGGCAACAGAATAAATATGAAAAT
TACAGTACTTCATTCTTTATACGGGACATTATCAAGCCAGACCCTCCCAAGAACCTCCAAATGAAACCTCTCAAAAA
CAGTCAGGTAGAGGTCTCATGGGAATACCCGGATAGTTGGTCTACACCCCATTCTTATTTCTCCCTTAAATTTTTTG
TTAGGATACAACGGAAGAAGGAGAAGATGAAAGAAACGGAAGAAGGTTGTAACCAGAAGGGCGCATTTCTCGTAGAA
AAAACTTCAACCGAGGTACAATGTAAAGGAGGGAACGTTTGTGTCCAAGCTCAAGATAGGTATTATAACAGCTCATG
TAGCAAGTGGGCGTGCGTCCCGTGTAGGGTTCGCAGTGGATCAGGTAGTGGCGGCGGATCCGGGAGTGGCGGGGGCA
GCGGGTCTGGTTCCCGGGTCATTCCAGTTTCAGGGCCGGCCAGGTGTCTTTCCCAAAGTCGGAACCTTCTCAAGACA
ACGGATGACATGGTCAAGACTGCGAGGGAAAAGCTTAAACACTACTCTTGTACCGCCGAGGACATCGACCATGAAGA
TATTACGCGGGATCAAACCAGTACGTTGAAAACCTGTCTCCCTCTTGAGCTTCATAAGAATGAGTCTTGTCTGGCAA
CGAGGGAAACATCATCAACTACACGAGGTAGTTGCTTGCCGCCTCAGAAGACTTCACTCATGATGACGCTCTGTCTC
GGGTCTATATACGAAGACTTGAAAATGTATCAGACTGAATTTCAGGCTATTAACGCCGCCCTGCAAAATCATAACCA
CCAACAAATTATCCTCGACAAGGGAATGCTCGTTGCAATCGATGAATTGATGCAAAGTTTGAATCATAACGGGGAGA
CACTCCGACAGAAACCACCGGTAGGTGAGGCGGACCCGTACCGAGTGAAAATGAAACTTTGTATCTTGCTCCACGCG
TTCAGCACTCGGGTGGTCACGATAAATCGAGTCATGGGATATTTGTCATCTGCCGGGTCCGGTGGCAGTGGAAGTGG
AGGCAGTGGATCCGGTGGAAGTATAACCTGTCCCCCTCCTATGTCAGTGGAACACGCGGATATCTGGGTGAAGAGTT
ACTCACTGTACTCCAGAGAGCGGTATATCTGTAACTCAGGCTTTAAGCGGAAAGCGGGAACCTCAAGCTTGACTGAG
TGTGTTCTCAACAAAGCAACCAACGTCGCACATTGGACTACTCCGAGTTTGAAGTGTATACGAGATCCGGCATTGGT
TCACCAACGCCCGGCACCACCTAGTACCGTAACCACTGCCGGAGTCACCCCGCAGCCAGAAAGTCTGTCTCCCAGCG
GGAAAGAACCAGCGGCATCATCTCCTTCCTCCAATAATACCGCGGCAACGACTGCCGCAATCGTGCCTGGATCACAG
TTGATGCCCTCTAAGTCCCCATCAACGGGGACAACAGAGATCAGCTCTCATGAGAGCTCACACGGCACTCCGAGCCA
AACTACTGCCAAGAATTGGGAACTTACCGCGTCCGCGAGCCACCAGCCGCCCGGAGTGTACCCTCAAGGCCATTCTG
ACACCACCGTAGCAATTAGCACATCAACTGTGTTGCTCTGCGGTTTGTCCGCCGTTTCACTTCTTGCTTGTTACCTT
AAGAGTCGCCAGACTCCTCCCCTGGCTTCCGTTGAAATGGAGGCTATGGAGGCCCTTCCCGTTACCTGGGGTACCTC
TAGTAGAGATGAGGACCTCGAGAATTGCTCCCATCACCTGTAA.

In some embodiments, a nucleic acid molecule (e.g. DNA or RNA) encoding the a polypeptide provided for herein are provided. In some embodiments, the nucleic acid molecule comprises of a nucleic acid sequence having at least 80%, 85%, 90%, 95%, 96%, 97%, 98%, 99% or 100% identity to

(SEQ ID NO: 63)
ATGAAATGCCTGCTGTATCTGGCGTTTCTGTTTATTGGCGTGAACTGCATTTGGGAACTGAAAAAAGATGTGTATGT
GGTGGAACTGGATTGGTATCCGGATGCGCCGGGCGAAATGGTGGTGCTGACCTGCGATACCCCGGAAGAAGATGGCA
TTACCTGGACCCTGGATCAGAGCAGCGAAGTGCTGGGCAGCGGCAAAACCCTGACCATTCAGGTGAAAGAATTTGGC
GATGCGGGCCAGTATACCTGCCATAAAGGCGGCGAAGTGCTGAGCCATAGCCTGCTGCTGCTGCATAAAAAAGAAGA
TGGCATTTGGAGCACCGATATTCTGAAAGATCAGAAAGAACCGAAAAACAAAACCTTTCTGCGCTGCGAAGCGAAAA
ACTATAGCGGCCGCTTTACCTGCTGGTGGCTGACCACCATTAGCACCGATCTGACCTTTAGCGTGAAAAGCAGCCGC
GGCAGCAGCGATCCGCAGGGCGTGACCTGCGGCGCGGCGACCCTGAGCGCGGAACGCGTGCGCGGCGATAACAAAGA
ATATGAATATAGCGTGGAATGCCAGGAAGATAGCGCGTGCCCGGCGGCGGAAGAAAGCCTGCCGATTGAAGTGATGG
TGGATGCGGTGCATAAACTGAAATATGAAAACTATACCAGCAGCTTTTTTATTCGCGATATTATTAAACCGGATCCG
CCGAAAAACCTGCAGCTGAAACCGCTGAAAAACAGCCGCCAGGTGGAAGTGAGCTGGGAATATCCGGATACCTGGAG
CACCCCGCATAGCTATTTTAGCCTGACCTTTTGCGTGCAGGTGCAGGGCAAAAGCAAACGCGAAAAAAAAGATCGCG
TGTTTACCGATAAAACCAGCGCGACCGTGATTTGCCGCAAAAACGCGAGCATTAGCGTGCGCGCGCAGGATCGCTAT
TATAGCAGCAGCTGGAGCGAATGGGCGAGCGTGCCGTGCAGCGGCAGCGGCAGCGGCGGCGGCAGCGGCAGCGGCGG
CGGCAGCGGCAGCGGCCGCAACCTGCCGGTGGCGACCCCGGATCCGGGCATGTTTCCGTGCCTGCATCATAGCCAGA
ACCTGCTGCGCGCGGTGAGCAACATGCTGCAGAAAGCGCGCCAGACCCTGGAATTTTATCCGTGCACCAGCGAAGAA
ATTGATCATGAAGATATTACCAAAGATAAAACCAGCACCGTGGAAGCGTGCCTGCCGCTGGAACTGACCAAAAACGA
AAGCTGCCTGAACAGCCGCGAAACCAGCTTTATTACCAACGGCAGCTGCCTGGCGAGCCGCAAAACCAGCTTTATGA
TGGCGCTGTGCCTGAGCAGCATTTATGAAGATCTGAAAATGTATCAGGTGGAATTTAAAACCATGAACGCGAAACTG
CTGATGGATCCGAAACGCCAGATTTTTCTGGATCAGAACATGCTGGCGGTGATTGATGAACTGATGCAGGCGCTGAA
CTTTAACAGCGAAACCGTGCCGCAGAAAAGCAGCCTGGAAGAACCGGATTTTTATAAAACCAAAATTAAACTGTGCA
TTCTGCTGCATGCGTTTCGCATTCGCGCGGTGACCATTGATCGCGTGATGAGCTATCTGAACGCGAGCGGCAGCGGC
GGCAGCGGCAGCGGCGGCAGCGGCAGCGGCGGCAGCGGCAGCGGCGGCAGCGGCATTCATGTGTTTATTCTGGGCTG
CTTTAGCGCGGGCCTGCCGAAAACCGAAGCGAACTGGGTGAACGTGATTAGCGATCTGAAAAAAATTGAAGATCTGA
TTCAGAGCATGCATATTGATGCGACCCTGTATACCGAAAGCGATGTGCATCCGAGCTGCAAAGTGACCGCGATGAAA
TGCTTTCTGCTGGAACTGCAGGTGATTAGCCTGGAAAGCGGCGATGCGAGCATTCATGATACCGTGGAAAACCTGAT
TATTCTGGCGAACAACAGCCTGAGCAGCAACGGCAACGTGACCGAAAGCGGCTGCAAAGAATGCGAAGAACTGGAAG
AAAAAAACATTAAAGAATTTCTGCAGAGCTTTGTGCATATTGTGCAGATGTTTATTAACACCAGCAGCGGCGGCGGC
AGCGGCGGCGGCGGCAGCGGCGGCGGCGGCAGCGGCGGCGGCAGCGGCGGCGGCAGCCTGCAGATTACCTGCCCGCC
GCCGATGAGCGTGGAACATGCGGATATTTGGGTGAAAAGCTATAGCCTGTATAGCCGCGAACGCTATATTTGCAACA
GCGGCTTTAAACGCAAAGCGGGCACCAGCAGCCTGACCGAATGCGTGCTGAACAAAGCGACCAACGTGGCGCATTGG
ACCACCCCGAGCCTGAAATGCATTCGCGATCCGGCGCTGGTGCATCAGCGCCCGGCGCCGCCGAGCACCGTGACCAC
CGCGGGCGTGACCCCGCAGCCGGAAAGCCTGAGCCCGAGCGGCAAAGAACCGGCGGCGAGCAGCCCGAGCAGCAACA
ACACCGCGGCGACCACCGCGGCGATTGTGCCGGGCAGCCAGCTGATGCCGAGCAAAAGCCCGAGCACCGGCACCACC
GAAATTAGCAGCCATGAAAGCAGCCATGGCACCCCGAGCCAGACCACCGCGAAAAACTGGGAACTGACCGCGAGCGC
GAGCCATCAGCCGCCGGGCGTGTATCCGCAGGGCCATAGCGATACCACCGTGGCGATTAGCACCAGCACCGTGCTGC
TGTGCGGCCTGAGCGCGGTGAGCCTGCTGGCGTGCTATCTGAAAAGCCGCCAGACCCCGCCGCTGGCGAGCGTGGAA
ATGGAAGCGATGGAAGCGCTGCCGGTGACCTGGGGCACCAGCAGCCGCGATGAAGATCTGGAAAACTGCAGCCATCA
TCTGTAA.

In some embodiments, a nucleic acid molecule (e.g. DNA or RNA) encoding the a polypeptide provided for herein are provided. In some embodiments, the nucleic acid molecule comprises of a nucleic acid sequence having at least 80%, 85%, 90%, 95%, 96%, 97%, 98%, 99% or 100% identity to

(SEQ ID NO: 64)
ATGAGAATCTCTAAACCACACTTGCGCTCCATCAGTATACAATGCTATCTGTGTCTGCTCCTTAACTCACACTTCCT
TACCGAAGCGGGTATCCACGTTTTCATACTCGGGTGCTTCTCCGCTGGGTTGCCCAAAACAGAGGCTAACTGGGTGA
ATGTTATCAGCGACCTCAAAAAGATCGAGGACTTGATACAGAGTATGCATATAGATGCCACCCTTTATACCGAGAGT
GATGTTCATCCGAGTTGTAAGGTAACTGCTATGAAGTGTTTTCTGCTTGAACTTCAGGTAATAAGCCTTGAATCCGG
AGACGCAAGCATTCATGATACGGTGGAAAACCTGATTATTCTCGCTAACAACTCCCTTTCAAGTAATGGCAACGTAA
CCGAATCTGGCTGTAAAGAGTGCGAGGAGCTTGAAGAGAAAAACATCAAAGAGTTTTTGCAATCCTTTGTTCATATT
GTTCAAATGTTTATAAACACGTCATCAGGCGGAGGCAGCGGAGGCGGTGGCAGTGGAGGAGGAGGATCTGGAGGCGG
ATCCGGAGGAGGGAGTCTTCAGATCACGTGCCCACCGCCAATGAGTGTCGAGCACGCGGATATCTGGGTTAAATCCT
ATAGCCTCTACTCCCGGGAGAGGTACATATGCAACAGCGGCTTTAAACGAAAAGCGGGAACCTCCTCACTCACGGAG
TGCGTCTTGAACAAAGCGACTAACGTGGCACACTGGACTACTCCTTCTCTCAAATGCATCCGAGACCCTGCGCTGGT
ACATCAGAGAGGAAGTGGGGGCAGTGGAAGCGGAGGAAGTGGCAGCGGCGGATCTGGCTCTGGTGGGAGTATGTGGG
AATTGGAGAAGGACGTATATGTGGTGGAGGTAGACTGGACCCCAGATGCTCCGGGAGAGACCGTCAATCTTACATGC
GACACTCCAGAGGAAGATGACATAACTTGGACATCTGACCAACGCCATGGTGTGATTGGATCCGGAAAAACCCTCAC
GATTACGGTCAAGGAATTTCTGGATGCAGGCCAGTATACTTGCCACAAGGGAGGAGAAACGTTGTCACATTCTCATC
TTTTGCTTCACAAGAAAGAAAATGGTATCTGGTCTACAGAAATCCTCAAGAACTTTAAGAATAAAACCTTCCTGAAA
TGCGAAGCTCCCAATTACAGTGGGCGATTTACATGTTCTTGGCTTGTACAAAGGAACATGGACTTGAAGTTTAATAT
TAAGAGCAGTAGCTCATCACCGGACTCACGAGCTGTAACATGTGGAATGGCGTCACTGTCCGCAGAGAAGGTAACGC
TTGACCAGCGAGATTACGAAAAATACAGCGTTAGTTGTCAAGAAGATGTCACTTGTCCTACGGCGGAGGAGACACTT
CCAATTGAGCTTGCATTGGAAGCTCGACAGCAGAACAAATATGAAAACTACAGTACGAGTTTCTTTATTAGGGACAT
AATTAAGCCGGATCCCCCTAAGAATTTGCAGATGAAACCTCTGAAAAACAGCCAGGTCGAAGTTAGCTGGGAATATC
CTGACTCTTGGTCAACCCCCCACTCATACTTCTCTCTGAAGTTCTTTGTGAGGATTCAGCGGAAGAAAGAAAAAATG
AAGGAAACTGAGGAAGGGTGCAATCAAAAAGGTGCCTTTCTGGTGGAGAAAACGTCTACGGAGGTACAGTGTAAGGG
TGGAAATGTCTGTGTTCAAGCGCAAGATAGATATTACAACTCATCCTGCTCTAAGTGGGCCTGCGTCCCCTGCCGGG
TAAGGAGTGGTTCTGGGTCTGGCGGTGGATCTGGGAGTGGCGGCGGCAGTGGCAGTGGAGGGCGGGTAATTCCGGTG
AGTGGTCCCGCTCGATGTCTGTCCCAATCCAGAAACTTGCTCAAGACCACTGACGATATGGTTAAGACGGCCCGGGA
GAAACTGAAGCACTACTCCTGTACAGCAGAAGACATTGACCATGAAGATATCACGAGAGATCAAACATCCACTCTTA
AAACGTGTTTGCCGCTGGAGCTTCATAAAAACGAGTCTTGTTTGGCTACACGCGAGACCTCCAGTACTACGCGAGGT
AGTTGTCTTCCACCACAAAAGACTAGTCTCATGATGACGTTGTGTTTGGGTTCTATCTACGAAGACCTCAAGATGTA
TCAAACTGAATTTCAGGCAATTAATGCCGCCCTGCAAAATCATAATCATCAACAGATCATTCTTGATAAGGGGATGC
TCGTTGCAATTGATGAACTCATGCAATCACTTAATCACAACGGTGAGACGTTGCGACAGAAACCACCAGTTGGTGAG
GCCGACCCGTATCGAGTCAAAATGAAACTTTGTATACTCTTGCATGCATTCAGTACGCGAGTAGTGACAATTAATAG
GGTCATGGGCTATCTTAGCAGCGCATCCGGCGGAGGCGGACCAGCTCCGCCCAGTACAGTAACTACGGCCGGGGTTA
CGCCCCAACCAGAGAGTCTCAGTCCAAGTGGTAAAGAACCTGCGGCGAGTAGCCCGAGCTCCAACAACACCGCCGCG
ACTACAGCAGCGATTGTTCCGGGGAGCCAGTTGATGCCCAGCAAGTCTCCTAGTACTGGCACAACTGAAATATCAAG
TCACGAAAGCTCTCATGGGACTCCTTCCCAGACAACGGCTAAGAACTGGGAGTTGACAGCAAGTGCGTCACATCAAC
CACCAGGCGTCTACCCACAAGGCCATTCTGACACTACTGTCGCCATTAGTACGTCCACCGTTTTGTTGTGTGGCCTC
AGTGCTGTGAGTTTGTTGGCCTGTTACCTGAAAAGTAGACAGACTCCACCATTGGCGTCTGTTGAGATGGAGGCCAT
GGAGGCGCTCCCGGTGACCTGGGGTACGTCCAGCCGCGATGAAGACCTTGAGAATTGTAGTCATCATTTGTAA.

In some embodiments, a nucleic acid molecule (e.g. DNA or RNA) encoding the a polypeptide provided for herein are provided. In some embodiments, the nucleic acid molecule comprises of a nucleic acid sequence having at least 80%, 85%, 90%, 95%, 96%, 97%, 98%, 99% or 100% identity to

(SEQ ID NO: 65)
ATGAGGATATCTAAACCTCACCTTCGGAGTATTAGTATCCAATGCTATCTCTGCCTTCTCCTCAATTCACATTTCCT
CACAGAAGCTGGCATACACGTCTTTATTCTTGGATGTTTTTCTGCGGGGTTGCCTAAGACAGAAGCAAATTGGGTAA
ACGTCATCAGTGATTTGAAAAAGATCGAGGATCTCATTCAGAGCATGCACATTGACGCTACGTTGTATACTGAATCC
GACGTTCACCCGTCATGTAAAGTCACTGCCATGAAATGTTTCCTCTTGGAGCTCCAGGTCATCTCCCTGGAAAGCGG
CGACGCTAGTATTCATGATACAGTTGAAAACCTGATCATTCTTGCAAACAATAGCCTCAGTAGTAACGGCAACGTGA
CCGAGTCCGGCTGTAAGGAGTGTGAAGAACTCGAGGAGAAGAACATCAAAGAATTCTTGCAGAGTTTCGTTCATATT
GTGCAAATGTTCATCAACACTTCCTCAGGAGGCGGGCCAGGTGGTGGGGGTAGTGCTGGTTCCGGTGGTGGGGGCCC
AAGTGGGGGCGGCTCCTTGCAGATGTGGGAATTGGAGAAAGACGTTTATGTTGTGGAGGTGGACTGGACTCCAGACG
CGCCCGGTGAGACAGTAAACCTGACTTGCGACACCCCTGAGGAGGACGACATTACATGGACATCAGACCAGAGGCAC
GGGGTTATAGGATCCGGTAAGACCCTCACTATTACCGTGAAAGAGTTCCTTGATGCAGGTCAGTACACTTGTCATAA
GGGAGGGGAAACCCTTTCCCACAGTCACCTTCTGCTGCACAAGAAAGAGAATGGTATATGGTCCACTGAAATCCTTA
AAAATTTTAAGAATAAAACGTTTCTCAAATGTGAGGCCCCAAACTATAGCGGGAGGTTCACGTGCTCTTGGTTGGTC
CAAAGAAATATGGATTTGAAATTCAACATTAAATCATCTAGTAGCTCACCAGACAGTAGGGCTGTGACTTGCGGCAT
GGCATCACTGTCCGCTGAAAAAGTGACCCTTGATCAACGAGACTACGAAAAATATTCTGTCAGCTGCCAGGAGGATG
TGACCTGCCCGACTGCTGAAGAGACGCTGCCCATCGAACTGGCGCTGGAGGCGAGGCAACAGAATAAATATGAAAAT
TACAGTACTTCATTCTTTATACGGGACATTATCAAGCCAGACCCTCCCAAGAACCTCCAAATGAAACCTCTCAAAAA
CAGTCAGGTAGAGGTCTCATGGGAATACCCGGATAGTTGGTCTACACCCCATTCTTATTTCTCCCTTAAATTTTTTG
TTAGGATACAACGGAAGAAGGAGAAGATGAAAGAAACGGAAGAAGGTTGTAACCAGAAGGGCGCATTTCTCGTAGAA
AAAACTTCAACCGAGGTACAATGTAAAGGAGGGAACGTTTGTGTCCAAGCTCAAGATAGGTATTATAACAGCTCATG
TAGCAAGTGGGCGTGCGTCCCGTGTAGGGTTCGCAGTGGATCAGGTAGTGGCGGCGGATCCGGGAGTGGCGGGGGCA
GCGGGTCTGGTTCCCGGGTCATTCCAGTTTCAGGGCCGGCCAGGTGTCTTTCCCAAAGTCGGAACCTTCTCAAGACA
ACGGATGACATGGTCAAGACTGCGAGGGAAAAGCTTAAACACTACTCTTGTACCGCCGAGGACATCGACCATGAAGA
TATTACGCGGGATCAAACCAGTACGTTGAAAACCTGTCTCCCTCTTGAGCTTCATAAGAATGAGTCTTGTCTGGCAA
CGAGGGAAACATCATCAACTACACGAGGTAGTTGCTTGCCGCCTCAGAAGACTTCACTCATGATGACGCTCTGTCTC
GGGTCTATATACGAAGACTTGAAAATGTATCAGACTGAATTTCAGGCTATTAACGCCGCCCTGCAAAATCATAACCA
CCAACAAATTATCCTCGACAAGGGAATGCTCGTTGCAATCGATGAATTGATGCAAAGTTTGAATCATAACGGGGAGA
CACTCCGACAGAAACCACCGGTAGGTGAGGCGGACCCGTACCGAGTGAAAATGAAACTTTGTATCTTGCTCCACGCG
TTCAGCACTCGGGTGGTCACGATAAATCGAGTCATGGGATATTTGTCATCTGCCGGGTCCGGTGGCAGTGGAAGTGG
AGGCAGTGGATCCGGTGGAAGTATAACCTGTCCCCCTCCTATGTCAGTGGAACACGCGGATATCTGGGTGAAGAGTT
ACTCACTGTACTCCAGAGAGCGGTATATCTGTAACTCAGGCTTTAAGCGGAAAGCGGGAACCTCAAGCTTGACTGAG
TGTGTTCTCAACAAAGCAACCAACGTCGCACATTGGACTACTCCGAGTTTGAAGTGTATACGAGATCCGGCATTGGT
TCACCAACGCCCGGCACCACCTAGTACCGTAACCACTGCCGGAGTCACCCCGCAGCCAGAAAGTCTGTCTCCCAGCG
GGAAAGAACCAGCGGCATCATCTCCTTCCTCCAATAATACCGCGGCAACGACTGCCGCAATCGTGCCTGGATCACAG
TTGATGCCCTCTAAGTCCCCATCAACGGGGACAACAGAGATCAGCTCTCATGAGAGCTCACACGGCACTCCGAGCCA
AACTACTGCCAAGAATTGGGAACTTACCGCGTCCGCGAGCCACCAGCCGCCCGGAGTGTACCCTCAAGGCCATTCTG
ACACCACCGTAGCAATTAGCACATCAACTGTGTTGCTCTGCGGTTTGTCCGCCGTTTCACTTCTTGCTTGTTACCTT
AAGAGTCGCCAGACTCCTCCCCTGGCTTCCGTTGAAATGGAGGCTATGGAGGCCCTTCCCGTTACCTGGGGTACCTC
TAGTAGAGATGAGGACCTCGAGAATTGCTCCCATCACCTGTAA.

In some embodiments, a nucleic acid molecule (e.g. DNA or RNA) encoding the a polypeptide provided for herein are provided. In some embodiments, the nucleic acid molecule comprises of a nucleic acid sequence having at least 80%, 85%, 90%, 95%, 96%, 97%, 98%, 99% or 100% identity to

(SEQ ID NO: 66)
ATGAGAATCTCTAAACCACACTTGCGCTCCATCAGTATACAATGCTATCTGTGTCTGCTCCTTAACTCACACTTCCT
TACCGAAGCGGGTATCCACGTTTTCATACTCGGGTGCTTCTCCGCTGGGTTGCCCAAAACAGAGGCTAACTGGGTGA
ATGTTATCAGCGACCTCAAAAAGATCGAGGACTTGATACAGAGTATGCATATAGATGCCACCCTTTATACCGAGAGT
GATGTTCATCCGAGTTGTAAGGTAACTGCTATGAAGTGTTTTCTGCTTGAACTTCAGGTAATAAGCCTTGAATCCGG
AGACGCAAGCATTCATGATACGGTGGAAAACCTGATTATTCTCGCTAACAACTCCCTTTCAAGTAATGGCAACGTAA
CCGAATCTGGCTGTAAAGAGTGCGAGGAGCTTGAAGAGAAAAACATCAAAGAGTTTTTGCAATCCTTTGTTCATATT
GTTCAAATGTTTATAAACACGTCATCAGGCGGAGGCAGCGGAGGCGGTGGCAGTGGAGGAGGAGGATCTGGAGGCGG
ATCCGGAGGAGGGAGTCTTCAGATCACGTGCCCACCGCCAATGAGTGTCGAGCACGCGGATATCTGGGTTAAATCCT
ATAGCCTCTACTCCCGGGAGAGGTACATATGCAACAGCGGCTTTAAACGAAAAGCGGGAACCTCCTCACTCACGGAG
TGCGTCTTGAACAAAGCGACTAACGTGGCACACTGGACTACTCCTTCTCTCAAATGCATCCGAGACCCTGCGCTGGT
ACATCAGAGACCAGCTCCGCCCAGTACAGTAACTACGGCCGGGGTTACGCCCCAACCAGAGAGTCTCAGTCCAAGTG
GTAAAGAACCTGCGGCGAGTAGCCCGAGCTCCAACAACACCGCCGCGACTACAGCAGCGATTGTTCCGGGGAGCCAG
TTGATGCCCAGCAAGTCTCCTAGTACTGGCACAACTGAAATATCAAGTCACGAAAGCTCTCATGGGACTCCTTCCCA
GACAACGGCTAAGAACTGGGAGTTGACAGCAAGTGCGTCACATCAACCACCAGGCGTCTACCCACAAGGCCATTCTG
ACACTACTGTCGCCATTAGTACGTCCACCGTTTTGTTGTGTGGCCTCAGTGCTGTGAGTTTGTTGGCCTGTTACCTG
AAAAGTAGACAGACTCCACCATTGGCGTCTGTTGAGATGGAGGCCATGGAGGCGCTCCCGGTGACCTGGGGTACGTC
CAGCCGCGATGAAGACCTTGAGAATTGTAGTCATCATTTGTAA.

In some embodiments, a nucleic acid molecule (e.g. DNA or RNA) encoding the a polypeptide provided for herein are provided. In some embodiments, the nucleic acid molecule comprises of a nucleic acid sequence having at least 80%, 85%, 90%, 95%, 96%, 97%, 98%, 99% or 100% identity to

(SEQ ID NO: 67)
ATGAGGATATCTAAACCTCACCTTCGGAGTATTAGTATCCAATGCTATCTCTGCCTTCTCCTCAATTCACATTTCCT
CACAGAAGCTGGCATACACGTCTTTATTCTTGGATGTTTTTCTGCGGGGTTGCCTAAGACAGAAGCAAATTGGGTAA
ACGTCATCAGTGATTTGAAAAAGATCGAGGATCTCATTCAGAGCATGCACATTGACGCTACGTTGTATACTGAATCC
GACGTTCACCCGTCATGTAAAGTCACTGCCATGAAATGTTTCCTCTTGGAGCTCCAGGTCATCTCCCTGGAAAGCGG
CGACGCTAGTATTCATGATACAGTTGAAAACCTGATCATTCTTGCAAACAATAGCCTCAGTAGTAACGGCAACGTGA
CCGAGTCCGGCTGTAAGGAGTGTGAAGAACTCGAGGAGAAGAACATCAAAGAATTCTTGCAGAGTTTCGTTCATATT
GTGCAAATGTTCATCAACACTTCCTCAGGAGGGGGCCAGGTGGTGGGGGTGAAGCTGACGAGGGTGGTGGGGGCCC
AAGTGGGGGCGGCTCCTTGCAGATAACCTGTCCCCCTCCTATGTCAGTGGAACACGCGGATATCTGGGTGAAGAGTT
ACTCACTGTACTCCAGAGAGCGGTATATCTGTAACTCAGGCTTTAAGCGGAAAGCGGGAACCTCAAGCTTGACTGAG
TGTGTTCTCAACAAAGCAACCAACGTCGCACATTGGACTACTCCGAGTTTGAAGTGTATACGAGATCCGGCATTGGT
TCACCAACGCCCGGCACCACCTAGTACCGTAACCACTGCCGGAGTCACCCCGCAGCCAGAAAGTCTGTCTCCCAGCG
GGAAAGAACCAGCGGCATCATCTCCTTCCTCCAATAATACCGCGGCAACGACTGCCGCAATCGTGCCTGGATCACAG
TTGATGCCCTCTAAGTCCCCATCAACGGGGACAACAGAGATCAGCTCTCATGAGAGCTCACACGGCACTCCGAGCCA
AACTACTGCCAAGAATTGGGAACTTACCGCGTCCGCGAGCCACCAGCCGCCCGGAGTGTACCCTCAAGGCCATTCTG
ACACCACCGTAGCAATTAGCACATCAACTGTGTTGCTCTGCGGTTTGTCCGCCGTTTCACTTCTTGCTTGTTACCTT
AAGAGTCGCCAGACTCCTCCCCTGGCTTCCGTTGAAATGGAGGCTATGGAGGCCCTTCCCGTTACCTGGGGTACCTC
TAGTAGAGATGAGGACCTCGAGAATTGCTCCCATCACCTGTAA.

In some embodiments, a nucleic acid molecule (e.g. DNA or RNA) encoding the a polypeptide provided for herein are provided. In some embodiments, the nucleic acid molecule comprises of a nucleic acid sequence having at least 80%, 85%, 90%, 95%, 96%, 97%, 98%, 99% or 100% identity to

(SEQ ID NO: 68)
ATGAGAATCTCTAAACCACACTTGCGCTCCATCAGTATACAATGCTATCTGTGTCTGCTCCTTAACTCACACTTCCT
TACCGAAGCGATGTGGGAATTGGAGAAGGACGTATATGTGGTGGAGGTAGACTGGACCCCAGATGCTCCGGGAGAGA
CCGTCAATCTTACATGCGACACTCCAGAGGAAGATGACATAACTTGGACATCTGACCAACGCCATGGTGTGATTGGA
TCCGGAAAAACCCTCACGATTACGGTCAAGGAATTTCTGGATGCAGGCCAGTATACTTGCCACAAGGGAGGAGAAAC
GTTGTCACATTCTCATCTTTTGCTTCACAAGAAAGAAAATGGTATCTGGTCTACAGAAATCCTCAAGAACTTTAAGA
ATAAAACCTTCCTGAAATGCGAAGCTCCCAATTACAGTGGGCGATTTACATGTTCTTGGCTTGTACAAAGGAACATG
GACTTGAAGTTTAATATTAAGAGCAGTAGCTCATCACCGGACTCACGAGCTGTAACATGTGGAATGGCGTCACTGTC
CGCAGAGAAGGTAACGCTTGACCAGCGAGATTACGAAAAATACAGCGTTAGTTGTCAAGAAGATGTCACTTGTCCTA
CGGCGGAGGAGACACTTCCAATTGAGCTTGCATTGGAAGCTCGACAGCAGAACAAATATGAAAACTACAGTACGAGT
TTCTTTATTAGGGACATAATTAAGCCGGATCCCCCTAAGAATTTGCAGATGAAACCTCTGAAAAACAGCCAGGTCGA
AGTTAGCTGGGAATATCCTGACTCTTGGTCAACCCCCCACTCATACTTCTCTCTGAAGTTCTTTGTGAGGATTCAGC
GGAAGAAAGAAAAAATGAAGGAAACTGAGGAAGGGTGCAATCAAAAAGGTGCCTTTCTGGTGGAGAAAACGTCTACG
GAGGTACAGTGTAAGGGTGGAAATGTCTGTGTTCAAGCGCAAGATAGATATTACAACTCATCCTGCTCTAAGTGGGC
CTGCGTCCCCTGCCGGGTAAGGAGTGGTTCTGGGTCTGGCGGTGGATCTGGGAGTGGCGGCGGCAGTGGCAGTGGAG
GGCGGGTAATTCCGGTGAGTGGTCCCGCTCGATGTCTGTCCCAATCCAGAAACTTGCTCAAGACCACTGACGATATG
GTTAAGACGGCCCGGGAGAAACTGAAGCACTACTCCTGTACAGCAGAAGACATTGACCATGAAGATATCACGAGAGA
TCAAACATCCACTCTTAAAACGTGTTTGCCGCTGGAGCTTCATAAAAACGAGTCTTGTTTGGCTACACGCGAGACCT
CCAGTACTACGCGAGGTAGTTGTCTTCCACCACAAAAGACTAGTCTCATGATGACGTTGTGTTTGGGTTCTATCTAC
GAAGACCTCAAGATGTATCAAACTGAATTTCAGGCAATTAATGCCGCCCTGCAAAATCATAATCATCAACAGATCAT
TCTTGATAAGGGGATGCTCGTTGCAATTGATGAACTCATGCAATCACTTAATCACAACGGTGAGACGTTGCGACAGA
AACCACCAGTTGGTGAGGCCGACCCGTATCGAGTCAAAATGAAACTTTGTATACTCTTGCATGCATTCAGTACGCGA
GTAGTGACAATTAATAGGGTCATGGGCTATCTTAGCAGCGCATCCGGCGGAGGCGGACCAGCTCCGCCCAGTACAGT
AACTACGGCCGGGGTTACGCCCCAACCAGAGAGTCTCAGTCCAAGTGGTAAAGAACCTGCGGCGAGTAGCCCGAGCT
CCAACAACACCGCCGCGACTACAGCAGCGATTGTTCCGGGGAGCCAGTTGATGCCCAGCAAGTCTCCTAGTACTGGC
ACAACTGAAATATCAAGTCACGAAAGCTCTCATGGGACTCCTTCCCAGACAACGGCTAAGAACTGGGAGTTGACAGC
AAGTGCGTCACATCAACCACCAGGCGTCTACCCACAAGGCCATTCTGACACTACTGTCGCCATTAGTACGTCCACCG
TTTTGTTGTGTGGCCTCAGTGCTGTGAGTTTGTTGGCCTGTTACCTGAAAAGTAGACAGACTCCACCATTGGCGTCT
GTTGAGATGGAGGCCATGGAGGCGCTCCCGGTGACCTGGGGTACGTCCAGCCGCGATGAAGACCTTGAGAATTGTAG
TCATCATTTGTAA.

The sequence of SEQ ID NO: 62, SEQ ID NO: 63, SEQ ID NO: 64, SEQ ID NO: 65, SEQ ID NO: 66, SEQ ID NO: 67, and SEQ ID NO: 68 are merely exemplary sequences that encode for polypeptides described herein. Due to the degenerate nature of codons other nucleic acid molecules can be used. In some embodiments, the nucleic acid molecule is codon optimized for expression in a bacterial system. In some embodiments, the nucleic acid molecule is codon optimized for expression in an eukaryotic system or cell. In some embodiments, the nucleic acid molecule is a DNA or RNA molecule that encodes a polypeptide as provided for herein. In some embodiments, the RNA molecule is a mRNA molecule.

The nucleic acid molecule can be prepared by synthesis or other traditional techniques known to one of skill in the art once provided a sequence, which can be either the sequence of the polypeptide that is to be encoded by the nucleic acid molecule or the nucleic acid sequence itself.

Another method of constructing a DNA sequence encoding a polypeptide as provided for herein would be chemical synthesis. This for example includes direct synthesis of a peptide by chemical means of the protein sequence encoding for a polypeptide as provided for herein. This method may incorporate both natural and unnatural amino acids at various positions. Alternatively, a nucleic acid molecule which encodes a desired protein may be synthesized by chemical means using an oligonucleotide synthesizer. The oligonucleotides are designed based on the amino acid sequence of the desired protein, which can also be selected by using codons that are favored in the cell in which the recombinant variant will be produced. It is well recognized that the genetic code is degenerate, i.e., that an amino acid may be coded for by more than one codon. Accordingly, it will be appreciated that for a given DNA sequence encoding a particular polypeptide as provided for herein there will be many DNA degenerate sequences that will code for that polypeptide. Accordingly, in some embodiments, a nucleic acid molecule is provided that encodes the polypeptides provided for herein. The nucleic acid molecule can be DNA or RNA.

In some embodiments, the nucleic acid molecule will encode a signal sequence or leader peptide sequence, such as provided for herein. A signal sequence can be chosen based upon the cell that will be expressed in. In some embodiments, if the host cell is prokaryotic, the nucleic acid molecule does not comprise a signal sequence. In some embodiments, if the host cell is a eukaryotic cell, the signal sequence can be used. In some embodiments, the signal sequence or leader sequence is as provided for herein. The signal or leader sequence of the protein can also be from the immature proteins.

“Recombinant” as it applies to polypeptides or proteins, means that the production of the protein is dependent on at least one step in which nucleic acids, which may or may not encode the protein, are introduced into a cell in which they are not naturally found. The nucleic acid molecule can also be referred to as a heterologous molecule when it is added to the cell or system exogenously.

Various host (animals or cell systems) can be used to produce the proteins described herein. Examples of suitable host cells include, but are not limited to, bacteria, fungi (including yeasts), plant, insect, mammal, or other appropriate animal cells or cell lines, as well as transgenic animals or plants. In some embodiments, these hosts may include well known eukaryotic and prokaryotic hosts, such as strains of E. coli, Pseudomonas, Bacillus, Streptomyces, fungi, yeast, insect cells such as Spodoptera frugiperda (Sf9), animal cells such as Chinese hamster ovary (CHO) and mouse cells such as NS/O, African green monkey cells such as COS 1, COS 7, BSC 1, BSC 40, and BNT 10, and human cells, as well as plant cells in tissue culture. For animal cell expression, CHO cells and COS 7 cells in cultures and particularly the CHO cell line CHO (DHFR-) or the HKB line may be used. The proteins can also be expressed in vivo by delivering a nucleic acid molecule to a cell in vivo and having the cell express the protein, whereby the polypeptide as provided will traffic to the surface of the cell. In some embodiments, the nucleic acid molecule is delivered with a vector, such a viral vector, including but not limited to adenoviral vectors. In some embodiments, the nucleic acid molecule is encapsulated in a nanoparticle, such as a lipid nanoparticle to deliver the nucleic acid molecule to the cell. In some embodiments, the encapsulated nucleic acid molecule is a RNA molecule. In some embodiments, the encapsulated nucleic acid molecule is a DNA molecule.

One of skill in the art may make a selection among various vectors, expression control sequences and hosts without undue experimentation. For example, in selecting a vector, a host can be considered because the vector must be able replicate in it or the polypeptide must be able to be transcribed and/or translated from the vector in the specific host. The vectors copy number, the ability to control that copy number, and the expression of any other proteins encoded by the vector, such as antibiotic markers, can also be considered. Such amplifiable vectors are well known in the art.

Variants of the molecules (e.g., nucleic acid molecules and polypeptides) are provided for herein based on either percent identity or percent homology. The variants can encompass mutations, such as substitutions, insertions, or deletions, that modify the primary structure (sequence) of such molecules without impacting the activity of the polypeptide that is produced or used. For example, the variants of the polypeptides provided for herein can have 1, 2, 3, 4, 5o 6, 7, 8, 9, or 10 substitutions as compared to the reference sequence. These can be point mutations (substitutions), insertions or deletions. For clarity, an insertion or deletion of two or more contiguous amino acid residues is considered a single mutation, but an insertion or deletion of two different amino acid residues that are not contiguous are considered separate mutations.

For example, a variant of a IL-12 p40 polypeptide will function, in conjunction with IL-12 p35 as IL-12 and have the activity proscribed for IL-12. In some embodiments, a variant of a IL-12 p35 polypeptide will function, in conjunction with IL-12 p40 as IL-12 and have the activity proscribed for IL-12. In some embodiments, the variant IL-15 polypeptide has the IL-15 activity and can bind to the IL-15Rα polypeptide. In some embodiments, the variant IL-15Rα polypeptide has the IL-15Rα activity and can bind to the IL-15 polypeptide.

### Vectors and Host Cells

Accordingly, in some embodiments, vectors encoding the polypeptides described herein are provided, as well as host cells transformed or transduced with such vectors. Any nucleic acids encoding the proteins described herein may be contained in a vector, which can, for example, comprise a selectable marker and an origin of replication, for propagation in a host. In some embodiments, the vectors further include suitable transcriptional or translational regulatory sequences, such as those derived from a mammalian, microbial, viral, or insect genes, operably linked to the nucleic acid molecule encoding the protein. Examples of such regulatory sequences include transcriptional promoters, operators, or enhancers, mRNA ribosomal binding sites, and appropriate sequences that control transcription and translation. Nucleotide sequences are operably linked when the regulatory sequence functionally relates to the DNA encoding the target protein. Thus, a promoter nucleotide sequence is operably linked to a nucleic acid molecule if the promoter nucleotide sequence directs the transcription of the nucleic acid molecule.

The host cells that can be used here are described herein.

In some embodiments, a vector is provided comprising a nucleic acid molecule as provided for herein. In some embodiments, the vector is a plasmid. In some embodiments, the vector is an encapsulated vector, such as provided for herein. In some embodiments, the vector is a viral vector, such as, but not limited to an adenovirus or an adeno-associated virus, or a lentivirus. In some embodiments, the adenovirus is replication-incompetent. In some embodiments, the adenovirus is replication-competent.

In some embodiments, a cell is provided that comprises a polypeptide as provided for herein. In some embodiments, a cell is provided that comprises a genomic nucleic acid molecule comprising the nucleic acid molecule as provided for herein. A genomic nucleic acid molecule refers to a heterologous nucleic acid molecule that is integrated into the genome of the host cell.

In some embodiments, the cell further comprises a chimeric antigen receptor. Chimeric antigen receptors, or “CAR”, can be used to treat cancer or tumors in a subject. In some embodiments, the activity of the CAR can be enhanced by co-expressing a polypeptide as provided for herein with the CAR. Thus, in some embodiments, a cell is provided comprising a CAR and a polypeptide as provided for herein.

The cell can be any type of suitable cell. In some embodiments, the cell is an immune cell, such as, but not limited to a T-cell, a NK cell, a dendritic cell, and the like.

The cell can be produced according to any known methods. For example, in some embodiments, the method of producing a cell that is provided comprises contacting the cell with a vector comprising a nucleic acid molecule encoding for a polypeptide as provided for herein. This can be done, for example, under conditions that are suitable to express the polypeptide in the cell and to express on the surface of the cell. In some embodiments, the vector that is used is a plasmid or a virus. In some embodiments, the virus is an adenovirus, AAV, or lentivirus. In some embodiments, the adenovirus is a replication-incompetent or replication-competent adenovirus. In some embodiments, the contacting comprises transducing or transfecting the cell with the vector. In some embodiments, the vector further comprises a nucleic acid molecule encoding for a chimeric antigen receptor or a tumor antigen.

In some embodiments, methods of producing a cell comprising a polypeptide as provided herein in vivo are provided, the methods comprising administering to a subject a vector encoding for the polypeptide to a subject, wherein the vector transduces or transfects a cell in vivo to produce the cell comprising a polypeptide as provided for herein. In some embodiments, the cell is an immune cell. In some embodiments, the immune cell is, but not limited to a T cell, a NK cell, a dendritic cell, and the like. In some embodiments, the vector further comprises a nucleic acid molecule encoding for a chimeric antigen receptor or a tumor antigen. In some embodiments, the vector is a plasmid or a virus. In some embodiments, the virus is an adenovirus, AAV, or lentivirus. In some embodiments, the adenovirus is a replication-incompetent or replication-competent adenovirus.

### Pharmaceutical Compositions

In another aspect, the present embodiments provide compositions, e.g., pharmaceutically acceptable compositions, which include a polypeptide as provided for herein or a nucleic acid molecule encoding the same, which can be, for example, be formulated together with one or more excipients. In some embodiments, suitable excipients include, but are not limited to purified water, calcium carbonate, calcium phosphate, various sugars, starches, cellulose derivatives, gelatin, polymers such as polyethylene glycols, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof.

In some embodiments, a pharmaceutical composition is provided that comprises a cell comprising a vector, polypeptide, or nucleic acid molecule as provided for herein.

In some embodiments, the pharmaceutical composition further comprises a pharmaceutically acceptable carrier. In some embodiments, suitable pharmaceutically acceptable carries include, but are not limited to, water, silicone, waxes, petroleum jelly, polyethylene glycol, propylene glycol, liposomes, a lipid such as cholesterol, cationic lipids such as 1,2,-dioleoyl-3-trimethylammonium propane (DOTAP), 1,2,-dioleoyl-sn-glycero-3-phosphochiline (DOPC), and 1,2-dioleoyl-sn-glycero-3-phosphoethanolamine (DOPE), sugars such as mannitol and lactose, and other materials depending on the specific type of formulation used. In some embodiments, suitable pharmaceutically acceptable carries include, but are not limited to, nanoparticles such as gold or metallic nanoparticles.

In some embodiments, the lipids and liposomes comprises a cationic lipid, such as, but not limited to, 1,2-Dioleoyl-3-Trimethylammonium-Propane (DOTAP), 1,2,-dioleoyl-sn-glycero-3-phosphochiline (DOPC), 1,2-dioleoyl-sn-glycero-3-phosphoethanolamine (DOPE), N-[1-(2,3-dioleyloxy)propyl]-N,N,N-trimethylammonium chloride (DOTMA), 5-carboxyspermylglycinedioctadecylamide (DOGS), 2,3-dioleyloxy-N-[2(spermine-carboxamido)ethyl]-N,N-dimethyl-1-propanamin-ium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraenl9-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis,cis-9′,1- -2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), or C12-200.

In some embodiments, the pharmaceutically acceptable carrier can be suitable for intravenous, intramuscular, subcutaneous, parenteral, rectal, local, topical, spinal or epidermal administration (e.g. by injection or infusion). In some embodiments, the pharmaceutical composition comprises a vector comprising a nucleic acid molecule encoding a polypeptide as provided for herein. In some embodiments, the nucleic acid molecule is a DNA molecule or a RNA molecule. In some embodiments, the vector is a virus, such as those provided for herein.

The compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments the mode of administration is parenteral (e.g., intravenous, subcutaneous, intraperitoneal, intradermal, intramuscular, intravesicular). In some embodiments, the composition is administered by intravenous infusion or injection. In some embodiments, the composition is administered by intramuscular or subcutaneous injection. In some embodiments, the composition is administered by enteral, sublingual, inhalation, or intranasal. In some embodiments, the composition is administered locally, e.g., by injection, or topical application, to a target site. For example, the pharmaceutical compositions can be lyophilized and reconstituted for use prior to administration to the patient.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Compositions, such as pharmaceutical compositions, typically are sterile and stable under the conditions of manufacture and storage. The composition can be formulated as a solution, microemulsion, dispersion, liposome, or other ordered structure suitable for a high concentration of the active ingredient. Sterile injectable solutions can be prepared by incorporating the active compound (i.e., therapeutic molecule, nucleic acid molecule, cell, polypeptide, vector, etc.) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the pharmaceutical composition can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compositions as provided for herein by other than parenteral administration, it may be necessary to coat the compositions with, or co-administer the compositions with, a material to prevent its inactivation. The compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks, or, in some embodiments, the dosing schedule can be, once every month, every 2 months, every 3 months, or every 6 months. In one embodiment, the therapeutic compound is administered at a dose from about 10 to 20 mg/kg every other week.

The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of the compositions, vectors, cells, polypeptides, or nucleic acid molecules encoding the same. A “therapeutically effective amount” refers to an amount effective, at dosages and for periods of time necessary, to achieve the desired therapeutic result. A therapeutically effective amount of an active ingredient or molecule may vary according to factors such as the disease state, age, sex, and weight of the individual, and the ability of the therapeutic compound to elicit a desired response in the individual. A “therapeutically effective dosage” can, for example, inhibit a measurable parameter, e.g., tumor growth, by at least about 20%, by at least about 40%, by at least about 60%, and by at least about 80% relative to untreated subjects. The ability of a compound to inhibit a measurable parameter, e.g., tumor growth, can be evaluated in an animal model system predictive of efficacy in tumor growth. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount can be, but is not necessarily, less than the therapeutically effective amount.

Also provided herein are kits comprising compositions, cells, vectors, nucleic acid molecules, or polypeptides as described herein. The kit can include one or more other elements including: instructions for use; other reagents, e.g., a label, a therapeutic agent, or an agent useful for chelating, or otherwise coupling, a molecule to a label or other therapeutic agent, or a radioprotective composition; devices or other materials for preparing the molecule for administration; pharmaceutically acceptable carriers; and devices or other materials for administration to a subject.

### Combinations

The compositions provided herein can also be administered in conjunction with other agents useful for treating the condition with which the patient is suffering from. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in compositions provided for herein are administered at least once during a course of treatment that involves administering at least one other therapeutic agent to the patient.

In some embodiments, the compositions provided herein can be combined, linked, or fused to at least one additional molecule. In some embodiments, the additional molecule is a biologically active molecule, for example, but not limited to, a protein, a polypeptide, a nucleic acid, a lipid, carbohydrate, or any combination thereof. In some embodiments, the additional molecule is a targeting moiety, for example, but not limited to, an antibody, an antigen, a ligand, a ligand trap such as a receptor domain, or any combination thereof. In some embodiments, the additional molecule is a therapeutic molecule, for example, but not limited to, an immunotherapeutic molecule, a checkpoint inhibitor, an immune system activator, an oncological therapeutic, an antibody, or any combination thereof.

In some embodiments, the compositions provided herein can be attached to the at least one additional molecule at the C-terminus of the composition. In some embodiments, the compositions provided herein can be attached to the at least one additional molecule at the N-terminus of the composition. In some embodiments, the compositions provided herein can be attached to the at least one additional molecule at any linker of the composition. In some embodiments, the compositions provided herein can be attached to the at least one additional molecule before any cleavable linkers have been cleaved. In some embodiments, the compositions provided herein can be attached to the at least one additional molecule after the composition has been cleaved at a cleavable linker.

### Therapeutic Methods

“Treatment” of any disease mentioned herein encompasses an alleviation of at least one symptom of the disease, a reduction in the severity of the disease, or the delay or prevention of disease progression to more serious symptoms that may, in some cases, accompany the disease or to at least one other disease. Treatment need not mean that the disease is totally cured. A useful therapeutic agent needs only to reduce the severity of a disease, reduce the severity of symptom(s) associated with the disease or its treatment, or delay the onset of more serious symptoms or a more serious disease that can occur with some frequency following the treated condition. For example, if the disease is a tumor, the composition may reduce the growth or spread of the tumor, or the tumors effect on the tissue in which it is present. A patient's condition can be assessed by standard techniques. Suitable procedures vary according to the patient's condition and symptoms.

In some embodiments, the compositions provided for herein can be used to modify an immune response in a patient. In some embodiments, the methods comprise administering to the patient a polypeptide or a vector comprising a nucleic acid molecule encoding for a polypeptide as provided for herein. In some embodiments, the immune response is an activated immune response, such as in activating NK and/or CD8+ T cells.

In some embodiments, the compositions provided for herein can be used to treat cancer in a subject (patient). In some embodiments, the methods comprise administering to the patient a vector comprising a nucleic acid molecule encoding for a polypeptide as provided for herein. In some embodiments, the cancer is lymphoma, leukemia, nasopharyngeal, gastric, cervical, hepatocellular, polyoma, anal, head and neck tumor. In some embodiments, the tumor is a lung cancer tumor. In some embodiments, the tumor is benign and metastatic forms of cancer, for example, ovarian cancer (e.g. ovarian carcinoma), reproductive cancers (breast, cervical, testicular, uterine, and placental cancers), lung cancer, gastric cancer, hepatic cancer, pancreatic cancer, bile duct cancer, cancer of the urinary bladder, kidney cancer, colon cancer, small bowel cancer, skin cancer, brain cancer, head and neck cancer, sarcoma, and germ cell tumors, among others.

In some embodiments, diseases that can be treated with the compositions provided for herein also include myelodysplastic syndrome (MDS) or acute myeloid leukemia (AML). In some embodiments, the subject has MDS including Fanconi Anemia, refractory anemia, refractory neutropenia, refractory thrombocytopenia, refractory anemia with ringed sideroblasts (RARS), refractory cytopenia with multilineage dysplasia (RCMD), refractory anemia with multilineage dysplasia and ringed sideroblasts (RCMD-RS), refractory anemia with excess blasts I and II (RAEB), myelodysplastic syndrome, unclassified (MDS-U), MDS associated with isolated del(5q)-syndrome, chronic myelomonocytic leukemia (CMML), juvenile myelomonocytic leukemia (JMML), refractory cytopenia of childhood, or a combination thereof. In some embodiments, the subject has AML including AML with recurrent genetic abnormalities (AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3), AML (megakaryoblastic) with a translocation between chromosomes 1 and 22, AML with myelodysplasia-related changes, AML related to previous chemotherapy or radiation (alkylating agent-related AML, topoisomerase II inhibitor-related AML), AML not otherwise categorized (AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis), myeloid sarcoma (also known as granulocytic sarcoma, chloroma or extramedullary myeloblastoma), undifferentiated and biphenotypic acute leukemias (also known as mixed phenotype acute leukemias), or a combination thereof. In some embodiments, administration of the compositions provided for herein to a subject decreases the incidence of one or more symptoms associated with MDS or AML or decreases one or more markers of viability of MDS or AML cells. In some embodiments, the one or more symptoms associated with MDS or AML include decreasing marrow failure, immune dysfunction, transformation to overt leukemia, or a combination thereof in the subject, or wherein the marker of viability of MDS or AML cells includes survival over time, proliferation, growth, migration, formation of colonies, chromatic assembly, DNA binding, RNA metabolism, cell migration, cell adhesion, inflammation, or a combination thereof.

In some embodiments, the tumor is also treated with a checkpoint inhibitor. In some embodiments, the tumor is also treated with a PD-1 inhibitor, such as a PD-1 antagonist, such as PD-1 antagonist antibodies. In some embodiments, the tumor is also treated with a PD-L1 inhibitor, such as a PD-L1 antagonist, such as PD-L1 antagonist antibodies. In some embodiments, the tumor is also treated with a CTLA-4 inhibitor, such as a CTLA-4 antagonist, such as CTLA-4 antagonist antibodies.

In some embodiments, the compositions provided for herein can be used to treat a viral infection, a bacterial infection, or a fungal infection in a subject (patient).

In some embodiments, the methods comprise administering a pharmaceutical composition comprising the polypeptides provided herein or a nucleic acid molecule encoding the same as provided for herein to the subject. In some embodiments, the subject is a subject in need thereof. Any of the above-described can be administered in the form of a compositions (e.g. pharmaceutical compositions) that are described herein.

To treat the disease of interest, the compositions comprising the cells, vectors, nucleic acid molecules, or polypeptides described herein can be administered by any appropriate method including, but not limited to, parenteral, topical, oral, nasal, vaginal, rectal, or pulmonary (by inhalation) administration. If injected, the composition(s) can be administered intra-articularly, intravenously, intraarterially, intramuscularly, intravesicularly, intraperitoneally, or subcutaneously by bolus injection or continuous infusion. Localized administration, that is, at the site of disease, is contemplated, as are transdermal delivery and sustained release from implants, skin patches, or suppositories. Delivery by inhalation includes, for example, nasal or oral inhalation, use of a nebulizer, inhalation in aerosol form, and the like. Administration via a suppository inserted into a body cavity can be accomplished, for example, by inserting a solid form of the composition in a chosen body cavity and allowing it to dissolve. Other alternatives include eyedrops, oral preparations such as pills, lozenges, syrups, and chewing gum, and topical preparations such as lotions, gels, sprays, and ointments.

In the performance of the methods of treatment, the compositions described herein can be administered as described herein and above. For example, the composition can be administered at any dosage, frequency, and duration that can be effective to treat the condition being treated. The dosage depends on the molecular nature of the active ingredient and the nature of the disorder being treated. Treatment may be continued as long as necessary to achieve the desired results. The compositions provided for herein can be administered as a single dosage or as a series of dosages given periodically, including multiple times per day, daily, every other day, twice a week, three times per week, weekly, every other week, and monthly dosages, among other possible dosage regimens. The periodicity of treatment may or may not be constant throughout the duration of the treatment. For example, treatment may initially occur at weekly intervals and later occur every other week. Treatments having durations of days, weeks, months, or years are encompassed by the embodiments provided for herein. Treatment may be discontinued and then restarted. Maintenance doses may or may not be administered after an initial treatment.

Dosage may be measured as milligrams per kilogram of body weight (mg/kg) or as milligrams per square meter of skin surface (mg/m²) or as a fixed dose, irrespective of height or weight. All of these are standard dosage units in the art. A person's skin surface area is calculated from her height and weight using a standard formula.

Also provided herein are methods of activating CD8+ T cells. In some embodiments, methods provided herein can be used to activate NK cell. In some embodiments, the methods comprise administering to a subject in need thereof a therapeutically effective amount of a polypeptide or a nucleic acid molecule encoding the same, or vector comprising the same or as otherwise described herein or a pharmaceutical composition comprising the same.

As used herein, the phrase “in need thereof” means that the subject (animal or mammal) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

### Embodiments

In some embodiments, embodiments provided herein also include, but are not limited to:

1. A polypeptide comprising the formula of: X₁-L₁-X₂-L₂-X₃-L₃-X₄, wherein:
X₁ is a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Ra) polypeptide;
X₂ is a IL-15 polypeptide, IL-12 p40 polypeptide, IL-12 p35 polypeptide, or IL-15Ra polypeptide;
X₃ is a IL-15 polypeptide, IL-12 p40 polypeptide, IL-12 p35 polypeptide, or IL-15Ra polypeptide;
X₄ is a IL-15 polypeptide, IL-12 p40 polypeptide, IL-12 p35 polypeptide, or IL-15Ra polypeptide;
L₁, L₂, and L₃ are each, independently, a polypeptide linker that comprise the same or different polypeptide sequences,
provided each of X₁, X₂, X₃, and X₄ are different.
   2. The polypeptide of embodiment 1, wherein:
X₁ is a IL-12 p40 polypeptide;
X₂ is a IL-12 p35 polypeptide;
X₃ is a IL-15 polypeptide; and
X₄ is a IL-15Ra polypeptide.
   3. The polypeptide of embodiment 1, wherein:
X₁ is a IL-12 p35 polypeptide;
X₂ is a IL-12 p40 polypeptide;
X₃ is a IL-15 polypeptide; and
X₄ is a IL-15Ra polypeptide.
   4. The polypeptide of embodiment 1, wherein:
X₁ is a IL-15 polypeptide;
X₂ is a IL-12 p40 polypeptide;
X₃ is a IL-12 p35 polypeptide; and
X₄ is a IL-15Ra polypeptide.
   5. The polypeptide of embodiment 1, wherein:
X₁ is a IL-15 polypeptide;
X₂ is a IL-12 p35 polypeptide;
X₃ is a IL-12 p40 polypeptide; and
X₄ is a IL-15Ra polypeptide.
   6. The polypeptide of embodiment 1, wherein:
X₁ is a IL-12 p35 polypeptide;
X₂ is a IL-15 polypeptide;
X₃ is a IL-12 p40 polypeptide; and
X₄ is a IL-15Ra polypeptide.
   7. The polypeptide of embodiment 1, wherein:
X₁ is a IL-12 p40 polypeptide;
X₂ is a IL-15 polypeptide;
X₃ is a IL-12 p35 polypeptide; and
X₄ is a IL-15Ra polypeptide.
   8. A polypeptide comprising the formula of: X₁-L1-X₂-L₂-X₃-L₃-X₄-L₄-X₅, wherein:
X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Ra) polypeptide, a first fragment of IL-15Ra polypeptide, or a second fragment of IL-15Ra polypeptide;
X₂ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₃ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₄ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₅ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
L₁, L₂, L₃, and L₄ are each, independently, a polypeptide linker that comprise the same or different polypeptide sequences,
provided each of X₁, X₂, X₃, X₄, and X₅ are different.
   9. The polypeptide of embodiment 8, wherein:
X₁ is a IL-15 polypeptide;
X₂ is a first fragment of IL-15Ra polypeptide;
X₃ is a IL-12 p40 polypeptide;
X₄ is a IL-12 p35 polypeptide; and
X₅ is a second fragment of IL-15Ra polypeptide.
   10. The polypeptide of embodiment 8, wherein:
X₁ is a IL-15 polypeptide;
X₂ is a first fragment of IL-15Ra polypeptide;
X₃ is a IL-12 p35 polypeptide;
X₄ is a IL-12 p40 polypeptide; and
X₅ is a second fragment of IL-15Ra polypeptide.
   11. The polypeptide of embodiment 8, wherein:
X₁ is a IL-15 polypeptide;
X₂ is a IL-12 p40 polypeptide;
X₃ is a first fragment of IL-15Ra polypeptide;
X₄ is a IL-12 p35 polypeptide; and
X₅ is a second fragment of IL-15Ra polypeptide.
   12. The polypeptide of embodiment 8, wherein:
X₁ is a IL-15 polypeptide;
X₂ is a IL-12 p40 polypeptide;
X₃ is a IL-12 p35 polypeptide;
X₄ is a first fragment of IL-15Ra polypeptide; and
X₅ is a second fragment of IL-15Ra polypeptide.
   13 The polypeptide of embodiment 8, wherein:
X₁ is a IL-15 polypeptide;
X₂ is a IL-12 p35 polypeptide;
X₃ is a IL-12 p40 polypeptide;
X₄ is a first fragment of IL-15Ra polypeptide; and
X₅ is a second fragment of IL-15Ra polypeptide.
   14. The polypeptide of embodiment 8, wherein:
X₁ is a IL-15 polypeptide;
X₂ is a IL-12 p35 polypeptide;
X₃ is a first fragment of IL-15Ra polypeptide;
X₄ is a IL-12 p40 polypeptide; and
X₅ is a second fragment of IL-15Ra polypeptide.
   15. The polypeptide of embodiment 8, wherein:
X₁ is a first fragment of IL-15Ra polypeptide;
X₂ is a IL-15 polypeptide;
X₃ is a IL-12 p40 polypeptide;
X₄ is a IL-12 p35 polypeptide; and
X₅ is a second fragment of IL-15Ra polypeptide.
   16. The polypeptide of embodiment 8, wherein:
X₁ is a first fragment of IL-15Ra polypeptide;
X₂ is a IL-15 polypeptide;
X₃ is a IL-12 p35 polypeptide;
X₄ is a IL-12 p40 polypeptide; and
X₅ is a second fragment of IL-15Ra polypeptide.
   17. The polypeptide of embodiment 8, wherein:
X₁ is a first fragment of IL-15Ra polypeptide;
X₂ is a IL-12 p40 polypeptide;
X₃ is a IL-15 polypeptide;
X₄ is a IL-12 p35 polypeptide; and
X₅ is a second fragment of IL-15Ra polypeptide.
   18. The polypeptide of embodiment 8, wherein:
X₁ is a first fragment of IL-15Ra polypeptide;
X₂ is a IL-12 p40 polypeptide;
X₃ is a IL-12 p35 polypeptide;
X₄ is a IL-15; and
X₅ is a second fragment of IL-15Ra polypeptide.
   19. The polypeptide of embodiment 8, wherein:
X₁ is a first fragment of IL-15Ra polypeptide;
X₂ is a IL-12 p35 polypeptide;
X₃ is a IL-12 p40 polypeptide;
X₄ is a IL-15; and
X₅ is a second fragment of IL-15Ra polypeptide.
   20. The polypeptide of embodiment 8, wherein:
X₁ is a first fragment of IL-15Ra polypeptide;
X₂ is a IL-12 p35 polypeptide;
X₃ is a IL-15;
X₄ is a IL-12 p40 polypeptide; and
X₅ is a second fragment of IL-15Ra polypeptide.
   21. The polypeptide of embodiment 8, wherein:
X₁ is a IL-12 p40 polypeptide;
X₂ is a IL-15 polypeptide;
X₃ is a first fragment of IL-15Ra polypeptide;
X₄ is a IL-12 p35 polypeptide; and
X₅ is a second fragment of IL-15Ra polypeptide.
   22. The polypeptide of embodiment 8, wherein:
X₁ is a IL-12 p40 polypeptide;
X₂ is a IL-15 polypeptide;
X₃ is a IL-12 p35 polypeptide;
X₄ is a first fragment of IL-15Ra polypeptide; and
X₅ is a second fragment of IL-15Ra polypeptide.
   23. The polypeptide of embodiment 8, wherein:
X₁ is a IL-12 p40 polypeptide;
X₂ is a first fragment of IL-15Ra polypeptide;
X₃ is a IL-15 polypeptide;
X₄ is a IL-12 p35 polypeptide; and
X₅ is a second fragment of IL-15Ra polypeptide.
   24. The polypeptide of embodiment 8, wherein:
X₁ is a IL-12 p40 polypeptide;
X₂ is a first fragment of IL-15Ra polypeptide;
X₃ is a IL-12 p35 polypeptide;
X₄ is a IL-15; and
X₅ is a second fragment of IL-15Ra polypeptide.
   25. The polypeptide of embodiment 8, wherein:
X₁ is a IL-12 p40 polypeptide;
X₂ is a IL-12 p35 polypeptide;
X₃ is a first fragment of IL-15Ra polypeptide;
X₄ is a IL-15; and
X₅ is a second fragment of IL-15Ra polypeptide.
   26. The polypeptide of embodiment 8, wherein:
X₁ is a IL-12 p40 polypeptide;
X₂ is a IL-12 p35 polypeptide;
X₃ is a IL-15;
X₄ is a first fragment of IL-15Ra polypeptide; and
X₅ is a second fragment of IL-15Ra polypeptide.
   27. The polypeptide of embodiment 8, wherein:
X₁ is a IL-12 p35 polypeptide;
X₂ is a IL-15 polypeptide;
X₃ is a first fragment of IL-15Ra polypeptide;
X₄ is a IL-12 p40 polypeptide; and
X₅ is a second fragment of IL-15Ra polypeptide.
   28. The polypeptide of embodiment 8, wherein:
X₁ is a IL-12 p35 polypeptide;
X₂ is a IL-15 polypeptide;
X₃ is a IL-12 p40 polypeptide;
X₄ is a first fragment of IL-15Ra polypeptide; and
X₅ is a second fragment of IL-15Ra polypeptide.
   29. The polypeptide of embodiment 8, wherein:
X₁ is a IL-12 p35 polypeptide;
X₂ is a first fragment of IL-15Ra polypeptide;
X₃ is a IL-15 polypeptide;
X₄ is a IL-12 p40 polypeptide; and
X₅ is a second fragment of IL-15Ra polypeptide.
   30. The polypeptide of embodiment 8, wherein:
X₁ is a IL-12 p35 polypeptide;
X₂ is a first fragment of IL-15Ra polypeptide;
X₃ is a IL-12 p40 polypeptide;
X₄ is a IL-15; and
X₅ is a second fragment of IL-15Ra polypeptide.
   31. The polypeptide of embodiment 8, wherein:
X₁ is a IL-12 p35 polypeptide;
X₂ is a IL-12 p40 polypeptide;
X₃ is a first fragment of IL-15Ra polypeptide;
X₄ is a IL-15; and
X₅ is a second fragment of IL-15Ra polypeptide.
   32. The polypeptide of embodiment 8, wherein:
X₁ is a IL-12 p35 polypeptide;
X₂ is a IL-12 p40 polypeptide;
X₃ is a IL-15;
X₄ is a first fragment of IL-15Ra polypeptide; and
X₅ is a second fragment of IL-15Ra polypeptide.
   33. A polypeptide comprising the formula of: X₁-L₁-X₂, wherein:
X₁ is a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Ra) polypeptide;
X₂ is a IL-15 polypeptide, IL-12 p40 polypeptide, IL-12 p35 polypeptide, or IL-15Ra polypeptide;
L₁ is a polypeptide linker,
provided X₁ and X₂ are different.
   34. The polypeptide of embodiment 33, wherein:
X₁ is a IL-15 polypeptide; and
X₂ is a IL-15Ra polypeptide.
   35. A polypeptide comprising the formula of: X₁-L₁-X₂-L₂-X₃, wherein:
X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Ra) polypeptide, a first fragment of IL-15Ra polypeptide, or a second fragment of IL-15Ra polypeptide;
X₂ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₃ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
L₁ and L₂ are each, independently, a polypeptide linker that comprise the same or different polypeptide sequences,
provided each of X₁, X₂, and X₃ are different.
   36. The polypeptide of embodiment 35, wherein:
X₁ is a IL-12 p40 polypeptide;
X₂ is a IL-12 p35 polypeptide; and
X₃ is a second fragment of IL-15Ra polypeptide.
   37. The polypeptide of embodiment 35, wherein:
X₁ is a IL-12 p35 polypeptide;
X₂ is a IL-12 p40 polypeptide; and
X₃ is a second fragment of IL-15Ra polypeptide.
   38. The polypeptide of embodiment 35, wherein:
X₁ is a IL-15 polypeptide;
X₂ is a IL-12 p35 polypeptide; and
X₃ is a second fragment of IL-15Ra polypeptide.
   39. The polypeptide of embodiment 35, wherein:
X₁ is a IL-15 polypeptide;
X₂ is a IL-12 p40 polypeptide; and
X₃ is a second fragment of IL-15Ra polypeptide.
   40. The polypeptide of any one of embodiments 1-39, wherein the IL-12 p40 polypeptide comprises an amino acid sequence having at least 90%, 91%, 92%, 93%, 94%, 95%, 96%, 97%, 98%, or 99% identity to SEQ ID NO: 1, 2, 3, or 4, or as otherwise provided for herein.
   41. The polypeptide of any one of embodiments 1-40, wherein the IL-12 p40 polypeptide comprises an amino acid sequence of SEQ ID NO: 1, 2, 3, or 4, or as otherwise provided for herein.
   42. The polypeptide of any one of embodiments 1-41, wherein the IL-12 p40 polypeptide comprises a IL-15 leader sequence at the N-terminus of the polypeptide.
   43. The polypeptide of embodiment 42, wherein the IL-15 leader sequence comprises an amino acid sequence having at least 90%, 91%, 92%, 93%, 94%, 95%, 96%, 97%, 98%, or 99% identity to SEQ ID NO: 15 or as otherwise provided for herein.
   44. The polypeptide of embodiments 42 or 43, wherein the IL-15 leader sequence comprises an amino acid sequence of SEQ ID NO: 15 or as otherwise provided for herein.
   45. The polypeptide of any one of embodiments 1-44, wherein the IL-12 p35 polypeptide comprises an amino acid sequence having at least 90%, 91%, 92%, 93%, 94%, 95%, 96%, 97%, 98%, or 99% identity to SEQ ID NO: 5, 6, 7, or 8, or as otherwise provided for herein.
   46. The polypeptide of any one of embodiments 1-45, wherein the IL-12 p35 polypeptide comprises an amino acid sequence of SEQ ID NO: 5, 6, 7, or 8, or as otherwise provided for herein.
   47. The polypeptide of any one of embodiments 1-46, wherein the IL-12 p35 polypeptide comprises a IL-15 leader sequence at the N-terminus of the polypeptide.
   48. The polypeptide of embodiment 47, wherein the IL-15 leader sequence comprises an amino acid sequence having at least 90%, 91%, 92%, 93%, 94%, 95%, 96%, 97%, 98%, or 99% identity to SEQ ID NO: 15 or as otherwise provided for herein.
   49. The polypeptide of embodiments 47 or 48, wherein the IL-15 leader sequence comprises an amino acid sequence of SEQ ID NO: 15 or as otherwise provided for herein.
   50. The polypeptide of any one of embodiments 1-49, wherein the IL-15 polypeptide comprises an amino acid sequence having at least 90%, 91%, 92%, 93%, 94%, 95%, 96%, 97%, 98%, or 99% identity to SEQ ID NO: 9 or 10 or as otherwise provided for herein.
   51. The polypeptide of any one of embodiments 1-50, wherein the IL-15 polypeptide comprises an amino acid sequence of SEQ ID NO: 9 or ‘0 or as otherwise provided for herein.
   52. The polypeptide of any one of embodiments 1-51, wherein the IL-15Ra polypeptide comprises an amino acid sequence having at least 90%, 91%, 92%, 93%, 94%, 95%, 96%, 97%, 98%, or 99% identity to SEQ ID NO: 11 or 12 or as otherwise provided for herein.
   53. The polypeptide of any one of embodiments 1-52, wherein the IL-15Ra polypeptide comprises an amino acid sequence of SEQ ID NO: 11 or 12 or as otherwise provided for herein.
   54. The polypeptide of any one of embodiments 1-53 wherein the first fragment of IL-15Ra polypeptide comprises an amino acid sequence having at least 90%, 91%, 92%, 93%, 94%, 95%, 96%, 97%, 98%, or 99% identity to SEQ ID NO: 13 or as otherwise provided for herein.
   55. The polypeptide of any one of embodiments 1-54, wherein the first fragment of IL-15Ra polypeptide comprises an amino acid sequence of SEQ ID NO: 13 or as otherwise provided for herein.
   56. The polypeptide of any one of embodiments 1-55, wherein the second fragment of IL-15Ra polypeptide comprises an amino acid sequence having at least 90%, 91%, 92%, 93%, 94%, 95%, 96%, 97%, 98%, or 99% identity to SEQ ID NO: 14 or as otherwise provided for herein.
   57. The polypeptide of any one of embodiments 1-52, wherein the second fragment of IL-15Ra polypeptide comprises an amino acid sequence of SEQ ID NO: 14 or as otherwise provided for herein.
   58. The polypeptide of any one of embodiments 1-575, wherein one or more of L₁, L₂, L₃, and L₄ are each, independently, a cleavable linker.
   59. The polypeptide of embodiment 58, wherein the cleavable linker is a furin cleavable linker, a Val-Cit linker, a Val-Gly linker, a Gly-Gly linker, an Ala-Ala-Asn linker, or a linker comprising polyethylene glycol (PEG).
   60. The polypeptide of any one of embodiments 1-59, wherein one of L₁, L₂, L₃, and L₄ comprise a sequence of (GSGSGG)ₙ (SEQ ID NO: 16), (GGGGS)ₙ (SEQ ID NO: 17), (GGGGA)ₙ (SEQ ID NO: 18), (GGGSE)ₙ (SEQ ID NO: 19), (GGGSK)ₙ (SEQ ID NO: 20), or (AEEEK)ₙ (SEQ ID NO: 21), wherein each n is, independently, 1, 2, 3, 4, or 5, or a combination thereof.
   61. The polypeptide of embodiment 60, wherein n is 3.
   62. The polypeptide of embodiments 60 or 61, wherein one or more of L₁, L₂, L₃, and L₄ comprise a sequence of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 69, SEQ ID NO: 70, or a combination thereof.
   63. The polypeptide of any one of embodiments 60-62, wherein each of L₁, L₂, L₃, and L₄ comprises a sequence of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 69, SEQ ID NO: 70, or a combination thereof.
   64. The polypeptide of any one of embodiments 1-63, further comprising a leader peptide on the N-terminus of the polypeptide.
   65. The polypeptide of embodiment 64, wherein the leader peptide comprises a sequence of vesicular stomatitis virus G protein (VSV-G).
   66. The polypeptide of embodiment 64, wherein the leader peptide comprises, consists, or consists essentially of a sequence of MRISKPHLRSISIQCYLCLLLNSHFLTEA (SEQ ID NO: 15).
   67. The polypeptide of embodiment 64, wherein the leader peptide comprises a sequence of IL-15.
   68. The polypeptide of embodiment 64 or 67, wherein the leader peptide comprises an amino acid sequence having at least 90%, 91%, 92%, 93%, 94%, 95%, 96%, 97%, 98%, or 99% identity to SEQ ID NO: 15 or as otherwise provided for herein.
   69. The polypeptide of embodiments 64, 67 or 68, wherein the leader peptide comprises an amino acid sequence of SEQ ID NO: 15 or as otherwise provided for herein.
   70. The polypeptide of any one of embodiments 1-69, wherein the polypeptide comprises the sequence of SEQ ID NO: 51, a variant thereof, or as otherwise provided for herein.
   71. The polypeptide of any one of embodiments 1-69, wherein the polypeptide comprises the sequence of SEQ ID NO: 52, a variant thereof, or as otherwise provided for herein.
   72. The polypeptide of any one of embodiments 1-69, wherein the polypeptide comprises the sequence of SEQ ID NO: 53, a variant thereof, or as otherwise provided for herein.
   73. The polypeptide of any one of embodiments 1-69, wherein the polypeptide comprises the sequence of SEQ ID NO: 54, a variant thereof, or as otherwise provided for herein.
   74. The polypeptide of any one of embodiments 1-69, wherein the polypeptide comprises the sequence of SEQ ID NO: 55, a variant thereof, or as otherwise provided for herein.
   75. The polypeptide of any one of embodiments 1-69, wherein the polypeptide comprises the sequence of SEQ ID NO: 56, a variant thereof, or as otherwise provided for herein.
   76. The polypeptide of any one of embodiments 1-69, wherein the polypeptide comprises the sequence of SEQ ID NO: 57, a variant thereof, or as otherwise provided for herein.
   77. The polypeptide of any one of embodiments 1-69, wherein the polypeptide comprises the sequence of SEQ ID NO: 58, a variant thereof, or as otherwise provided for herein.
   78. The polypeptide of any one of embodiments 1-69, wherein the polypeptide comprises the sequence of SEQ ID NO: 59, a variant thereof, or as otherwise provided for herein.
   79. The polypeptide of any one of embodiments 1-69, wherein the polypeptide comprises the sequence of SEQ ID NO: 60, a variant thereof, or as otherwise provided for herein.
   80. The polypeptide of any one of embodiments 1-69, wherein the polypeptide comprises the sequence of SEQ ID NO: 61, a variant thereof, or as otherwise provided for herein.
   81. A pharmaceutical composition comprising the polypeptide of any one of embodiment 1-80 and a pharmaceutical excipient.
   82. The pharmaceutical composition of embodiment 81, wherein the pharmaceutical excipient is any pharmaceutical excipient described herein.
   83. The pharmaceutical composition of embodiments 80 or 81, further comprising a pharmaceutical carrier.
   84. The pharmaceutical composition of embodiment 83, wherein the pharmaceutical carrier is water.
   85. A nucleic acid (e.g. DNA, RNA, cRNA, or mRNA) molecule encoding a polypeptide any one of embodiments 1-80.
   86. The nucleic acid molecule of embodiment 85, wherein the nucleic acid molecule comprises of a nucleic acid sequence having at least 80%, 85%, 90%, 95%, 96%, 97%, 98%, 99% or 100% identity to SEQ ID NO: 62, SEQ ID NO: 63, SEQ ID NO: 64, SEQ ID NO: 65, SEQ ID NO: 66, or SEQ ID NO: 67.
   87. A vector comprising the nucleic acid molecule of embodiments 85 or 86.
   88. A plasmid comprising the nucleic acid molecule of embodiments 85 or 86.
   89. A recombinant virus comprising the nucleic acid molecule of embodiments 85 or 86.
   90. The recombinant virus of embodiment 89, wherein said virus is a recombinant adenovirus or lentivirus.
   91. The recombinant virus of embodiment 90, wherein said recombinant adenovirus is replication-incompetent or replication-competent.
   92. A cell comprising the polypeptide of any one of embodiments 1-80.
   93. A cell comprising a genomic nucleic acid molecule comprising the nucleic acid molecule of embodiments 85 or 86.
   94. The cell of embodiments 92 or 93, wherein the cell further comprises a chimeric antigen receptor (CAR).
   95. The cell of any one of embodiments 92-94, wherein the cell is an immune cell.
   96. The cell of embodiment 95, wherein the immune cell is a T-cell, a NK cell, or a dendritic cell.
   97. A method for producing the cell of any one of embodiments 92-96, the method comprising contacting the cell with the pharmaceutical composition of any one of embodiments 81-84, the vector of embodiment 87, the plasmid of embodiment 88, or a virus of any one of embodiments 89-91.
   98. The method of embodiment 97, wherein the contacting comprises transducing or transfecting the cell with the vector, the plasmid, or the virus.
   99. The method of embodiment 97 or 98, wherein the vector, the plasmid, or the virus further comprise a nucleic acid molecule encoding for a chimeric antigen receptor or a tumor antigen.
   100. A method of producing a cell comprising a polypeptide of any one of embodiments 1-80 in vivo, the method comprising administering to a subject the vector of embodiment 87, the plasmid of embodiment 88, or a virus of any one of embodiments 89-91, wherein the vector, plasmid, or virus transduces or transfects a cell in vivo to produce the cell comprising the polypeptide of any one of embodiments 1-80.
   101. The method of embodiment 100, wherein the cell is an immune cell.
   102. The method of embodiment 101, wherein the immune cell is, but not limited to a T-cell, a NK cell, a dendritic cell, and the like.
   103. The method of any one of embodiments 100-102, wherein the vector, plasmid, or virus further comprise a nucleic acid molecule encoding for a chimeric antigen receptor or a tumor antigen.
   104. A method for modifying an immune response in a patient, the method comprising administering to the patient the pharmaceutical composition of any one of embodiments 81-84, the vector of embodiment 87, the plasmid of embodiment 88, or a virus of any one of embodiments 89-91.
   105. The method of any one of embodiments 104, wherein the immune response is activated.
   106. The method of embodiment 104 or 105, wherein the vector, the plasmid, or the virus further comprise a nucleic acid molecule encoding for a chimeric antigen receptor and/or a tumor antigen.
   107. A method of treating a cancer in a subject, the method comprising administering to the subject the pharmaceutical composition of any one of embodiments 81-84, the vector of embodiment 87, the plasmid of embodiment 88, or a virus of any one of embodiments 89-91.
   108. The method of embodiment 107, wherein the cancer is as provided for herein.
   109. The method of embodiment 107, wherein the vector, the plasmid, or the virus further comprise a nucleic acid molecule encoding for a chimeric antigen receptor and/or a tumor antigen.
   110. A method of treating a disease or disorder, such as a viral infection, bacterial infection or a fungal infection, such as those provided herein, the method comprising administering to the subject the pharmaceutical composition of any one of embodiments 81-84, the vector of embodiment 87, the plasmid of embodiment 88, or a virus of any one of embodiments 89-91.

### EXAMPLES

The following examples are illustrative, but not limiting, of the compounds, compositions and methods described herein. Other suitable modifications and adaptations known to those skilled in the art are within the scope of the following embodiments.

### Examples 1: Measurement of Active IL-12 and IL-15 Using Polypeptide Constructs

The following polypeptide constructs listed in Table 1 were expressed in HEK cells to determine the relative activity of IL-12 and IL-15. Unless otherwise noted, any L listed in the sequence map in Table 1 can be any linker described herein.

TABLE 1
IL-12/IL-15 expression constructs

| Pep- | | |
|---|---|---|
| tide | Map | Components |
| O1 | X₁-L₁-X₂-L₂- X₂-L₃- X₂ | X₁: IL-15 |
| | | X₂: IL-12 p40 |
| | | X₃: IL-12 p35 |
| | | X₄: IL-15Rα |
| | | L₁: furin cleavage linker |
| O2 | X₁-L₁-X₂-L₂-X₃-L₃-X₄ | X₁: IL-12 p40 |
| | | X₂: IL-12 p35 |
| | | X₃: IL-15 |
| | | X₄: IL-15Rα |
| O3 | X₁-L₁-X₂-L₂-X₃-L₃-X₄ -L₄-X₅ | X₁: IL-15 |
| | | X₂: IL-15Rα first fragment |
| | | X₃: IL-12 p40 |
| | | X₄: IL-12 p35 |
| | | X₅: IL-15Rα second fragment |
| AB | X₁-L₁-X₂-L₂-X₃-L₃-X₄ | X₁: IL-15 |
| | | X₂: IL-12 p40 |
| | | X₃: IL-12 p35 |
| | | X₄: IL-15Rα |
| CD | X₁-L₁-X₂ | X₁: IL-15 |
| | | X₂: IL-15Rα |
| EF | X₁-L₁-X₂ | X₁: IL-15 |
| | | X₂: IL-15Rα |
| | | L₁: furin cleavage linker |
| H | X₁-L₁-X₂-L₂-X₃ | X₁: IL-12 p40 with IL-15 leader |
| | | X₂: IL-12 p35 |
| | | X₃: IL-15Rα second fragment |

The activity of IL-15 and IL-12 was determined by cytokine-responsive reporter cellular assays designed specifically to demonstrate IL-15 (or IL-2) and IL-12 activities. In response to native IL-15 or IL-12, engineered cell lines express enzymes that result in a colorimetric signal quantitatively measurable by a visible light detector on a plate reader. Similarly, when the constructs above were transiently expressed in these engineered cells, quantities of emitted light were indicative of IL-15 or IL-12 activities relative to soluble cytokines. The results are shown in Table 2.

TABLE 2
Relative IL-12/IL-15 activity results

| | IL-12 Activity | IL-15 Activity |
|---|---|---|
| Peptide | (relative to control) | (relative to control) |
| O1 | ++ | +++ |
| O2 | ++ | ++ |
| O3 | ++ | ++ |
| AB | ++ | ++ |
| CD | − | ++ |
| EF | − | +++ |
| H | +++ | − |

“+” means 2-10 fold activity over control
“++” means 10-100 fold activity over control
“+++” means >100 fold activity over control

### Example 2: Treatment of Cancer Using Vectors Encoding Polypeptides

A therapeutic composition comprising a vector comprising a nucleic acid molecule encoding a polypeptide as provided for herein is administered to a patient with cancer. The subject's immune system is activated and the cancer is treated. The vector can also comprise a nucleic acid molecule encoding for a tumor antigen. The vector can also comprise a nucleic acid molecule encoding for a CAR.

### Example 3: Treatment of Cancer Using Vectors Encoding Polypeptides and a Liposome

A therapeutic composition comprising a vector comprising a nucleic acid molecule encoding a polypeptide as provided for herein is administered as a formulation with a DOTAP/Cholesterol admixture to a patient with cancer. The subject's immune system is activated and the cancer is treated. The vector can also comprise a nucleic acid molecule encoding for a tumor antigen. The admixture can also comprise the pharmaceutical formulations provided for herein.

### Example 4: Treatment of Cancer Using Adoptive Cell Therapy Such as Modified Leukocytes

A T-cell comprising a polypeptide as provided for herein and a chimeric antigen receptor is administered to a subject with a cancer, such as leukemia, and the cancer is treated.

### Example 5: Treatment of Cancer Using Biologic Fusions

A therapeutic composition comprising a polypeptide as provided for herein administered to a patient with cancer. In some embodiments, the polypeptide is linked or fused to a biologically active partner, such as a protein, polypeptide, nucleic acid, lipid, carbohydrate, or any combination thereof. In some embodiments, a polypeptide is linked or fused to a targeting partner such as an antibody, an antigen, a ligand, or a ligand trap, such as a receptor domain. In some embodiments, the poly is linked or fused to an immunotherapy such as a checkpoint inhibitor. The subject's immune system is activated and the cancer is treated.

In summary, the embodiments and examples provided herein demonstrate that the polypeptides provided for herein can be used to enhance an immune response to treat a tumor or infection as provided for herein.

This specification contains numerous citations to patents, patent applications, accession numbers, and/or publications. Each is hereby incorporated by reference for all purposes.

What is claimed is:

1. A polypeptide comprising the formula of: X₁-L₁-X₂-L₂-X₃-L₃-X₄, wherein:
X₁ is a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Ra) polypeptide;
X₂ is a IL-15 polypeptide, IL-12 p40 polypeptide, IL-12 p35 polypeptide, or IL-15Ra polypeptide;
X₃ is a IL-15 polypeptide, IL-12 p40 polypeptide, IL-12 p35 polypeptide, or IL-15Ra polypeptide;
X₄ is a IL-15 polypeptide, IL-12 p40 polypeptide, IL-12 p35 polypeptide, or IL-15Ra polypeptide;
L₁, L₂, and L₃ are each, independently, a polypeptide linker that comprise the same or different polypeptide sequences,
provided each of X₁, X₂, X₃, and X₄ are different.

2. The polypeptide of claim 1, wherein:
X₁ is a IL-12 p40 polypeptide;
X₂ is a IL-12 p35 polypeptide;
X₃ is a IL-15 polypeptide; and
X₄ is a IL-15Ra polypeptide.

3. The polypeptide of claim 1, wherein:
X₁ is a IL-12 p35 polypeptide;
X₂ is a IL-12 p40 polypeptide;
X₃ is a IL-15 polypeptide; and
X₄ is a IL-15Ra polypeptide.

4. The polypeptide of claim 1, wherein:
X₁ is a IL-15 polypeptide;
X₂ is a IL-12 p40 polypeptide;
X₃ is a IL-12 p35 polypeptide; and
X₄ is a IL-15Ra polypeptide.

5. The polypeptide of claim 1, wherein:
X₁ is a IL-15 polypeptide;
X₂ is a IL-12 p35 polypeptide;
X₃ is a IL-12 p40 polypeptide; and
X₄ is a IL-15Ra polypeptide.

6. The polypeptide of claim 1, wherein:
X₁ is a IL-12 p35 polypeptide;
X₂ is a IL-15 polypeptide;
X₃ is a IL-12 p40 polypeptide; and
X₄ is a IL-15Ra polypeptide.

7. The polypeptide of claim 1, wherein:
X₁ is a IL-12 p40 polypeptide;
X₂ is a IL-15 polypeptide;
X₃ is a IL-12 p35 polypeptide; and
X₄ is a IL-15Ra polypeptide.

8. A polypeptide comprising the formula of: X₁-L₁-X₂-L₂-X₃-L₃-X₄-L₄-X₅, wherein:
X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Ra) polypeptide, a first fragment of IL-15Ra polypeptide, or a second fragment of IL-15Ra polypeptide;
X₂ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₃ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₄ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₅ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
L₁, L₂, L₃, and L₄ are each, independently, a polypeptide linker that comprise the same or different polypeptide sequences,
provided each of X₁, X₂, X₃, X₄, and X₅ are different.

9. The polypeptide of claim 8, wherein:
X₁ is a IL-15 polypeptide;
X₂ is a first fragment of IL-15Ra polypeptide;
X₃ is a IL-12 p40 polypeptide;
X₄ is a IL-12 p35 polypeptide; and
X₅ is a second fragment of IL-15Ra polypeptide.

10. The polypeptide of claim 8, wherein:
X₁ is a IL-15 polypeptide;
X₂ is a first fragment of IL-15Ra polypeptide;
X₃ is a IL-12 p35 polypeptide;
X₄ is a IL-12 p40 polypeptide; and
X₅ is a second fragment of IL-15Ra polypeptide.

11. The polypeptide of claim 8, wherein:
X₁ is a IL-15 polypeptide;
X₂ is a IL-12 p40 polypeptide;
X₃ is a first fragment of IL-15Ra polypeptide;
X₄ is a IL-12 p35 polypeptide; and
X₅ is a second fragment of IL-15Ra polypeptide.

12. The polypeptide of claim 8, wherein:
X₁ is a IL-15 polypeptide;
X₂ is a IL-12 p40 polypeptide;
X₃ is a IL-12 p35 polypeptide;
X₄ is a first fragment of IL-15Ra polypeptide; and
X₅ is a second fragment of IL-15Ra polypeptide.

13. The polypeptide of claim 8, wherein:
X₁ is a IL-15 polypeptide;
X₂ is a IL-12 p35 polypeptide;
X₃ is a IL-12 p40 polypeptide;
X₄ is a first fragment of IL-15Ra polypeptide; and
X₅ is a second fragment of IL-15Ra polypeptide.

14. The polypeptide of claim 8, wherein:
X₁ is a IL-15 polypeptide;
X₂ is a IL-12 p35 polypeptide;
X₃ is a first fragment of IL-15Ra polypeptide;
X₄ is a IL-12 p40 polypeptide; and
X₅ is a second fragment of IL-15Ra polypeptide.

15. The polypeptide of claim 8, wherein:
X₁ is a first fragment of IL-15Ra polypeptide;
X₂ is a IL-15 polypeptide;
X₃ is a IL-12 p40 polypeptide;
X₄ is a IL-12 p35 polypeptide; and
X₅ is a second fragment of IL-15Ra polypeptide.

16. The polypeptide of claim 8, wherein:
X₁ is a first fragment of IL-15Ra polypeptide;
X₂ is a IL-15 polypeptide;
X₃ is a IL-12 p35 polypeptide;
X₄ is a IL-12 p40 polypeptide; and
X₅ is a second fragment of IL-15Ra polypeptide.

17. The polypeptide of claim 8, wherein:
X₁ is a first fragment of IL-15Ra polypeptide;
X₂ is a IL-12 p40 polypeptide;
X₃ is a IL-15 polypeptide;
X₄ is a IL-12 p35 polypeptide; and
X₅ is a second fragment of IL-15Ra polypeptide.

18. The polypeptide of claim 8, wherein:
X₁ is a first fragment of IL-15Ra polypeptide;
X₂ is a IL-12 p40 polypeptide;
X₃ is a IL-12 p35 polypeptide;
X₄ is a IL-15; and
X₅ is a second fragment of IL-15Ra polypeptide.

19. The polypeptide of claim 8, wherein:
X₁ is a first fragment of IL-15Ra polypeptide;
X₂ is a IL-12 p35 polypeptide;
X₃ is a IL-12 p40 polypeptide;
X₄ is a IL-15; and
X₅ is a second fragment of IL-15Ra polypeptide.

20. The polypeptide of claim 8, wherein:
X₁ is a first fragment of IL-15Ra polypeptide;
X₂ is a IL-12 p35 polypeptide;
X₃ is a IL-15;
X₄ is a IL-12 p40 polypeptide; and
X₅ is a second fragment of IL-15Ra polypeptide.

21. The polypeptide of claim 8, wherein:
X₁ is a IL-12 p40 polypeptide;
X₂ is a IL-15 polypeptide;
X₃ is a first fragment of IL-15Ra polypeptide;
X₄ is a IL-12 p35 polypeptide; and
X₅ is a second fragment of IL-15Ra polypeptide.

22. The polypeptide of claim 8, wherein:
X₁ is a IL-12 p40 polypeptide;
X₂ is a IL-15 polypeptide;
X₃ is a IL-12 p35 polypeptide;
X₄ is a first fragment of IL-15Ra polypeptide; and
X₅ is a second fragment of IL-15Ra polypeptide.

23. The polypeptide of claim 8, wherein:
X₁ is a IL-12 p40 polypeptide;
X₂ is a first fragment of IL-15Ra polypeptide;
X₃ is a IL-15 polypeptide;
X₄ is a IL-12 p35 polypeptide; and
X₅ is a second fragment of IL-15Ra polypeptide.

24. The polypeptide of claim 8, wherein:
X₁ is a IL-12 p40 polypeptide;
X₂ is a first fragment of IL-15Ra polypeptide;
X₃ is a IL-12 p35 polypeptide;
X₄ is a IL-15; and
X₅ is a second fragment of IL-15Ra polypeptide.

25. The polypeptide of claim 8, wherein:
X₁ is a IL-12 p40 polypeptide;
X₂ is a IL-12 p35 polypeptide;
X₃ is a first fragment of IL-15Ra polypeptide;
X₄ is a IL-15; and
X₅ is a second fragment of IL-15Ra polypeptide.

26. The polypeptide of claim 8, wherein:
X₁ is a IL-12 p40 polypeptide;
X₂ is a IL-12 p35 polypeptide;
X₃ is a IL-15;
X₄ is a first fragment of IL-15Ra polypeptide; and
X₅ is a second fragment of IL-15Ra polypeptide.

27. The polypeptide of claim 8, wherein:
X₁ is a IL-12 p35 polypeptide;
X₂ is a IL-15 polypeptide;
X₃ is a first fragment of IL-15Ra polypeptide;
X₄ is a IL-12 p40 polypeptide; and
X₅ is a second fragment of IL-15Ra polypeptide.

28. The polypeptide of claim 8, wherein:
X₁ is a IL-12 p35 polypeptide;
X₂ is a IL-15 polypeptide;
X₃ is a IL-12 p40 polypeptide;
X₄ is a first fragment of IL-15Ra polypeptide; and
X₅ is a second fragment of IL-15Ra polypeptide.

29. The polypeptide of claim 8, wherein:
X₁ is a IL-12 p35 polypeptide;
X₂ is a first fragment of IL-15Ra polypeptide;
X₃ is a IL-15 polypeptide;
X₄ is a IL-12 p40 polypeptide; and
X₅ is a second fragment of IL-15Ra polypeptide.

30. The polypeptide of claim 8, wherein:
X₁ is a IL-12 p35 polypeptide;
X₂ is a first fragment of IL-15Ra polypeptide;
X₃ is a IL-12 p40 polypeptide;
X₄ is a IL-15; and
X₅ is a second fragment of IL-15Ra polypeptide.

31. The polypeptide of claim 8, wherein:
X₁ is a IL-12 p35 polypeptide;
X₂ is a IL-12 p40 polypeptide;
X₃ is a first fragment of IL-15Ra polypeptide;
X₄ is a IL-15; and
X₅ is a second fragment of IL-15Ra polypeptide.

32. The polypeptide of claim 8, wherein:
X₁ is a IL-12 p35 polypeptide;
X₂ is a IL-12 p40 polypeptide;
X₃ is a IL-15;
X₄ is a first fragment of IL-15Ra polypeptide; and
X₅ is a second fragment of IL-15Ra polypeptide.

33. A polypeptide comprising the formula of: X₁-L₁-X₂, wherein:
X₁ is a interleukin 15 (IL-15) polypeptide, interleukin 12 p40 subunit (IL-12 p40) polypeptide, interleukin 12 p35 subunit (IL-12 p35) polypeptide, or interleukin 15 receptor (IL-15Ra) polypeptide;
X₂ is a IL-15 polypeptide, IL-12 p40 polypeptide, IL-12 p35 polypeptide, or IL-15Ra polypeptide;
L₁ is a polypeptide linker,
provided X₁ and X₂ are different.

34. The polypeptide of claim 33, wherein:
X₁ is a IL-15 polypeptide; and
X₂ is a IL-15Ra polypeptide.

35. A polypeptide comprising the formula of: X₁-L₁-X₂-L₂-X₃, wherein:
X₁ is a interleukin 15 (IL-15) polypeptide, interleukin 12 p40 subunit (IL-12 p40) polypeptide, interleukin 12 p35 subunit (IL-12 p35) polypeptide, interleukin 15 receptor (IL-15Ra) polypeptide, a first fragment of IL-15Ra polypeptide, or a second fragment of IL-15Ra polypeptide;
X₂ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
X₃ is a IL-15 polypeptide, IL-12 p40 polypeptide, IL-12 p35 polypeptide, IL-15Ra polypeptide, a first fragment of IL-15Ra polypeptide, or a second fragment of IL-15Ra polypeptide;
L₁ and L₂ are each, independently, a polypeptide linker that comprise the same or different polypeptide sequences,
provided each of X₁, X₂, and X₃ are different.

36. The polypeptide of claim 35, wherein:
X₁ is a IL-12 p40 polypeptide;
X₂ is a IL-12 p35 polypeptide; and
X₃ is a second fragment of IL-15Ra polypeptide.

37. The polypeptide of claim 35, wherein:
X₁ is a IL-12 p35 polypeptide;
X₂ is a IL-12 p40 polypeptide; and
X₃ is a second fragment of IL-15Ra polypeptide.

38. The polypeptide of claim 35, wherein:
X₁ is a IL-15 polypeptide;
X₂ is a IL-12 p35 polypeptide; and
X₃ is a second fragment of IL-15Ra polypeptide.

39. The polypeptide of claim 35, wherein:
X₁ is a IL-15 polypeptide;
X₂ is a IL-12 p40 polypeptide; and
X₃ is a second fragment of IL-15Ra polypeptide.

40. The polypeptide of any one of claims 1-39, wherein the IL-12 p40 polypeptide comprises an amino acid sequence having at least 90%, 91%, 92%, 93%, 94%, 95%, 96%, 97%, 98%, or 99% identity to SEQ ID NO: 1, 2, 3, or 4, or as otherwise provided for herein.

41. The polypeptide of any one of claims 1-40, wherein the IL-12 p40 polypeptide comprises an amino acid sequence of SEQ ID NO: 1, 2, 3, or 4, or as otherwise provided for herein.

42. The polypeptide of any one of claims 1-41, wherein the IL-12 p40 polypeptide comprises a IL-15 leader sequence at the N-terminus of the polypeptide.

43. The polypeptide of claim 42, wherein the IL-15 leader sequence comprises an amino acid sequence having at least 90%, 91%, 92%, 93%, 94%, 95%, 96%, 97%, 98%, or 99% identity to SEQ ID NO: 15 or as otherwise provided for herein.

44. The polypeptide of claims 42 or 43, wherein the IL-15 leader sequence comprises an amino acid sequence of SEQ ID NO: 15 or as otherwise provided for herein.

45. The polypeptide of any one of claims 1-44, wherein the IL-12 p35 polypeptide comprises an amino acid sequence having at least 90%, 91%, 92%, 93%, 94%, 95%, 96%, 97%, 98%, or 99% identity to SEQ ID NO: 5, 6, 7, or 8, or as otherwise provided for herein.

46. The polypeptide of any one of claims 1-45, wherein the IL-12 p35 polypeptide comprises an amino acid sequence of SEQ ID NO: 5, 6, 7, or 8, or as otherwise provided for herein.

47. The polypeptide of any one of claims 1-46, wherein the IL-12 p35 polypeptide comprises a IL-15 leader sequence at the N-terminus of the polypeptide.

48. The polypeptide of claim 47, wherein the IL-15 leader sequence comprises an amino acid sequence having at least 90%, 91%, 92%, 93%, 94%, 95%, 96%, 97%, 98%, or 99% identity to SEQ ID NO: 15 or as otherwise provided for herein.

49. The polypeptide of claims 47 or 48, wherein the IL-15 leader sequence comprises an amino acid sequence of SEQ ID NO: 15 or as otherwise provided for herein.

50. The polypeptide of any one of claims 1-49, wherein the IL-15 polypeptide comprises an amino acid sequence having at least 90%, 91%, 92%, 93%, 94%, 95%, 96%, 97%, 98%, or 99% identity to SEQ ID NO: 9 or 10 or as otherwise provided for herein.

51. The polypeptide of any one of claims 1-50, wherein the IL-15 polypeptide comprises an amino acid sequence of SEQ ID NO: 9 or ‘0 or as otherwise provided for herein.

52. The polypeptide of any one of claims 1-51, wherein the IL-15Ra polypeptide comprises an amino acid sequence having at least 90%, 91%, 92%, 93%, 94%, 95%, 96%, 97%, 98%, or 99% identity to SEQ ID NO: 11 or 12 or as otherwise provided for herein.

53. The polypeptide of any one of claims 1-52, wherein the IL-15Ra polypeptide comprises an amino acid sequence of SEQ ID NO: 11 or 12 or as otherwise provided for herein.

54. The polypeptide of any one of claims 1-53 wherein the first fragment of IL-15Ra polypeptide comprises an amino acid sequence having at least 90%, 91%, 92%, 93%, 94%, 95%, 96%, 97%, 98%, or 99% identity to SEQ ID NO: 13 or as otherwise provided for herein.

55. The polypeptide of any one of claims 1-54, wherein the first fragment of IL-15Ra polypeptide comprises an amino acid sequence of SEQ ID NO: 13 or as otherwise provided for herein.

56. The polypeptide of any one of claims 1-55, wherein the second fragment of IL-15Ra polypeptide comprises an amino acid sequence having at least 90%, 91%, 92%, 93%, 94%, 95%, 96%, 97%, 98%, or 99% identity to SEQ ID NO: 14 or as otherwise provided for herein.

57. The polypeptide of any one of claims 1-52, wherein the second fragment of IL-15Ra polypeptide comprises an amino acid sequence of SEQ ID NO: 14 or as otherwise provided for herein.

58. The polypeptide of any one of claims 1-575, wherein one or more of L₁, L₂, L₃, and L₄ are each, independently, a cleavable linker.

59. The polypeptide of claim 58, wherein the cleavable linker is a furin cleavable linker, a Val-Cit linker, a Val-Gly linker, a Gly-Gly linker, an Ala-Ala-Asn linker, or a linker comprising polyethylene glycol (PEG).

60. The polypeptide of any one of claims 1-59, wherein one of L₁, L₂, L₃, and L₄ comprise a sequence of (GSGSGG)ₙ (SEQ ID NO: 16), (GGGGS)ₙ (SEQ ID NO: 17), (GGGGA)ₙ (SEQ ID NO: 18), (GGGSE)ₙ (SEQ ID NO: 19), (GGGSK)ₙ (SEQ ID NO: 20), or (AEEEK)ₙ (SEQ ID NO: 21), wherein each n is, independently, 1, 2, 3, 4, or 5, or a combination thereof.

61. The polypeptide of claim 60, wherein n is 3.

62. The polypeptide of claims 60 or 61, wherein one or more of L₁, L₂, L₃, and L₄ comprise a sequence of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 69, SEQ ID NO: 70, or a combination thereof.

63. The polypeptide of any one of claims 60-62, wherein each of L₁, L₂, L₃, and L₄ comprises a sequence of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 69, SEQ ID NO: 70, or a combination thereof.

64. The polypeptide of any one of claims 1-63, further comprising a leader peptide on the N-terminus of the polypeptide.

65. The polypeptide of claim 64, wherein the leader peptide comprises a sequence of vesicular stomatitis virus G protein (VSV-G).

66. The polypeptide of claim 64, wherein the leader peptide comprises, consists, or consists essentially of a sequence of MRISKPHLRSISIQCYLCLLLNSHFLTEA (SEQ ID NO: 15).

67. The polypeptide of claim 64, wherein the leader peptide comprises a sequence of IL-15.

68. The polypeptide of claim 64 or 67, wherein the leader peptide comprises an amino acid sequence having at least 90%, 91%, 92%, 93%, 94%, 95%, 96%, 97%, 98%, or 99% identity to SEQ ID NO: 15 or as otherwise provided for herein.

69. The polypeptide of claims 64, 67 or 68, wherein the leader peptide comprises an amino acid sequence of SEQ ID NO: 15 or as otherwise provided for herein.

70. The polypeptide of any one of claims 1-69, wherein the polypeptide comprises the sequence of SEQ ID NO: 51, a variant thereof, or as otherwise provided for herein.

71. The polypeptide of any one of claims 1-69, wherein the polypeptide comprises the sequence of SEQ ID NO: 52, a variant thereof, or as otherwise provided for herein.

72. The polypeptide of any one of claims 1-69, wherein the polypeptide comprises the sequence of SEQ ID NO: 53, a variant thereof, or as otherwise provided for herein.

73. The polypeptide of any one of claims 1-69, wherein the polypeptide comprises the sequence of SEQ ID NO: 54, a variant thereof, or as otherwise provided for herein.

74. The polypeptide of any one of claims 1-69, wherein the polypeptide comprises the sequence of SEQ ID NO: 55, a variant thereof, or as otherwise provided for herein.

75. The polypeptide of any one of claims 1-69, wherein the polypeptide comprises the sequence of SEQ ID NO: 56, a variant thereof, or as otherwise provided for herein.

76. The polypeptide of any one of claims 1-69, wherein the polypeptide comprises the sequence of SEQ ID NO: 57, a variant thereof, or as otherwise provided for herein.

77. The polypeptide of any one of claims 1-69, wherein the polypeptide comprises the sequence of SEQ ID NO: 58, a variant thereof, or as otherwise provided for herein.

78. The polypeptide of any one of claims 1-69, wherein the polypeptide comprises the sequence of SEQ ID NO: 59, a variant thereof, or as otherwise provided for herein.

79. The polypeptide of any one of claims 1-69, wherein the polypeptide comprises the sequence of SEQ ID NO: 60, a variant thereof, or as otherwise provided for herein.

80. The polypeptide of any one of claims 1-69, wherein the polypeptide comprises the sequence of SEQ ID NO: 61, a variant thereof, or as otherwise provided for herein.

81. The polypeptide of any one of claims 1-80, wherein the polypeptide is linked or fused with at least one additional molecule selected from the group consisting of a polypeptide, an antigen, a ligand, an antibody, a checkpoint inhibitor, a lipid, a carbohydrate, a nucleic acid, or any combination thereof.

82. A pharmaceutical composition comprising the polypeptide of any one of claim 1-81 and a pharmaceutical excipient.

83. The pharmaceutical composition of claim 82, wherein the pharmaceutical excipient is any pharmaceutical excipient described herein.

84. The pharmaceutical composition of claims 81 or 82, further comprising a pharmaceutical carrier.

85. The pharmaceutical composition of claim 84, wherein the pharmaceutical carrier is water.

86. A nucleic acid (e.g. DNA, RNA, cRNA, or mRNA) molecule encoding a polypeptide any one of claims 1-81.

87. The nucleic acid molecule of claim 85, wherein the nucleic acid molecule comprises of a nucleic acid sequence having at least 80%, 85%, 90%, 95%, 96%, 97%, 98%, 99% or 100% identity to SEQ ID NO: 62, SEQ ID NO: 63, SEQ ID NO: 64, SEQ ID NO: 65, SEQ ID NO: 66, or SEQ ID NO: 67.

88. A vector comprising the nucleic acid molecule of claims 86 or 87.

89. A plasmid comprising the nucleic acid molecule of claims 86 or 87.

90. A recombinant virus comprising the nucleic acid molecule of claims 86 or 87.

91. The recombinant virus of claim 90, wherein said virus is a recombinant adenovirus or lentivirus.

92. The recombinant virus of claim 91, wherein said recombinant adenovirus is replication-incompetent or replication-competent.

93. A cell comprising the polypeptide of any one of claims 1-81.

94. A cell comprising a genomic nucleic acid molecule comprising the nucleic acid molecule of claims 86 or 87.

95. The cell of claims 93 or 94, wherein the cell further comprises a chimeric antigen receptor (CAR).

96. The cell of any one of claims 93-95, wherein the cell is an immune cell.

97. The cell of claim 96, wherein the immune cell is a T-cell, a NK cell, or a dendritic cell.

98. A method for producing the cell of any one of claims 93-97, the method comprising contacting the cell with the pharmaceutical composition of any one of claims 82-85, the vector of claim 88, the plasmid of claim 89, or a virus of any one of claims 90-92.

99. The method of claim 98, wherein the contacting comprises transducing or transfecting the cell with the vector, the plasmid, or the virus.

100. The method of claim 98 or 99, wherein the vector, the plasmid, or the virus further comprise a nucleic acid molecule encoding for a chimeric antigen receptor or a tumor antigen.

101. A method of producing a cell comprising a polypeptide of any one of claims 1-81 in vivo, the method comprising administering to a subject the vector of claim 88, the plasmid of claim 89, or a virus of any one of claims 90-92, wherein the vector, plasmid, or virus transduces or transfects a cell in vivo to produce the cell comprising the polypeptide of any one of claims 1-81.

102. The method of claim 101, wherein the cell is an immune cell.

103. The method of claim 102, wherein the immune cell is, but not limited to a T-cell, a NK cell, a dendritic cell, and the like.

104. The method of any one of claims 101-103, wherein the vector, plasmid, or virus further comprise a nucleic acid molecule encoding for a chimeric antigen receptor or a tumor antigen.

105. A method for modifying an immune response in a patient, the method comprising administering to the patient the pharmaceutical composition of any one of claims 82-85, the vector of claim 88, the plasmid of claim 89, or a virus of any one of claims 90-92.

106. The method of any one of claim 105, wherein the immune response is activated.

107. The method of claim 105 or 106, wherein the vector, the plasmid, or the virus further comprise a nucleic acid molecule encoding for a chimeric antigen receptor and/or a tumor antigen.

108. A method of treating a cancer in a subject, the method comprising administering to the subject the pharmaceutical composition of any one of claims 82-85, the vector of claim 88, the plasmid of claim 89, or a virus of any one of claims 90-92.

109. The method of claim 108, wherein the cancer is as provided for herein.

110. The method of claim 108, wherein the vector, the plasmid, or the virus further comprise a nucleic acid molecule encoding for a chimeric antigen receptor and/or a tumor antigen.

111. A method of treating a disease or disorder, such as a viral infection, bacterial infection or a fungal infection, such as those provided herein, the method comprising administering to the subject the pharmaceutical composition of any one of claims 82-85, the vector of claim 88, the plasmid of claim 89, or a virus of any one of claims 90-92.


SEQUENCE LISTING (68 sequences)

SEQ ID NO: 1 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 4 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 5 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - protein, 215 aa, Mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 8 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 9 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 10 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 239 aa, Homo sapiens
LQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 12 - protein, 237 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 13 - protein, 73 aa, Homo sapiens
ITCPPPMSVEHADIWVKSYSLYSRERYICNSGFKRKAGTSSLTECVLNKATNVAHWTTPSLKCIRDPALVHQR

SEQ ID NO: 14 - protein, 164 aa, Homo sapiens
PAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 15 - protein, 29 aa, Homo sapiens
MRISKPHLRSISIQCYLCLLLNSHFLTEA

SEQ ID NO: 16 - protein, 6 aa, synthetic construct
GSGSGG

SEQ ID NO: 17 - protein, 5 aa, synthetic construct
GGGGS

SEQ ID NO: 18 - protein, 5 aa, synthetic construct
GGGGA

SEQ ID NO: 19 - protein, 5 aa, synthetic construct
GGGGE

SEQ ID NO: 20 - protein, 5 aa, synthetic construct
GGGSK

SEQ ID NO: 21 - protein, 5 aa, synthetic construct
AEEEK

SEQ ID NO: 22 - protein, 18 aa, synthetic construct
GSGSGGGSGSGGGSGSGG

SEQ ID NO: 23 - protein, 20 aa, synthetic construct
GGGGSGGGGSGGGGSGGGGS

SEQ ID NO: 24 - protein, 15 aa, synthetic construct
GGGGSGGGGSGGGGS

SEQ ID NO: 25 - protein, 5 aa, synthetic construct
GGGGS

SEQ ID NO: 26 - protein, 15 aa, synthetic construct
GGGGSGGGGSGGGGS

SEQ ID NO: 27 - protein, 5 aa, synthetic construct
GGGGS

SEQ ID NO: 28 - protein, 8 aa, synthetic construct
GGGGGGGG

SEQ ID NO: 29 - protein, 6 aa, synthetic construct
GGGGGG

SEQ ID NO: 30 - protein, 15 aa, synthetic construct
EAAAKEAAAKEAAAK

SEQ ID NO: 31 - protein, 4 aa, synthetic construct
EAAK

SEQ ID NO: 32 - protein, 46 aa, synthetic construct
AEAAAKEAAAKEAAAKEAAAKALEAEAAAKEAAAKEAAAKEAAAKA

SEQ ID NO: 33 - protein, 12 aa, synthetic construct
AEAAAKEAAAKA

SEQ ID NO: 34 - protein, 5 aa, synthetic construct
PAPAP

SEQ ID NO: 35 - skipped

SEQ ID NO: 36 - protein, 17 aa, synthetic construct
VSQTSKLTRAETVFPDV

SEQ ID NO: 37 - protein, 6 aa, synthetic construct
PLGLWA

SEQ ID NO: 38 - protein, 6 aa, synthetic construct
RVLAEA

SEQ ID NO: 39 - protein, 10 aa, synthetic construct
EDVVCCSMSY

SEQ ID NO: 40 - protein, 8 aa, synthetic construct
GGIEGRGS

SEQ ID NO: 41 - protein, 10 aa, synthetic construct
TRHRQPRGWE

SEQ ID NO: 42 - protein, 10 aa, synthetic construct
AGNRVRRSVG

SEQ ID NO: 43 - protein, 9 aa, synthetic construct
RRRRRRRRR

SEQ ID NO: 44 - protein, 4 aa, synthetic construct
GFLG

SEQ ID NO: 45 - protein, 25 aa, synthetic construct
SGGGSGGGGSGGGGSGGGSGGGSLQ

SEQ ID NO: 46 - protein, 25 aa, synthetic construct
SGGGPGGGGRAKRGGGGPSGGGSLQ

SEQ ID NO: 47 - protein, 25 aa, synthetic construct
SGGGPGGGGRARRGGGGPSGGGSLQ

SEQ ID NO: 48 - protein, 25 aa, synthetic construct
SGGGPGGGGSAGSGGGGPSGGGSLQ

SEQ ID NO: 49 - protein, 20 aa, synthetic construct
GSGGSGSGGSGSGGSGSGGS

SEQ ID NO: 50 - protein, 5 aa, synthetic construct
SGGGG

SEQ ID NO: 51 - protein, 960 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGPGGGGRAKRGGGGPSGGGSLQIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGSGGGSGSGGGSGSGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGSGSGGSGSGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 52 - protein, 963 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGPGGGGRAKRGGGGPSGGGSLQMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGSGGGSGSGGGSGSGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGSGSGGSGSGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 53 - protein, 951 aa, synthetic construct
MKCLLYLAFLFIGVNCIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGSGGGSGSGGGSGSGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGSGSGGSGSGGSGSGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 54 - protein, 955 aa, synthetic construct
MKCLLYLAFLFIGVNCMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGSGGGSGSGGGSGSGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGSGSGGSGSGGSGSGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 55 - protein, 935 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGSGGGSGSGGGSGSGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGGSGSGGSGSGGSGSGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 56 - protein, 973 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGSGGGSLQITCPPPMSVEHADIWVKSYSLYSRERYICNSGFKRKAGTSSLTECVLNKATNVAHWTTPSLKCIRDPALVHQRGSGGSGSGGSGSGGSGSGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGSGGGSGSGGGSGSGGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGGGPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 57 - protein, 970 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGSGGGSLQITCPPPMSVEHADIWVKSYSLYSRERYICNSGFKRKAGTSSLTECVLNKATNVAHWTTPSLKCIRDPALVHQRGSGGSGSGGSGSGGSGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGSGGGSGSGGGSGSGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGGPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 58 - protein, 963 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGPGGGGSAGSGGGGPSGGGSLQMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGSGGGSGSGGGSGSGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGGSGSGGSGSGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 59 - protein, 424 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 60 - protein, 424 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGPGGGGEADEGGGGPSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 61 - protein, 722 aa, synthetic construct
MRISKPHLRSISIQCYLCLLLNSHFLTEAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSGSGGGSGSGGGSGSGGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGGGPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 62 - DNA, 2892 bp, synthetic construct
  source 1..2892: mol_type = other DNA
atgaggatatctaaacctcaccttcggagtattagtatccaatgctatctctgccttctcctcaattcacatttcctcacagaagctggcatacacgtctttattcttggatgtttttctgcggggttgcctaagacagaagcaaattgggtaaacgtcatcagtgatttgaaaaagatcgaggatctcattcagagcatgcacattgacgctacgttgtatactgaatccgacgttcacccgtcatgtaaagtcactgccatgaaatgtttcctcttggagctccaggtcatctccctggaaagcggcgacgctagtattcatgatacagttgaaaacctgatcattcttgcaaacaatagcctcagtagtaacggcaacgtgaccgagtccggctgtaaggagtgtgaagaactcgaggagaagaacatcaaagaattcttgcagagtttcgttcatattgtgcaaatgttcatcaacacttcctcaggaggcgggccaggtggtgggggtagggctaagaggggtggtgggggcccaagtgggggcggctccttgcagatgtgggaattggagaaagacgtttatgttgtggaggtggactggactccagacgcgcccggtgagacagtaaacctgacttgcgacacccctgaggaggacgacattacatggacatcagaccagaggcacggggttataggatccggtaagaccctcactattaccgtgaaagagttccttgatgcaggtcagtacacttgtcataagggaggggaaaccctttcccacagtcaccttctgctgcacaagaaagagaatggtatatggtccactgaaatccttaaaaattttaagaataaaacgtttctcaaatgtgaggccccaaactatagcgggaggttcacgtgctcttggttggtccaaagaaatatggatttgaaattcaacattaaatcatctagtagctcaccagacagtagggctgtgacttgcggcatggcatcactgtccgctgaaaaagtgacccttgatcaacgagactacgaaaaatattctgtcagctgccaggaggatgtgacctgcccgactgctgaagagacgctgcccatcgaactggcgctggaggcgaggcaacagaataaatatgaaaattacagtacttcattctttatacgggacattatcaagccagaccctcccaagaacctccaaatgaaacctctcaaaaacagtcaggtagaggtctcatgggaatacccggatagttggtctacaccccattcttatttctcccttaaattttttgttaggatacaacggaagaaggagaagatgaaagaaacggaagaaggttgtaaccagaagggcgcatttctcgtagaaaaaacttcaaccgaggtacaatgtaaaggagggaacgtttgtgtccaagctcaagataggtattataacagctcatgtagcaagtgggcgtgcgtcccgtgtagggttcgcagtggatcaggtagtggcggcggatccgggagtggcgggggcagcgggtctggttcccgggtcattccagtttcagggccggccaggtgtctttcccaaagtcggaaccttctcaagacaacggatgacatggtcaagactgcgagggaaaagcttaaacactactcttgtaccgccgaggacatcgaccatgaagatattacgcgggatcaaaccagtacgttgaaaacctgtctccctcttgagcttcataagaatgagtcttgtctggcaacgagggaaacatcatcaactacacgaggtagttgcttgccgcctcagaagacttcactcatgatgacgctctgtctcgggtctatatacgaagacttgaaaatgtatcagactgaatttcaggctattaacgccgccctgcaaaatcataaccaccaacaaattatcctcgacaagggaatgctcgttgcaatcgatgaattgatgcaaagtttgaatcataacggggagacactccgacagaaaccaccggtaggtgaggcggacccgtaccgagtgaaaatgaaactttgtatcttgctccacgcgttcagcactcgggtggtcacgataaatcgagtcatgggatatttgtcatctgccgggtccggtggcagtggaagtggaggcagtggatccggtggaagtataacctgtccccctcctatgtcagtggaacacgcggatatctgggtgaagagttactcactgtactccagagagcggtatatctgtaactcaggctttaagcggaaagcgggaacctcaagcttgactgagtgtgttctcaacaaagcaaccaacgtcgcacattggactactccgagtttgaagtgtatacgagatccggcattggttcaccaacgcccggcaccacctagtaccgtaaccactgccggagtcaccccgcagccagaaagtctgtctcccagcgggaaagaaccagcggcatcatctccttcctccaataataccgcggcaacgactgccgcaatcgtgcctggatcacagttgatgccctctaagtccccatcaacggggacaacagagatcagctctcatgagagctcacacggcactccgagccaaactactgccaagaattgggaacttaccgcgtccgcgagccaccagccgcccggagtgtaccctcaaggccattctgacaccaccgtagcaattagcacatcaactgtgttgctctgcggtttgtccgccgtttcacttcttgcttgttaccttaagagtcgccagactcctcccctggcttccgttgaaatggaggctatggaggcccttcccgttacctggggtacctctagtagagatgaggacctcgagaattgctcccatcacctgtaa

SEQ ID NO: 63 - DNA, 2856 bp, synthetic construct
  source 1..2856: mol_type = other DNA
atgaaatgcctgctgtatctggcgtttctgtttattggcgtgaactgcatttgggaactgaaaaaagatgtgtatgtggtggaactggattggtatccggatgcgccgggcgaaatggtggtgctgacctgcgataccccggaagaagatggcattacctggaccctggatcagagcagcgaagtgctgggcagcggcaaaaccctgaccattcaggtgaaagaatttggcgatgcgggccagtatacctgccataaaggcggcgaagtgctgagccatagcctgctgctgctgcataaaaaagaagatggcatttggagcaccgatattctgaaagatcagaaagaaccgaaaaacaaaacctttctgcgctgcgaagcgaaaaactatagcggccgctttacctgctggtggctgaccaccattagcaccgatctgacctttagcgtgaaaagcagccgcggcagcagcgatccgcagggcgtgacctgcggcgcggcgaccctgagcgcggaacgcgtgcgcggcgataacaaagaatatgaatatagcgtggaatgccaggaagatagcgcgtgcccggcggcggaagaaagcctgccgattgaagtgatggtggatgcggtgcataaactgaaatatgaaaactataccagcagcttttttattcgcgatattattaaaccggatccgccgaaaaacctgcagctgaaaccgctgaaaaacagccgccaggtggaagtgagctgggaatatccggatacctggagcaccccgcatagctattttagcctgaccttttgcgtgcaggtgcagggcaaaagcaaacgcgaaaaaaaagatcgcgtgtttaccgataaaaccagcgcgaccgtgatttgccgcaaaaacgcgagcattagcgtgcgcgcgcaggatcgctattatagcagcagctggagcgaatgggcgagcgtgccgtgcagcggcagcggcagcggcggcggcagcggcagcggcggcggcagcggcagcggccgcaacctgccggtggcgaccccggatccgggcatgtttccgtgcctgcatcatagccagaacctgctgcgcgcggtgagcaacatgctgcagaaagcgcgccagaccctggaattttatccgtgcaccagcgaagaaattgatcatgaagatattaccaaagataaaaccagcaccgtggaagcgtgcctgccgctggaactgaccaaaaacgaaagctgcctgaacagccgcgaaaccagctttattaccaacggcagctgcctggcgagccgcaaaaccagctttatgatggcgctgtgcctgagcagcatttatgaagatctgaaaatgtatcaggtggaatttaaaaccatgaacgcgaaactgctgatggatccgaaacgccagatttttctggatcagaacatgctggcggtgattgatgaactgatgcaggcgctgaactttaacagcgaaaccgtgccgcagaaaagcagcctggaagaaccggatttttataaaaccaaaattaaactgtgcattctgctgcatgcgtttcgcattcgcgcggtgaccattgatcgcgtgatgagctatctgaacgcgagcggcagcggcggcagcggcagcggcggcagcggcagcggcggcagcggcagcggcggcagcggcattcatgtgtttattctgggctgctttagcgcgggcctgccgaaaaccgaagcgaactgggtgaacgtgattagcgatctgaaaaaaattgaagatctgattcagagcatgcatattgatgcgaccctgtataccgaaagcgatgtgcatccgagctgcaaagtgaccgcgatgaaatgctttctgctggaactgcaggtgattagcctggaaagcggcgatgcgagcattcatgataccgtggaaaacctgattattctggcgaacaacagcctgagcagcaacggcaacgtgaccgaaagcggctgcaaagaatgcgaagaactggaagaaaaaaacattaaagaatttctgcagagctttgtgcatattgtgcagatgtttattaacaccagcagcggcggcggcagcggcggcggcggcagcggcggcggcggcagcggcggcggcagcggcggcggcagcctgcagattacctgcccgccgccgatgagcgtggaacatgcggatatttgggtgaaaagctatagcctgtatagccgcgaacgctatatttgcaacagcggctttaaacgcaaagcgggcaccagcagcctgaccgaatgcgtgctgaacaaagcgaccaacgtggcgcattggaccaccccgagcctgaaatgcattcgcgatccggcgctggtgcatcagcgcccggcgccgccgagcaccgtgaccaccgcgggcgtgaccccgcagccggaaagcctgagcccgagcggcaaagaaccggcggcgagcagcccgagcagcaacaacaccgcggcgaccaccgcggcgattgtgccgggcagccagctgatgccgagcaaaagcccgagcaccggcaccaccgaaattagcagccatgaaagcagccatggcaccccgagccagaccaccgcgaaaaactgggaactgaccgcgagcgcgagccatcagccgccgggcgtgtatccgcagggccatagcgataccaccgtggcgattagcaccagcaccgtgctgctgtgcggcctgagcgcggtgagcctgctggcgtgctatctgaaaagccgccagaccccgccgctggcgagcgtggaaatggaagcgatggaagcgctgccggtgacctggggcaccagcagccgcgatgaagatctggaaaactgcagccatcatctgtaa

SEQ ID NO: 64 - DNA, 2922 bp, synthetic construct
  source 1..2922: mol_type = other DNA
atgagaatctctaaaccacacttgcgctccatcagtatacaatgctatctgtgtctgctccttaactcacacttccttaccgaagcgggtatccacgttttcatactcgggtgcttctccgctgggttgcccaaaacagaggctaactgggtgaatgttatcagcgacctcaaaaagatcgaggacttgatacagagtatgcatatagatgccaccctttataccgagagtgatgttcatccgagttgtaaggtaactgctatgaagtgttttctgcttgaacttcaggtaataagccttgaatccggagacgcaagcattcatgatacggtggaaaacctgattattctcgctaacaactccctttcaagtaatggcaacgtaaccgaatctggctgtaaagagtgcgaggagcttgaagagaaaaacatcaaagagtttttgcaatcctttgttcatattgttcaaatgtttataaacacgtcatcaggcggaggcagcggaggcggtggcagtggaggaggaggatctggaggcggatccggaggagggagtcttcagatcacgtgcccaccgccaatgagtgtcgagcacgcggatatctgggttaaatcctatagcctctactcccgggagaggtacatatgcaacagcggctttaaacgaaaagcgggaacctcctcactcacggagtgcgtcttgaacaaagcgactaacgtggcacactggactactccttctctcaaatgcatccgagaccctgcgctggtacatcagagaggaagtgggggcagtggaagcggaggaagtggcagcggcggatctggctctggtgggagtatgtgggaattggagaaggacgtatatgtggtggaggtagactggaccccagatgctccgggagagaccgtcaatcttacatgcgacactccagaggaagatgacataacttggacatctgaccaacgccatggtgtgattggatccggaaaaaccctcacgattacggtcaaggaatttctggatgcaggccagtatacttgccacaagggaggagaaacgttgtcacattctcatcttttgcttcacaagaaagaaaatggtatctggtctacagaaatcctcaagaactttaagaataaaaccttcctgaaatgcgaagctcccaattacagtgggcgatttacatgttcttggcttgtacaaaggaacatggacttgaagtttaatattaagagcagtagctcatcaccggactcacgagctgtaacatgtggaatggcgtcactgtccgcagagaaggtaacgcttgaccagcgagattacgaaaaatacagcgttagttgtcaagaagatgtcacttgtcctacggcggaggagacacttccaattgagcttgcattggaagctcgacagcagaacaaatatgaaaactacagtacgagtttctttattagggacataattaagccggatccccctaagaatttgcagatgaaacctctgaaaaacagccaggtcgaagttagctgggaatatcctgactcttggtcaaccccccactcatacttctctctgaagttctttgtgaggattcagcggaagaaagaaaaaatgaaggaaactgaggaagggtgcaatcaaaaaggtgcctttctggtggagaaaacgtctacggaggtacagtgtaagggtggaaatgtctgtgttcaagcgcaagatagatattacaactcatcctgctctaagtgggcctgcgtcccctgccgggtaaggagtggttctgggtctggcggtggatctgggagtggcggcggcagtggcagtggagggcgggtaattccggtgagtggtcccgctcgatgtctgtcccaatccagaaacttgctcaagaccactgacgatatggttaagacggcccgggagaaactgaagcactactcctgtacagcagaagacattgaccatgaagatatcacgagagatcaaacatccactcttaaaacgtgtttgccgctggagcttcataaaaacgagtcttgtttggctacacgcgagacctccagtactacgcgaggtagttgtcttccaccacaaaagactagtctcatgatgacgttgtgtttgggttctatctacgaagacctcaagatgtatcaaactgaatttcaggcaattaatgccgccctgcaaaatcataatcatcaacagatcattcttgataaggggatgctcgttgcaattgatgaactcatgcaatcacttaatcacaacggtgagacgttgcgacagaaaccaccagttggtgaggccgacccgtatcgagtcaaaatgaaactttgtatactcttgcatgcattcagtacgcgagtagtgacaattaatagggtcatgggctatcttagcagcgcatccggcggaggcggaccagctccgcccagtacagtaactacggccggggttacgccccaaccagagagtctcagtccaagtggtaaagaacctgcggcgagtagcccgagctccaacaacaccgccgcgactacagcagcgattgttccggggagccagttgatgcccagcaagtctcctagtactggcacaactgaaatatcaagtcacgaaagctctcatgggactccttcccagacaacggctaagaactgggagttgacagcaagtgcgtcacatcaaccaccaggcgtctacccacaaggccattctgacactactgtcgccattagtacgtccaccgttttgttgtgtggcctcagtgctgtgagtttgttggcctgttacctgaaaagtagacagactccaccattggcgtctgttgagatggaggccatggaggcgctcccggtgacctggggtacgtccagccgcgatgaagaccttgagaattgtagtcatcatttgtaa

SEQ ID NO: 65 - DNA, 2892 bp, synthetic construct
  source 1..2892: mol_type = other DNA
atgaggatatctaaacctcaccttcggagtattagtatccaatgctatctctgccttctcctcaattcacatttcctcacagaagctggcatacacgtctttattcttggatgtttttctgcggggttgcctaagacagaagcaaattgggtaaacgtcatcagtgatttgaaaaagatcgaggatctcattcagagcatgcacattgacgctacgttgtatactgaatccgacgttcacccgtcatgtaaagtcactgccatgaaatgtttcctcttggagctccaggtcatctccctggaaagcggcgacgctagtattcatgatacagttgaaaacctgatcattcttgcaaacaatagcctcagtagtaacggcaacgtgaccgagtccggctgtaaggagtgtgaagaactcgaggagaagaacatcaaagaattcttgcagagtttcgttcatattgtgcaaatgttcatcaacacttcctcaggaggcgggccaggtggtgggggtagtgctggttccggtggtgggggcccaagtgggggcggctccttgcagatgtgggaattggagaaagacgtttatgttgtggaggtggactggactccagacgcgcccggtgagacagtaaacctgacttgcgacacccctgaggaggacgacattacatggacatcagaccagaggcacggggttataggatccggtaagaccctcactattaccgtgaaagagttccttgatgcaggtcagtacacttgtcataagggaggggaaaccctttcccacagtcaccttctgctgcacaagaaagagaatggtatatggtccactgaaatccttaaaaattttaagaataaaacgtttctcaaatgtgaggccccaaactatagcgggaggttcacgtgctcttggttggtccaaagaaatatggatttgaaattcaacattaaatcatctagtagctcaccagacagtagggctgtgacttgcggcatggcatcactgtccgctgaaaaagtgacccttgatcaacgagactacgaaaaatattctgtcagctgccaggaggatgtgacctgcccgactgctgaagagacgctgcccatcgaactggcgctggaggcgaggcaacagaataaatatgaaaattacagtacttcattctttatacgggacattatcaagccagaccctcccaagaacctccaaatgaaacctctcaaaaacagtcaggtagaggtctcatgggaatacccggatagttggtctacaccccattcttatttctcccttaaattttttgttaggatacaacggaagaaggagaagatgaaagaaacggaagaaggttgtaaccagaagggcgcatttctcgtagaaaaaacttcaaccgaggtacaatgtaaaggagggaacgtttgtgtccaagctcaagataggtattataacagctcatgtagcaagtgggcgtgcgtcccgtgtagggttcgcagtggatcaggtagtggcggcggatccgggagtggcgggggcagcgggtctggttcccgggtcattccagtttcagggccggccaggtgtctttcccaaagtcggaaccttctcaagacaacggatgacatggtcaagactgcgagggaaaagcttaaacactactcttgtaccgccgaggacatcgaccatgaagatattacgcgggatcaaaccagtacgttgaaaacctgtctccctcttgagcttcataagaatgagtcttgtctggcaacgagggaaacatcatcaactacacgaggtagttgcttgccgcctcagaagacttcactcatgatgacgctctgtctcgggtctatatacgaagacttgaaaatgtatcagactgaatttcaggctattaacgccgccctgcaaaatcataaccaccaacaaattatcctcgacaagggaatgctcgttgcaatcgatgaattgatgcaaagtttgaatcataacggggagacactccgacagaaaccaccggtaggtgaggcggacccgtaccgagtgaaaatgaaactttgtatcttgctccacgcgttcagcactcgggtggtcacgataaatcgagtcatgggatatttgtcatctgccgggtccggtggcagtggaagtggaggcagtggatccggtggaagtataacctgtccccctcctatgtcagtggaacacgcggatatctgggtgaagagttactcactgtactccagagagcggtatatctgtaactcaggctttaagcggaaagcgggaacctcaagcttgactgagtgtgttctcaacaaagcaaccaacgtcgcacattggactactccgagtttgaagtgtatacgagatccggcattggttcaccaacgcccggcaccacctagtaccgtaaccactgccggagtcaccccgcagccagaaagtctgtctcccagcgggaaagaaccagcggcatcatctccttcctccaataataccgcggcaacgactgccgcaatcgtgcctggatcacagttgatgccctctaagtccccatcaacggggacaacagagatcagctctcatgagagctcacacggcactccgagccaaactactgccaagaattgggaacttaccgcgtccgcgagccaccagccgcccggagtgtaccctcaaggccattctgacaccaccgtagcaattagcacatcaactgtgttgctctgcggtttgtccgccgtttcacttcttgcttgttaccttaagagtcgccagactcctcccctggcttccgttgaaatggaggctatggaggcccttcccgttacctggggtacctctagtagagatgaggacctcgagaattgctcccatcacctgtaa

SEQ ID NO: 66 - DNA, 1275 bp, synthetic construct
  source 1..1275: mol_type = other DNA
atgagaatctctaaaccacacttgcgctccatcagtatacaatgctatctgtgtctgctccttaactcacacttccttaccgaagcgggtatccacgttttcatactcgggtgcttctccgctgggttgcccaaaacagaggctaactgggtgaatgttatcagcgacctcaaaaagatcgaggacttgatacagagtatgcatatagatgccaccctttataccgagagtgatgttcatccgagttgtaaggtaactgctatgaagtgttttctgcttgaacttcaggtaataagccttgaatccggagacgcaagcattcatgatacggtggaaaacctgattattctcgctaacaactccctttcaagtaatggcaacgtaaccgaatctggctgtaaagagtgcgaggagcttgaagagaaaaacatcaaagagtttttgcaatcctttgttcatattgttcaaatgtttataaacacgtcatcaggcggaggcagcggaggcggtggcagtggaggaggaggatctggaggcggatccggaggagggagtcttcagatcacgtgcccaccgccaatgagtgtcgagcacgcggatatctgggttaaatcctatagcctctactcccgggagaggtacatatgcaacagcggctttaaacgaaaagcgggaacctcctcactcacggagtgcgtcttgaacaaagcgactaacgtggcacactggactactccttctctcaaatgcatccgagaccctgcgctggtacatcagagaccagctccgcccagtacagtaactacggccggggttacgccccaaccagagagtctcagtccaagtggtaaagaacctgcggcgagtagcccgagctccaacaacaccgccgcgactacagcagcgattgttccggggagccagttgatgcccagcaagtctcctagtactggcacaactgaaatatcaagtcacgaaagctctcatgggactccttcccagacaacggctaagaactgggagttgacagcaagtgcgtcacatcaaccaccaggcgtctacccacaaggccattctgacactactgtcgccattagtacgtccaccgttttgttgtgtggcctcagtgctgtgagtttgttggcctgttacctgaaaagtagacagactccaccattggcgtctgttgagatggaggccatggaggcgctcccggtgacctggggtacgtccagccgcgatgaagaccttgagaattgtagtcatcatttgtaa

SEQ ID NO: 67 - DNA, 1275 bp, synthetic construct
  source 1..1275: mol_type = other DNA
atgaggatatctaaacctcaccttcggagtattagtatccaatgctatctctgccttctcctcaattcacatttcctcacagaagctggcatacacgtctttattcttggatgtttttctgcggggttgcctaagacagaagcaaattgggtaaacgtcatcagtgatttgaaaaagatcgaggatctcattcagagcatgcacattgacgctacgttgtatactgaatccgacgttcacccgtcatgtaaagtcactgccatgaaatgtttcctcttggagctccaggtcatctccctggaaagcggcgacgctagtattcatgatacagttgaaaacctgatcattcttgcaaacaatagcctcagtagtaacggcaacgtgaccgagtccggctgtaaggagtgtgaagaactcgaggagaagaacatcaaagaattcttgcagagtttcgttcatattgtgcaaatgttcatcaacacttcctcaggaggcgggccaggtggtgggggtgaagctgacgagggtggtgggggcccaagtgggggcggctccttgcagataacctgtccccctcctatgtcagtggaacacgcggatatctgggtgaagagttactcactgtactccagagagcggtatatctgtaactcaggctttaagcggaaagcgggaacctcaagcttgactgagtgtgttctcaacaaagcaaccaacgtcgcacattggactactccgagtttgaagtgtatacgagatccggcattggttcaccaacgcccggcaccacctagtaccgtaaccactgccggagtcaccccgcagccagaaagtctgtctcccagcgggaaagaaccagcggcatcatctccttcctccaataataccgcggcaacgactgccgcaatcgtgcctggatcacagttgatgccctctaagtccccatcaacggggacaacagagatcagctctcatgagagctcacacggcactccgagccaaactactgccaagaattgggaacttaccgcgtccgcgagccaccagccgcccggagtgtaccctcaaggccattctgacaccaccgtagcaattagcacatcaactgtgttgctctgcggtttgtccgccgtttcacttcttgcttgttaccttaagagtcgccagactcctcccctggcttccgttgaaatggaggctatggaggcccttcccgttacctggggtacctctagtagagatgaggacctcgagaattgctcccatcacctgtaa

SEQ ID NO: 68 - DNA, 2169 bp, synthetic construct
  source 1..2169: mol_type = other DNA
atgagaatctctaaaccacacttgcgctccatcagtatacaatgctatctgtgtctgctccttaactcacacttccttaccgaagcgatgtgggaattggagaaggacgtatatgtggtggaggtagactggaccccagatgctccgggagagaccgtcaatcttacatgcgacactccagaggaagatgacataacttggacatctgaccaacgccatggtgtgattggatccggaaaaaccctcacgattacggtcaaggaatttctggatgcaggccagtatacttgccacaagggaggagaaacgttgtcacattctcatcttttgcttcacaagaaagaaaatggtatctggtctacagaaatcctcaagaactttaagaataaaaccttcctgaaatgcgaagctcccaattacagtgggcgatttacatgttcttggcttgtacaaaggaacatggacttgaagtttaatattaagagcagtagctcatcaccggactcacgagctgtaacatgtggaatggcgtcactgtccgcagagaaggtaacgcttgaccagcgagattacgaaaaatacagcgttagttgtcaagaagatgtcacttgtcctacggcggaggagacacttccaattgagcttgcattggaagctcgacagcagaacaaatatgaaaactacagtacgagtttctttattagggacataattaagccggatccccctaagaatttgcagatgaaacctctgaaaaacagccaggtcgaagttagctgggaatatcctgactcttggtcaaccccccactcatacttctctctgaagttctttgtgaggattcagcggaagaaagaaaaaatgaaggaaactgaggaagggtgcaatcaaaaaggtgcctttctggtggagaaaacgtctacggaggtacagtgtaagggtggaaatgtctgtgttcaagcgcaagatagatattacaactcatcctgctctaagtgggcctgcgtcccctgccgggtaaggagtggttctgggtctggcggtggatctgggagtggcggcggcagtggcagtggagggcgggtaattccggtgagtggtcccgctcgatgtctgtcccaatccagaaacttgctcaagaccactgacgatatggttaagacggcccgggagaaactgaagcactactcctgtacagcagaagacattgaccatgaagatatcacgagagatcaaacatccactcttaaaacgtgtttgccgctggagcttcataaaaacgagtcttgtttggctacacgcgagacctccagtactacgcgaggtagttgtcttccaccacaaaagactagtctcatgatgacgttgtgtttgggttctatctacgaagacctcaagatgtatcaaactgaatttcaggcaattaatgccgccctgcaaaatcataatcatcaacagatcattcttgataaggggatgctcgttgcaattgatgaactcatgcaatcacttaatcacaacggtgagacgttgcgacagaaaccaccagttggtgaggccgacccgtatcgagtcaaaatgaaactttgtatactcttgcatgcattcagtacgcgagtagtgacaattaatagggtcatgggctatcttagcagcgcatccggcggaggcggaccagctccgcccagtacagtaactacggccggggttacgccccaaccagagagtctcagtccaagtggtaaagaacctgcggcgagtagcccgagctccaacaacaccgccgcgactacagcagcgattgttccggggagccagttgatgcccagcaagtctcctagtactggcacaactgaaatatcaagtcacgaaagctctcatgggactccttcccagacaacggctaagaactgggagttgacagcaagtgcgtcacatcaaccaccaggcgtctacccacaaggccattctgacactactgtcgccattagtacgtccaccgttttgttgtgtggcctcagtgctgtgagtttgttggcctgttacctgaaaagtagacagactccaccattggcgtctgttgagatggaggccatggaggcgctcccggtgacctggggtacgtccagccgcgatgaagaccttgagaattgtagtcatcatttgtaa